(19) United States
(12) Patent Application Publication
(10) Pub. No.: US 2025/0313621 A1
(43) Pub. Date: Oct. 9, 2025

(54) ANTI-PAR2 ANTIBODIES

(71) Applicant: NXERA PHARMA UK LIMITED, Cambridge (GB)

(72) Inventors: Catherine HUTCHINGS, Cambridge (GB); Greg OSBORNE, Cambridge (GB); Oliver SCHLENKER, Cambridge (GB); Krzysztof OKRASA, Cambridge (GB); Andrei ZHUKOV, Cambridge (GB); Annika SCHMID, Planegg (DE); Alexandra KRAUS, Planegg (DE); Dorethée RÜHLE, Planegg (DE)

(21) Appl. No.: 18/850,322

(22) PCT Filed: Mar. 24, 2023
(86) PCT No.: PCT/GB2023/050760

§ 371 Date: Sep. 24, 2024

(30) Foreign Application Priority Data
Mar. 24, 2022 (EP) 2204159.4

(51) Int. Cl. C07K 16/28; A61K 39/00; A61K 45/06

(52) U.S. Cl. CPC C07K 16/28; A61K 45/06; A61K 2039/505; C07K 2317/33; C07K 2317/565; C07K 2317/76; C07K 2317/92; C07K 2317/94

(57) ABSTRACT

The present disclosure provides antibodies and antigen-binding fragments capable of binding to human PAR2, including antibodies capable of acting as dual inhibitors of PAR2. Dual inhibitor antibodies are able to inhibit both protease cleavage mediated activation of PAR2 (e.g. by trypsin) and peptide mediated activation of PAR2 (e.g. by PAR2-AP or PAR1-AP). The disclosure further provides methods for making and using the antibodies and antigen-binding fragments.

### RELATED APPLICATIONS

This application is a National Stage application of International Application No. PCT/GB2023/050760, filed Mar. 24, 2023 which claims priority under 35 U.S.C. § 119 to United Kingdom Patent Application No. 2204159.4, filed Mar. 24, 2022.

### SEQUENCE LISTING

The Sequence Listing associated with this application is provided in XML format in lieu of paper copy, and is hereby incorporated by reference into the specification. The name of the XML file containing the Sequence Listing is “Sequence Listing-20240906-RMC00451WO”. The XML file is 97.7 KB, was created on Sep. 6, 2024, and was submitted electronically with the filing of the International Application.

### FIELD OF THE INVENTION

This invention relates to an antibody or antigen-binding fragment capable of binding to human PAR2. The invention further relates to antibodies, which specifically bind to an epitope of the human PAR2 receptor and block, antagonise, inhibit or prevent activation of human PAR2. The invention relates to methods for making, methods for using and pharmaceutical compositions comprising said antibodies.

### BACKGROUND OF THE INVENTION

Chronic pain and chronic inflammation are two of the biggest burdens on global health. Chronic pain alone affects approximately 50 million American adults or 20% of the population. Chronic pain is a debilitating condition which is defined as pain that persists and is experienced most days or every day for 6 months or more (https://uspainfoundation.org/wp-content/uploads/2018/09/Chronic-pain-facts-infographic.pdf). Chronic inflammation plays a central role in diseases that contribute to a high number of deaths including cancer, cardiovascular disease and diabetes. It has been predicted that chronic diseases will account for approximately three-quarters of all death worldwide by 2020 (Helamo, Delil and Dileba, 2017). Despite chronic pain being a global burden, patients only receive a 30% pain reduction from current available treatments (Rice, Smith and Blyth, 2016).

Protease Activated Receptor 2 (PAR2) is a G protein-coupled receptor that belongs to a family of Protease-Activated Receptors (PAR). PAR2 is ascribed a critical role in inflammation, pain and other pathophysiological responses, where elevated levels of proteases are found. PAR2 is widely expressed with especially high levels in pancreas, liver, kidney, small intestine and colon. Moderate expression is detected in numerous epithelial and endothelial cells and organs, with limited evidence for expression in brain or skeletal muscle. In addition, PAR2 is also expressed on immune and inflammatory cells, such as T-cells, monocytes, macrophages, neutrophils, mast cells, and eosinophils.

The literature suggests that blockade of PAR2 is likely to create clinical benefit in atopic dermatitis, asthma, cancer (various including breast, melanoma, head and neck), pain (inflammatory, post-operative, neuropathic, fracture, gout, cancer, gastrointestinal associated with inflammatory bowel disease), rheumatoid arthritis and associated uveitis, scleroderma, systemic lupus erythematosus, osteoarthritis, polymyalgia rheumatica, ankylosing spondylitis, Reiter's disease, psoriatic arthritis, chronic Lyme arthritis, Still's disease, dermatomyositis, inclusion body myositis, polymyositis, lymphangioleiomyomatosis, allergic rhinoconjunctivitis (AR), eosinophilic esophagitis (EoE) and diseases associated with epithelial barrier function (reviewed in Yau et al., 2013; Heuberger and Schuepbach, 2019).

PAR2 antagonists are thus thought likely to provide benefit to a wide variety of patients and to have a potential to alleviate pain and/or inflammation-related conditions. Hence, PAR2 is regarded as a valuable therapeutic target for the treatment of several disease indications.

There is a need to identify a therapeutic moiety that can specifically inhibit PAR2. Such an agent would be particularly desirable if it could inhibit all mechanisms of PAR2 activation.

### SUMMARY OF THE INVENTION

Provided herein are antibodies and antigen-binding fragments thereof that bind PAR2. The antibodies and antigen-binding fragments of the disclosure are useful, inter alia, for inhibiting PAR2-mediated signalling and for treating diseases and disorders caused by or related to PAR2 activity and/or signalling.

The antibodies provided herein, or antigen-binding fragments thereof, specifically bind to and inhibit the activity of PAR2, wherein the antibody or fragment thereof binds to an epitope comprising an extracellular loop (ECL) and an N-terminal segment of PAR2 including helices 0 and 1. It is believed that binding to both of these regions can lead to comprehensive functional inhibition of PAR2 activity. Thus the antibodies provided herein are dual-active in that they are able to inhibit both protease cleavage mediated activation of PAR2 (e.g. by trypsin) and peptide mediated activation of PAR2 (e.g. by PAR2-AP or PAR1-AP).

In embodiments, the antibody or antigen-binding fragment thereof, specifically bind to a discontinuous epitope of PAR2, wherein the epitope comprises one or more regions of non-helical Segment1 preceding Helix0/1, the Helix0/1 region and ECL3, optionally wherein the regions of Segment1, Helix0/1 and ECL3 are selected from V55-F77, L306-Y311 and F312-Y326 of PAR2 when numbered in accordance with the human PAR2 sequence of SEQ ID NO: 1.

In embodiments, the antibody or antigen-binding fragment thereof specifically binds to and inhibits the activity of PAR2, and comprises a VH domain comprising a HCDR3, wherein: (a) a HCDR3 comprising or consisting of the amino acid sequence of SEQ ID NO: 5, 22 or 30; or SEQ ID NO: 5, 22 or 30 with 3, 2 or 1 amino acid substitutions thereto; (b) a HCDR3 comprising an amino acid sequence that is at least 80%, 85%, 90%, 92%, 93%, 95%, 97%, 98%, 99% or 100% identical to SEQ ID NO: 5, 22 or 30; or d) a HCDR3 amino acid sequence is as defined by Kabat or Chothia and is from a VH domain selected from SEQ ID NO: 2, 10, 13, 16, 19 or 27.

In embodiments, the antibody or antigen-binding fragment thereof comprises a VH domain, wherein the VH domain comprises: i. a HCDR1 amino acid sequence selected from SEQ ID NO: 3, 11, 14, 17, 20 or 28, optionally with 3, 2 or 1 amino acid substitution(s) thereto; and/or ii. a HCDR2 amino acid sequence selected from SEQ ID NO: 4, 12, 15, 18, 21 or 29, optionally with 3, 2 or 1 amino acid substitution(s) thereto.

In embodiments, the antibody or antigen-binding fragment thereof comprises a VL domain, optionally a VL domain comprising an amino acid sequence that is at least 80%, 85%, 90%, 92%, 93%, 95%, 97%, 98%, 99% or 100% identical to SEQ ID NO: 6, 23 or 31.

In embodiments, the antibody or antigen-binding fragment thereof comprises a LCDR3, wherein a) the LCDR3 amino acid sequence is selected from: SEQ ID NO: 9, 26 or 33, optionally with 3, 2 or 1 amino acid substitution(s) thereto; or b) the LCDR3 amino acid sequence as defined by Chothia or Kabat and is from a VL domain according to SEQ ID NO: 6, 23 or 31, optionally wherein the LCDR3 sequence comprises 3, 2 or 1 amino acid substitution(s).

In embodiments, the antibody or antigen-binding fragment thereof comprises a VL domain, wherein the VL domain comprises: a) i) a LCDR1 amino acid sequence of SEQ ID NO: 7, 24 or 32, optionally with 3, 2 or 1 amino acid substitution(s) thereto; or ii) a LCDR1 amino acid sequence, as defined by Chothia or Kabat, from a VL domain according to SEQ ID NO: 6, 23 or 31, optionally wherein the LCDR1 sequence comprises 3, 2 or 1 amino acid substitution(s); and/or b) i) a LCDR2 amino acid sequence of SEQ ID NO: 8 or 25, optionally with 3, 2 or 1 amino acid substitution(s) thereto; or ii) a LCDR2 amino acid sequence, as defined by Chothia or Kabat, from a VL domain according to SEQ ID NO: 6, 23 or 31, optionally wherein the LCDR2 sequence comprises 3, 2 or 1 amino acid substitution(s).

Provided herein is an antibody or antigen-binding fragment thereof which specifically binds to PAR2 comprising a VH region selected from SEQ ID NO: 2, 10, 13 and 16, 19 or 27, or an amino acid sequence that is at least 80%, 85%, 90%, 92%, 93%, 95%, 97%, 98%, 99% or 100% identical thereto; and a VL region according to SEQ ID NO: 6, 23 or 31, or an amino acid sequence that is at least 80%, 85%, 90%, 92%, 93%, 95%, 97%, 98%, 99% or 100% identical thereto.

In embodiments, the antibody or antigen-binding fragment thereof comprises a VH region, wherein the VH region comprises an amino acid sequence that is at least 80%, 85%, 90%, 92%, 93%, 95%, 97%, 98%, 99% or 100% identical to SEQ ID NO: 2. In embodiments, the antibody or antigen-binding fragment thereof comprises a VH region, wherein the VH region comprises an amino acid sequence that is at least 80%, 85%, 90%, 92%, 93%, 95%, 97%, 98%, 99% or 100% identical to SEQ ID NO: 10. In embodiments, the antibody or antigen-binding fragment thereof comprises a VH region, wherein the VH region comprises an amino acid sequence that is at least 80%, 85%, 90%, 92%, 93%, 95%, 97%, 98%, 99% or 100% identical to SEQ ID NO: 13. In embodiments, the antibody or antigen-binding fragment thereof comprises a VH region, wherein the VH region comprises an amino acid sequence that is at least 80%, 85%, 90%, 92%, 93%, 95%, 97%, 98%, 99% or 100% identical to SEQ ID 16. In embodiments, the antibody or antigen-binding fragment thereof comprises a VH region, wherein the VH region comprises an amino acid sequence that is at least 80%, 85%, 90%, 92%, 93%, 95%, 97%, 98%, 99% or 100% identical to SEQ ID 19. In embodiments, the antibody or antigen-binding fragment thereof comprises a VH region, wherein the VH region comprises an amino acid sequence that is at least 80%, 85%, 90%, 92%, 93%, 95%, 97%, 98%, 99% or 100% identical to SEQ ID NO: 27. In embodiments, the antibody or antigen-binding fragment thereof comprises a V_{L} region, wherein the V_{L} region comprises an amino acid sequence that is at least 65%, 70%, 75%, 80%, 85%, 90%, 92%, 93%, 95%, 97%, 98%, 99% or 100% identical to SEQ ID NO: 23. In embodiments, the antibody or antigen-binding fragment thereof comprises a V_{L} region, wherein the V_{L} region comprises an amino acid sequence that is at least 65%, 70%, 75%, 80%, 85%, 90%, 92%, 93%, 95%, 97%, 98%, 99% or 100% identical to SEQ ID NO: 31.

In embodiments, the antibody or antigen-binding fragment thereof comprises a V_{H} region, wherein the V_{H} region comprises an amino acid sequence which is identical or at least 90% identical to SEQ ID NO: 2 and the V_{L} region comprises an amino acid sequence which is identical or at least 90% identical to SEQ ID NO: 6. In embodiments, the antibody or antigen-binding fragment thereof comprises a V_{H} region, wherein the V_{H} region comprises an amino acid sequence which is identical or at least 90% identical to SEQ ID NO: 10 and the V_{L} region comprises an amino acid sequence which is identical or at least 90% identical to SEQ ID NO: 6. In embodiments, the antibody or antigen-binding fragment thereof comprises a V_{H} region, wherein the V_{H} region comprises an amino acid sequence which is identical or at least 90% identical SEQ ID NO: 13 and the V_{L} region comprises an amino acid sequence which is identical or at least 90% identical to SEQ ID NO: 6. In embodiments, the antibody or antigen-binding fragment thereof comprises a V_{H} region, wherein the V_{H} region comprises an amino acid sequence which is identical or at least 90% identical to SEQ ID NO: 16 and the VL region comprises an amino acid sequence which is identical or at least 90% identical to SEQ ID NO: 6. In embodiments, the antibody or antigen-binding fragment thereof comprises a V_{H} region, wherein the V_{H} region comprises an amino acid sequence which is identical or at least 90% identical to SEQ ID NO: 19 and the V_{L} region comprises an amino acid sequence which is identical or at least 90% identical to SEQ ID NO: 23. In embodiments, the antibody or antigen-binding fragment thereof comprises a V_{H} region, wherein the V_{H} region comprises an amino acid sequence which is identical or at least 90% identical to SEQ ID NO: 27 and the V_{L} region comprises an amino acid sequence which is identical or at least 90% identical to SEQ ID NO: 31.

In embodiments, the antibody or antigen-binding fragment thereof specifically binds to PAR2 and comprises a heavy chain variable domain (V_{H}) and a light chain variable domain (VL), wherein the V_{H} comprises: (a) the HCDR1 having the amino acid sequence of SEQ ID NO: 3 or SEQ ID NO: 3 comprising 3, 2 or 1 amino acid substitution(s); (b) the HCDR2 having the amino acid sequence of SEQ ID NO: 4 or SEQ ID NO: 4 comprising 3, 2 or 1 amino acid substitution(s); (c) the HCDR3 having the amino acid sequence of SEQ ID NO: 5 or SEQ ID NO: 5 comprising 3, 2 or 1 amino acid substitution(s); and wherein the V_{L} comprises: (d) the LCDR1 having the amino acid sequence of SEQ ID NO: 7 or SEQ ID NO: 7 comprising 3, 2 or 1 amino acid substitution(s); (e) the LCDR2 having the amino acid sequence of SEQ ID NO: 8 or SEQ ID NO: 8 comprising 3, 2 or 1 amino acid substitution(s); and (f) the LCDR3 having the amino acid sequence of SEQ ID NO: 9 or SEQ ID NO: 9 comprising 3, 2 or 1 amino acid substitution(s).

In embodiments, the antibody or antigen-binding fragment thereof specifically binds to PAR2 and comprises a heavy chain variable domain (V_{H}) and a light chain variable domain (VL), wherein the V_{H} comprises: (a) the HCDR1 having the amino acid sequence of SEQ ID NO: 11 or SEQ ID NO: 11 comprising 3, 2 or 1 amino acid substitution(s); (b) the HCDR2 having the amino acid sequence of SEQ ID NO: 12 or SEQ ID NO: 12 comprising 3, 2 or 1 amino acid substitution(s); and (c) the HCDR3 having the amino acid sequence of SEQ ID NO: 5 or SEQ ID NO: 5 comprising 3, 2 or 1 amino acid substitution(s); and wherein the VL comprises: (d) the LCDR1 having the amino acid sequence of SEQ ID NO: 7 or SEQ ID NO: 7 comprising 3, 2 or 1 amino acid substitution(s), (e) the LCDR2 having the amino acid sequence of SEQ ID NO: 8 or SEQ ID NO: 8 comprising 3, 2 or 1 amino acid substitution(s), and (f) the LCDR3 having the amino acid sequence of SEQ ID NO: 9 or SEQ ID NO: 9 comprising 3, 2 or 1 amino acid substitution(s).

In embodiments, the antibody or antigen-binding fragment thereof specifically binds to PAR2 and comprises a heavy chain variable domain (V_{H}) and a light chain variable domain (VL), wherein the V_{H} comprises: (a) the HCDR1 having the amino acid sequence of SEQ ID NO: 14 or SEQ ID NO: 14 comprising 3, 2 or 1 amino acid substitution(s); (b) the HCDR2 having the amino acid sequence of SEQ ID NO: 15 or SEQ ID NO: 15 comprising 3, 2 or 1 amino acid substitution(s); (c) the HCDR3 having the amino acid sequence of SEQ ID NO: 5 or SEQ ID NO: 5 comprising 3, 2 or 1 amino acid substitution(s); and wherein the VL comprises: (d) the LCDR1 having the amino acid sequence of SEQ ID NO: 7 or SEQ ID NO: 7 comprising 3, 2 or 1 amino acid substitution(s), (e) the LCDR2 having the amino acid sequence of SEQ ID NO: 8 or SEQ ID NO: 8 comprising 3, 2 or 1 amino acid substitution(s), and (f) the LCDR3 having the amino acid sequence of SEQ ID NO: 9 or SEQ ID NO: 9 comprising 3, 2 or 1 amino acid substitution(s).

In embodiments, the antibody or antigen-binding fragment thereof specifically binds to PAR2 and comprises a heavy chain variable domain (V_{H}) and a light chain variable domain (VL), wherein the V_{H} comprises: (a) the HCDR1 having the amino acid sequence of SEQ ID NO: 17 or SEQ ID NO: 17 comprising 3, 2 or 1 amino acid substitution(s); (b) the HCDR2 having the amino acid sequence of SEQ ID NO: 18 or SEQ ID NO: 18 comprising 3, 2 or 1 amino acid substitution(s); (c) the HCDR3 having the amino acid sequence of SEQ ID NO: 5 or SEQ ID NO: 5 comprising 3, 2 or 1 amino acid substitution(s); and wherein the VL comprises: (d) the LCDR1 having the amino acid sequence of SEQ ID NO: 7 or SEQ ID NO: 7 comprising 3, 2 or 1 amino acid substitution(s), (e) the LCDR2 having the amino acid sequence of SEQ ID NO: 8 or SEQ ID NO: 8 comprising 3, 2 or 1 amino acid substitution(s), and (f) the LCDR3 having the amino acid sequence of SEQ ID NO: 9 or SEQ ID NO: 9 comprising 3, 2 or 1 amino acid substitution(s).

In embodiments, the antibody or antigen-binding fragment thereof specifically binds to PAR2 and comprises a heavy chain variable domain (V_{H}) and a light chain variable domain (VL), wherein the V_{H} comprises: (a) the HCDR1 having the amino acid sequence of SEQ ID NO: 20 or SEQ ID NO: 20 comprising 3, 2 or 1 amino acid substitution(s); (b) the HCDR2 having the amino acid sequence of SEQ ID NO: 21 or SEQ ID NO: 21 comprising 3, 2 or 1 amino acid substitution(s); (c) the HCDR3 having the amino acid sequence of SEQ ID NO: 22 or SEQ ID NO: 22 comprising 3, 2 or 1 amino acid substitution(s); and wherein the VL comprises: (d) the LCDR1 having the amino acid sequence of SEQ ID NO: 24 or SEQ ID NO: 24 comprising 3, 2 or 1 amino acid substitution(s), (e) the LCDR2 having the amino acid sequence of SEQ ID NO: 25 or SEQ ID NO: 25 comprising 3, 2 or 1 amino acid substitution(s), and (f) the LCDR3 having the amino acid sequence of SEQ ID NO: 26 or SEQ ID NO: 26 comprising 3, 2 or 1 amino acid substitution(s).

In embodiments, the antibody or antigen-binding fragment thereof specifically binds to PAR2 and comprises a heavy chain variable domain (V_{H}) and a light chain variable domain (VL), wherein the V_{H} comprises: (a) the HCDR1 having the amino acid sequence of SEQ ID NO: 28 or SEQ ID NO: 28 comprising 3, 2 or 1 amino acid substitution(s); (b) the HCDR2 having the amino acid sequence of SEQ ID NO: 29 or SEQ ID NO: 29 comprising 3, 2 or 1 amino acid substitution(s); (c) the HCDR3 having the amino acid sequence of SEQ ID NO: 30 or SEQ ID NO: 30 comprising 3, 2 or 1 amino acid substitution(s); and wherein the VL comprises: (d) the LCDR1 having the amino acid sequence of SEQ ID NO: 32 or SEQ ID NO: 32 comprising 3, 2 or 1 amino acid substitution(s), (e) the LCDR2 having the amino acid sequence of SEQ ID NO: 25 or SEQ ID NO: 25 comprising 3, 2 or 1 amino acid substitution(s), and (f) the LCDR3 having the amino acid sequence of SEQ ID NO: 33 or SEQ ID NO: 33 comprising 3, 2 or 1 amino acid substitution(s).

In embodiments, the antibody or antigen-binding fragment thereof comprises an amino acid sequence that is at least 80%, 85%, 90%, 95% or 100% identical to SEQ ID NO: 77. In embodiments, the antibody or antigen-binding fragment thereof comprises the amino acid sequence of SEQ ID NO: 77. In embodiments, the antibody or antigen-binding fragment thereof comprises an amino acid sequence that is at least 80%, 85%, 90%, 95% or 100% identical to SEQ ID NO: 78. In embodiments, the antibody or antigen-binding fragment thereof comprises the amino acid sequence of SEQ ID NO: 78. In embodiments, the antibody or antigen-binding fragment thereof comprises an amino acid sequence that is at least 80%, 85%, 90%, 95% or 100% identical to SEQ ID NO: 79. In embodiments, the antibody or antigen-binding fragment thereof comprises the amino acid sequence of SEQ ID NO: 79. In embodiments, the antibody or antigen-binding fragment thereof comprises an amino acid sequence that is at least 80%, 85%, 90%, 95% or 100% identical to SEQ ID NO: 80. In embodiments, the antibody or antigen-binding fragment thereof comprises the amino acid sequence of SEQ ID NO: 80. In embodiments, the antibody or antigen-binding fragment thereof comprises an amino acid sequence that is at least 80%, 85%, 90%, 95% or 100% identical to SEQ ID NO: 83. In embodiments, the antibody or antigen-binding fragment thereof comprises the amino acid sequence of SEQ ID NO: 83. In embodiments, the antibody or antigen-binding fragment thereof comprises an amino acid sequence that is at least 80%, 85%, 90%, 95% or 100% identical to SEQ ID NO: 84. In embodiments, the antibody or antigen-binding fragment thereof comprises the amino acid sequence of SEQ ID NO: 84.

In embodiments, the antibody or antigen-binding fragment thereof comprises a V_{H} domain wherein the V_{H} domain comprises: a) the HCDR3 amino acid sequence of SEQ ID NO: 5, or SEQ ID NO: 5 which comprises 3, 2 or 1 amino acid substitution(s); and i. a HCDR1 amino acid sequence of SEQ ID NO: 3, or SEQ ID NO: 3 which comprises 3, 2 or 1 amino acid substitution(s); and/or ii. a HCDR2 amino acid sequence of SEQ ID NO: 4, or SEQ ID NO: 4 which comprises 3, 2 or 1 amino acid substitution(s); b) the HCDR3 amino acid sequence of SEQ ID NO: 5, or SEQ ID NO: 5 which comprises 3, 2 or 1 amino acid substitution(s); and i. a HCDR1 amino acid sequence of SEQ ID NO: 11, or SEQ ID NO: 11 which comprises 3, 2 or 1 amino acid substitution(s); and/or ii. a HCDR2 amino acid sequence of SEQ ID NO: 12, or SEQ ID NO: 12 which comprises 3, 2 or 1 amino acid substitution(s), c) the HCDR3 amino acid sequence of SEQ ID NO: 5, or SEQ ID NO: 5 which comprises 3, 2 or 1 amino acid substitution(s); and i. a HCDR1 amino acid sequence of SEQ ID NO: 14, or SEQ ID NO: 14 which comprises 3, 2 or 1 amino acid substitution(s); and/or ii. a HCDR2 amino acid sequence of SEQ ID NO: 15, or SEQ ID NO: 15 which comprises 3, 2 or 1 amino acid substitution(s), d) the HCDR3 amino acid sequence of SEQ ID NO: 5, or SEQ ID NO: 5 which comprises 3, 2 or 1 amino acid substitution(s); and i. a HCDR1 amino acid sequence of SEQ ID NO: 17, or SEQ ID NO: 17 which comprises 3, 2 or 1 amino acid substitution(s); and/or ii. a HCDR2 amino acid sequence of SEQ ID NO: 18, or SEQ ID NO: 18 which comprises 3, 2 or 1 amino acid substitution(s), e) the HCDR3 amino acid sequence of SEQ ID NO: 22, or SEQ ID NO: 22 which comprises 3, 2 or 1 amino acid substitution(s); and i. a HCDR1 amino acid sequence of SEQ ID NO: 20, or SEQ ID NO: 20 which comprises 3, 2 or 1 amino acid substitution(s); and/or ii. a HCDR2 amino acid sequence of SEQ ID NO: 21, or SEQ ID NO: 21 which comprises 3, 2 or 1 amino acid substitution(s), f) the HCDR3 amino acid sequence of SEQ ID NO: 30, or SEQ ID NO: 30 which comprises 3, 2 or 1 amino acid substitution(s); and i. a HCDR1 amino acid sequence of SEQ ID NO: 28, or SEQ ID NO: 28 which comprises 3, 2 or 1 amino acid substitution(s); and/or ii. a HCDR2 amino acid sequence of SEQ ID NO: 29, or SEQ ID NO: 29 which comprises 3, 2 or 1 amino acid substitution(s), g) the HCDR3 amino acid sequence is as defined by Chothia or Kabat and is from a V_{H} domain selected from SEQ ID NO: 2, or wherein the HCDR3 amino acid sequence is as defined by Chothia or Kabat and is from a V_{H} domain selected from SEQ ID NO: 2 and comprises 3, 2 or 1 amino acid substitution(s); and i. a HCDR1 amino acid sequence is as defined by Chothia or Kabat and is from a V_{H} domain selected from SEQ ID NO: 2, or wherein the HCDR1 amino acid sequence is as defined by Chothia or Kabat and is from a V_{H} domain selected from SEQ ID NO: 2 and comprises 3, 2 or 1 amino acid substitution(s); and/or ii. a HCDR2 amino acid sequence is as defined by Chothia or Kabat and is from a V_{H} domain selected from SEQ ID NO: 2, or wherein the HCDR2 amino acid sequence is as defined by Chothia or Kabat and is from a V_{H} domain selected from SEQ ID NO: 2 and comprises 3, 2 or 1 amino acid substitution(s), h) the HCDR3 amino acid sequence is as defined by Chothia or Kabat and is from a V_{H} domain selected from SEQ ID NO: 10, or wherein the HCDR3 amino acid sequence is as defined by Chothia or Kabat and is from a V_{H} domain selected from SEQ ID NO: 10 and comprises 3, 2 or 1 amino acid substitution(s); and i. a HCDR1 amino acid sequence is as defined by Chothia or Kabat and is from a V_{H} domain selected from SEQ ID NO: 10, or wherein the HCDR1 amino acid sequence is as defined by Chothia or Kabat and is from a V_{H} domain selected from SEQ ID NO: 10 and comprises 3, 2 or 1 amino acid substitution(s); and/or ii. a HCDR2 amino acid sequence is as defined by Chothia or Kabat and is from a V_{H} domain selected from SEQ ID NO: 10, or wherein the HCDR2 amino acid sequence is as defined by Chothia or Kabat and is from a V_{H} domain selected from SEQ ID NO: 10 and comprises 3, 2 or 1 amino acid substitution(s), i) the HCDR3 amino acid sequence is as defined by Chothia or Kabat and is from a V_{H} domain selected from SEQ ID NO: 13, or wherein the HCDR3 amino acid sequence is as defined by Chothia or Kabat and is from a V_{H} domain selected from SEQ ID NO: 13 and comprises 3, 2 or 1 amino acid substitution(s); and i. a HCDR1 amino acid sequence is as defined by Chothia or Kabat and is from a V_{H} domain selected from SEQ ID NO: 13, or wherein the HCDR1 amino acid sequence is as defined by Chothia or Kabat and is from a V_{H} domain selected from SEQ ID NO: 13 and comprises 3, 2 or 1 amino acid substitution(s); and/or ii. a HCDR2 amino acid sequence is as defined by Chothia or Kabat and is from a V_{H} domain selected from SEQ ID NO: 13, or wherein the HCDR2 amino acid sequence is as defined by Chothia or Kabat and is from a V_{H} domain selected from SEQ ID NO: 13 and comprises 3, 2 or 1 amino acid substitution(s), j) the HCDR3 amino acid sequence is as defined by Chothia or Kabat and is from a V_{H} domain selected from SEQ ID NO: 16, or wherein the HCDR3 amino acid sequence is as defined by Chothia or Kabat and is from a V_{H} domain selected from SEQ ID NO: 16 and comprises 3, 2 or 1 amino acid substitution(s); and i. a HCDR1 amino acid sequence is as defined by Chothia or Kabat and is from a V_{H} domain selected from SEQ ID NO: 16, or wherein the HCDR1 amino acid sequence is as defined by Chothia or Kabat and is from a V_{H} domain selected from SEQ ID NO: 16 and comprises 3, 2 or 1 amino acid substitution(s); and/or ii. a HCDR2 amino acid sequence is as defined by Chothia or Kabat and is from a V_{H} domain selected from SEQ ID NO: 16, or wherein the HCDR2 amino acid sequence is as defined by Chothia or Kabat and is from a V_{H} domain selected from SEQ ID NO: 16 and comprises 3, 2 or 1 amino acid substitution(s), k) the HCDR3 amino acid sequence is as defined by Chothia or Kabat and is from a V_{H} domain selected from SEQ ID NO: 19, or wherein the HCDR3 amino acid sequence is as defined by Chothia or Kabat and is from a V_{H} domain selected from SEQ ID NO: 19 and comprises 3, 2 or 1 amino acid substitution(s); and i. a HCDR1 amino acid sequence is as defined by Chothia or Kabat and is from a V_{H} domain selected from SEQ ID NO: 19, or wherein the HCDR1 amino acid sequence is as defined by Chothia or Kabat and is from a V_{H} domain selected from SEQ ID NO: 19 and comprises 3, 2 or 1 amino acid substitution(s); and/or ii. a HCDR2 amino acid sequence is as defined by Chothia or Kabat and is from a V_{H} domain selected from SEQ ID NO: 19, or wherein the HCDR2 amino acid sequence is as defined by Chothia or Kabat and is from a V_{H} domain selected from SEQ ID NO: 19 and comprises 3, 2 or 1 amino acid substitution(s), I) the HCDR3 amino acid sequence is as defined by Chothia or Kabat and is from a V_{H} domain selected from SEQ ID NO: 27, or wherein the HCDR3 amino acid sequence is as defined by Chothia or Kabat and is from a V_{H} domain selected from SEQ ID NO: 27 and comprises 3, 2 or 1 amino acid substitution(s); and i. a HCDR1 amino acid sequence is as defined by Chothia or Kabat and is from a V_{H} domain selected from SEQ ID NO: 27, or wherein the HCDR1 amino acid sequence is as defined by Chothia or Kabat and is from a V_{H} domain selected from SEQ ID NO: 27 and comprises 3, 2 or 1 amino acid substitution(s); and/or ii. a HCDR2 amino acid sequence is as defined by Chothia or Kabat and is from a V_{H} domain selected from SEQ ID NO: 27, or wherein the HCDR2 amino acid sequence is as defined by Chothia or Kabat and is from a V_{H} domain selected from SEQ ID NO: 27 and comprises 3, 2 or 1 amino acid substitution(s).

In embodiments, the antibody or antigen-binding fragment thereof comprises a V_{L} domain wherein the V_{L} domain comprises: a) the LCDR3 amino acid sequence of SEQ ID NO: 9, or SEQ ID NO: 9 which comprises 3, 2 or 1 amino acid substitution(s); and i. a LCDR1 amino acid sequence of SEQ ID NO: 7, or SEQ ID NO: 7 which comprises 3, 2 or 1 amino acid substitution(s); and/or ii. a LCDR2 amino acid sequence of SEQ ID NO: 8, or SEQ ID NO: 8 which comprises 3, 2 or 1 amino acid substitution(s); b) the LCDR3 amino acid sequence of SEQ ID NO: 26, or SEQ ID NO: 26 which comprises 3, 2 or 1 amino acid substitution(s); and i. a LCDR1 amino acid sequence of SEQ ID NO: 24, or SEQ ID NO: 24 which comprises 3, 2 or 1 amino acid substitution(s); and/or ii. a LCDR2 amino acid sequence of SEQ ID NO: 25, or SEQ ID NO: 25 which comprises 3, 2 or 1 amino acid substitution(s), c) the LCDR3 amino acid sequence of SEQ ID NO: 33, or SEQ ID NO: 33 which comprises 3, 2 or 1 amino acid substitution(s); and i. a LCDR1 amino acid sequence of SEQ ID NO: 32, or SEQ ID NO: 32 which comprises 3, 2 or 1 amino acid substitution(s); and/or ii. a LCDR2 amino acid sequence of SEQ ID NO: 25, or SEQ ID NO: 25 which comprises 3, 2 or 1 amino acid substitution(s), d) the LCDR3 amino acid sequence is as defined by Chothia or Kabat and is from a V_{L} domain selected from SEQ ID NO: 6, or wherein the LCDR3 amino acid sequence is as defined by Chothia or Kabat and is from a V_{L} domain selected from SEQ ID NO: 6 and comprises 3, 2 or 1 amino acid substitution(s); and i. a LCDR1 amino acid sequence is as defined by Chothia or Kabat and is from a V_{L} domain selected from SEQ ID NO: 6, or wherein the LCDR1 amino acid sequence is as defined by Chothia or Kabat and is from a V_{L} domain selected from SEQ ID NO: 6 and comprises 3, 2 or 1 amino acid substitution(s); and/or ii. a LCDR2 amino acid sequence is as defined by Chothia or Kabat and is from a V_{L} domain selected from SEQ ID NO: 6, or wherein the LCDR2 amino acid sequence is as defined by Chothia or Kabat and is from a V_{L} domain selected from SEQ ID NO: 6 and comprises 3, 2 or 1 amino acid substitution(s), e) the LCDR3 amino acid sequence is as defined by Chothia or Kabat and is from a V_{L} domain selected from SEQ ID NO: 23, or wherein the LCDR3 amino acid sequence is as defined by Chothia or Kabat and is from a V_{L} domain selected from SEQ ID NO: 23 and comprises 3, 2 or 1 amino acid substitution(s); and i. a LCDR1 amino acid sequence is as defined by Chothia or Kabat and is from a V_{L} domain selected from SEQ ID NO: 23, or wherein the LCDR1 amino acid sequence is as defined by Chothia or Kabat and is from a V_{L} domain selected from SEQ ID NO: 23 and comprises 3, 2 or 1 amino acid substitution(s); and/or ii. a LCDR2 amino acid sequence is as defined by Chothia or Kabat and is from a V_{L} domain selected from SEQ ID NO: 23, or wherein the LCDR2 amino acid sequence is as defined by Chothia or Kabat and is from a V_{L} domain selected from SEQ ID NO: 23 and comprises 3, 2 or 1 amino acid substitution(s), f) the LCDR3 amino acid sequence is as defined by Chothia or Kabat and is from a V_{L} domain selected from SEQ ID NO: 31, or wherein the LCDR3 amino acid sequence is as defined by Chothia or Kabat and is from a V_{L} domain selected from SEQ ID NO: 31 and comprises 3, 2 or 1 amino acid substitution(s); and i. a LCDR1 amino acid sequence is as defined by Chothia or Kabat and is from a V_{L} domain selected from SEQ ID NO: 31, or wherein the LCDR1 amino acid sequence is as defined by Chothia or Kabat and is from a V_{L} domain selected from SEQ ID NO: 31 and comprises 3, 2 or 1 amino acid substitution(s); and/or ii. a LCDR2 amino acid sequence is as defined by Chothia or Kabat and is from a V_{L} domain selected from SEQ ID NO: 31, or wherein the LCDR2 amino acid sequence is as defined by Chothia or Kabat and is from a V_{L} domain selected from SEQ ID NO: 31 and comprises 3, 2 or 1 amino acid substitution(s).

In a preferred embodiment, antibodies of the invention are isolated or purified.

In embodiments, the antibody or antigen-binding fragment thereof inhibits PAR2 peptide activation of PAR2. In embodiments, the antibody or antigen-binding fragment thereof specifically binds to and inhibits the activity of PAR2, wherein inhibiting PAR2 activity comprises binding to Segment1, Helix0/1, ECL3 of PAR2 receptor. In embodiments, the antibody or antigen-binding fragment thereof specifically binds to PAR2 and inhibits PAR2 activation, wherein inhibiting PAR2 activation comprises inhibiting PAR2 tethered ligand binding. In embodiments, the antibody or antigen-binding fragment thereof specifically binds to PAR2 and inhibits PAR2 activation, wherein inhibiting PAR2 activation comprises inhibiting cross-activation by PAR1 tethered ligand in PAR1-PAR2 heterodimers. In embodiments, the antibody or antigen-binding fragment thereof specifically binds to PAR2, and inhibits the binding of PAR2 activating peptide to PAR2. In embodiments, the antibodies provided herein able to inhibit protease cleavage mediated activation of PAR2 (e.g. by trypsin) and peptide mediated activation of PAR2 (e.g. by PAR2-AP or PAR1-AP).

In embodiments, the antibody or antigen-binding fragment thereof binds to an epitope that is identical to an epitope to which an antibody or fragment selected from clones Y022065, Y022870, Y022877, Y022883, Y022054 and/or Y021171 specifically bind.

In embodiments, the antibody or antigen-binding fragment thereof binds to an epitope, wherein the epitope to which the antibody or fragment binds is identified by hydrogen deuterium exchange (HDX) and/or by site-directed mutagenesis and flow cytometry.

In embodiments, the antibody or antigen-binding fragment thereof specifically binds to PAR2, and inhibits the binding of PAR2 activating peptide to PAR2, wherein the antibody or fragment inhibits PAR2 activating peptide mediated accumulation of inositol monophosphate (IP) with an IC₅₀ of from 1 to 100 nM, optionally wherein PAR2 peptide mediated accumulation of IP is determined using a PAR2 peptide stimulated IP signalling assay.

In embodiments, the antibody or antigen-binding fragment thereof inhibits trypsin mediated activation of PAR2. In embodiments, the antibody or antigen-binding fragment thereof inhibits trypsin mediated accumulation of IP with an IC₅₀ of from 1 to 300 nM; optionally wherein trypsin mediated accumulation of IP is determined using a trypsin stimulated IP signalling assay.

In embodiments, the antibody or antigen-binding fragment thereof inhibits PAR2 activating peptide mediated accumulation of inositol monophosphate (IP) with an IC₅₀ of from 1 to 100 nM, optionally wherein PAR2 peptide inhibition is determined using an HTRF assay.

In embodiments, the antibody or antigen-binding fragment thereof specifically binds to PAR2, wherein the antibody or fragment inhibits PAR2 activating peptide mediated calcium mobilisation, optionally with an IC₅₀ of from 1 to 100 nM, and optionally wherein calcium mobilisation is determined using a PAR2 activating peptide stimulated calcium mobilisation assay.

In embodiments, the antibody or antigen-binding fragment thereof inhibits trypsin mediated calcium mobilisation, optionally with an IC₅₀ of from 1 to 200 nM, and optionally wherein calcium mobilisation is determined using a PAR2 activating peptide stimulated calcium mobilisation assay.

In embodiments, the antibody or antigen-binding fragment thereof is not internalised into a cell upon binding to PAR2 on the surface of the cell, optionally wherein internalisation is determined by quantifying antibody or fragment binding using FACs.

In embodiments, the antibody or antigen-binding fragment thereof does not inhibit ligand SFLLR mediated PAR1 activation, wherein PAR1 activation is determined by using a ligand SFLLR stimulated IP signalling assay.

In embodiments, the antibody or antigen-binding fragment thereof binds to cynomolgus PAR2 with an EC₅₀ of from 600 pM to 5 nM or less, optionally wherein cynomolgus PAR2 binding is determined using flow cytometry.

In embodiments, the antibody or antigen-binding fragment thereof binds to human PAR2 with a K_{D} of 100 pM to 10 nM, optionally wherein binding affinity is determined using surface plasmon resonance (SPR) or KinExA.

In embodiments, the antibody or antigen-binding fragment thereof specifically binds to PAR2, and inhibits the binding of PAR2 peptide to PAR2, wherein binding of the antibody or fragment to PAR2 is pH independent between pH 7.5 and 6.0.

In embodiments, the antibody or antigen-binding fragment thereof does not bind to PAR1, optionally wherein PAR1 binding is determined using flow cytometry or ELISA. In embodiments, the antibody or antigen-binding fragment thereof does not bind to PAR3, optionally wherein PAR3 binding is determined using flow cytometry or ELISA. In embodiments, the antibody or antigen-binding fragment thereof does not bind to PAR4, optionally wherein PAR4 binding is determined using flow cytometry or ELISA.

In embodiments, the antibody or antigen-binding fragment thereof specifically binds to PAR2, and inhibits the binding of PAR2 peptide to PAR2, wherein 3 mg/kg antibody or fragment suppresses PAR2 stimulant induced response in leucocytes by >95% over a period of 30 days, wherein suppression is measured by determining stimulant induced gene signatures. In embodiments, the antibody or antigen-binding fragment thereof specifically binds to PAR2, and inhibits the binding of PAR2 peptide to PAR2, wherein 1 mg/kg antibody or fragment suppresses PAR2 peptide induced response in leucocytes by >90% over a period of 30 days, wherein suppression is measured by determining stimulant induced gene signatures.

In embodiments, the antibody or antigen-binding fragment thereof competes with functional ligand AZ8838 in binding to PAR2. In embodiments, the antibody or antigen-binding fragment thereof directly competes with AZ8838 in binding to PAR2.

In embodiments, the antibody or antigen-binding fragment thereof binds to PAR2 homodimers. In embodiments, the antibody or antigen-binding fragment thereof binds to PAR2-PAR1 heterodimers, optionally wherein binding inhibits cross activation of PAR2 by PAR1 tethered ligand.

Provided herein are antibodies and antigen-binding fragments thereof that bind PAR2 for use in therapy.

In embodiments, the antibody or antigen-binding fragment thereof is for use in treating a PAR2-mediated disease or condition e.g. atopic dermatitis, asthma, cancer (various including breast, melanoma, head and neck), pain (chronic, inflammatory, post-operative, neuropathic, fracture, gout, cancer, gastrointestinal associated with inflammatory bowel disease), rheumatoid arthritis and associated uveitis, scleroderma, systemic lupus erythematosus, osteoarthritis, polymyalgia rheumatica, ankylosing spondylitis, Reiter's disease, psoriatic arthritis, chronic Lyme arthritis, Still's disease, dermatomyositis, inclusion body myositis, polymyositis, and lymphangioleiomyomatosis.

In embodiments, the antibody or antigen-binding fragment thereof is for use in the manufacture of a medicament for treating a PAR2-mediated disease or condition e.g. atopic dermatitis, asthma, cancer (various including breast, melanoma, head and neck), pain (chronic, inflammatory, post-operative, neuropathic, fracture, gout, cancer, gastrointestinal associated with inflammatory bowel disease), rheumatoid arthritis and associated uveitis, scleroderma, systemic lupus erythematosus, osteoarthritis, polymyalgia rheumatica, ankylosing spondylitis, Reiter's disease, psoriatic arthritis, chronic Lyme arthritis, Still's disease, dermatomyositis, inclusion body myositis, polymyositis, and lymphangioleiomyomatosis.

Provided herein is a method of treating a PAR2-mediated disease or condition (e.g. pain, optionally wherein the pain is independently selected from chronic pain, inflammatory pain, post-operative pain, neuropathic pain, fracture associated pain, gout associated pain, cancer associated pain, gastrointestinal pain associated with inflammatory bowel disease etc.) in a patient, comprising administering to said patient (e.g. human) a therapeutically effective amount of an antibody or fragment thereof of the disclosure, wherein the PAR2-mediated disease or condition is thereby treated.

In embodiments, the antibodies of the disclosure may be combined or administered alongside a further therapy, optionally wherein the further therapy comprises one or more further therapeutic agent(s) independently selected from the group consisting of analgesics including anti-inflammatory drugs (e.g. NSAIDS including aspirin, ibuprofen, diclofenac, naproxen), paracetamol, opioids (e.g. codeine, morphine, oxycodone, fentanyl, buprenorphine), amitriptyline, gabapentin; anticancer drugs including alkylating agents (e.g. nitrogen mustards, nitrourea), antimetabolites (e.g. folic acid analogues, pyrimidine and purine analogues), antibiotics and enzymes (e.g. dactinomycin, daunorubicin, doxorubicin, L-asparaginase), natural agents (e.g. vinca alkaloids, taxens, tecans), hormones and antagonists (e.g. progestins, estrogen, GnRH, anti-estrogens), hyroxyurea, immunomodulators, tyrosine kinase inhibitors, biological response modifiers, molecularly targeted therapies (e.g. antibody conjugated drugs), platinum based therapies (e.g. cisplatin, carboplatin, oxaliplatin); and/or optionally wherein the further therapy is selected from radiotherapy and/or surgical removal of tumours.

Provided herein is a pharmaceutical composition comprising an antibody or fragment of the disclosure and a pharmaceutically acceptable excipient, diluent or carrier and optionally further comprising one or more further therapeutic agents independently selected from the group consisting of analgesics including anti-inflammatory drugs (e.g. NSAIDS including aspirin, ibuprofen, diclofenac, naproxen), paracetamol, opioids (e.g. codeine, morphine, oxycodone, fentanyl, buprenorphine), amitriptyline, gabapentin; anticancer drugs including alkylating agents (e.g. nitrogen mustards, nitrourea), antimetabolites (e.g. folic acid analogues, pyrimidine and purine analogues), antibiotics and enzymes (e.g. dactinomycin, daunorubicin, doxorubicin, L-asparaginase), natural agents (e.g. vinca alkaloids, taxens, tecans), hormones and antagonists (e.g. progestins, estrogen, GnRH, anti-estrogens), hyroxyurea, immunomodulators, tyrosine kinase inhibitors, biological response modifiers, molecularly targeted therapies (e.g. antibody conjugated drugs), platinum based therapies (e.g. cisplatin, carboplatin, oxaliplatin).

Provided herein is a pharmaceutical composition of the disclosure or a kit comprising said pharmaceutical composition, wherein the composition is for treating a PAR2 mediated disease or condition, e.g. selected from atopic dermatitis, asthma, cancer (various including breast, melanoma, head and neck), pain (chronic, inflammatory, post-operative, neuropathic, fracture, gout, cancer, gastrointestinal associated with inflammatory bowel disease), rheumatoid arthritis and associated uveitis, scleroderma, systemic lupus erythematosus, osteoarthritis, polymyalgia rheumatica, ankylosing spondylitis, Reiter's disease, psoriatic arthritis, chronic Lyme arthritis, Still's disease, dermatomyositis, inclusion body myositis, polymyositis, and lymphangioleiomyomatosis.

Provided herein is a pharmaceutical composition of the disclosure, or a kit of the disclosure in combination with a label or instructions for use to treat a disease or condition in a patient; optionally wherein the label or instructions comprise a marketing authorisation number (e.g., an FDA or EMA authorisation number); optionally wherein the kit comprises an IV or injection device that comprises said antibody or fragment.

In embodiments, the amino acid substitution(s) comprise homologous substitution(s). These amino acid substitution(s) may be conservative substitution(s). Conservative amino acid substitution(s) refer to substitutions of an amino acid residue with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In some aspects, a predicted nonessential amino acid residue in an anti-PAR-2 antibody is replaced with another amino acid residue from the same side chain family. Methods of identifying amino acid conservative substitutions which do not eliminate antigen binding are well-known in the art (see, e.g., Brummell et al., Biochem. 32:1180-1187 (1993); Kobayashi et al. Protein Eng. 12 (10): 879-884 (1999); and Burks et al. Proc. Natl. Acad. Sci. USA 94:412-417 (1997)).

In embodiments, the amino acid substitution(s) reduce the binding affinity of the antibody or antigen-binding fragment thereof for human PAR2 by no more than 1000, 800, 700, 500, 400, 300, 200, 100, 50 or 10-fold as compared to an antibody or antigen-binding fragment having a VH with an amino acid sequence of SEQ ID NO: 16 and VL with an amino acid sequence of SEQ ID NO: 6 when tested in a PAR2 binding assay, such as an SPR or Kinexa assay, at a pH of between 7.4 and 7.6.

The antibodies provided herein may be human. In embodiments, the antibody is a monoclonal antibody. In embodiments, the antibody is an IgG antibody. In embodiments, the antibody or antigen-binding fragment thereof is an antigen-binding fragment. In embodiments, the antigen binding fragment is a scFv. In embodiments, the antigen-binding fragment is a Fab. In embodiments, the antibody or antigen-binding fragment thereof is humanized.

The antibodies provided herein bind to human PAR2, and may also bind to Cynomolgus PAR2 and Rhesus PAR2 but not to mouse and/or rat PAR2.

In embodiments, the antibody or antigen-binding fragment thereof prevents trypsin, tryptase and/or matriptase from interacting with PAR2. In embodiments the antibody or antigen binding fragment thereof inhibits PAR2 activation by trypsin. In embodiments, the antibody or antigen-binding fragment thereof inhibits exposure of the tethered ligand. In embodiments, the antibody or antigen-binding fragment thereof prevents the tethered ligand from interacting with PAR2.

In embodiments, provided herein are nucleic acids capable of expressing the antibodies or antigen-binding fragments thereof. In embodiments, the nucleic acid comprises a nucleotide sequence that is at least 90% identical to any one of SEQ ID NO: 34, 70 and 72. In embodiments, the nucleic acid comprises a nucleotide sequence that is at least 95% identical to any one of SEQ ID NO: 34, 70 and 72. In embodiments, the nucleic acid comprises the nucleotide sequence of any one of SEQ ID NO: 34, 70 and 72. In embodiments, the nucleic acid comprises a nucleotide sequence that is at least 90% identical to any one of SEQ ID NO: 35, 36, 37, 38, 71 and 73. In embodiments, the nucleic acid comprises a nucleotide sequence that is at least 95% identical to any one of SEQ ID NO: 35, 36, 37, 38, 71 and 73. In embodiments, the nucleic acid comprises the nucleotide sequence of any one of SEQ ID NO: 35, 36, 37, 38, 71 and 73. In embodiments, the nucleic acid comprises a nucleotide sequence that is at least 90% identical to SEQ ID NO: 34. In embodiments, the nucleic acid comprises a nucleotide sequence that is at least 95% identical to SEQ ID NO: 34. In embodiments, the nucleic acid comprises the nucleotide sequence of SEQ ID NO: 34. In embodiments, the nucleic acid comprises a nucleotide sequence that is at least 90% identical to SEQ ID NO: 38. In embodiments, the nucleic acid comprises a nucleotide sequence that is at least 95% identical to SEQ ID NO: 38. In embodiments, the nucleic acid comprises the nucleotide sequence of SEQ ID NO: 38.

In a preferred embodiment, nucleic acids of the invention are isolated or purified.

In embodiments, the disclosure provides for a vector comprising any of the nucleic acids disclosed herein. In embodiments, the disclosure provides for a set of vectors comprising any one or more of the nucleic acids disclosed herein.

In embodiments, the disclosure provides for a host cell comprising any one or more of the vectors disclosed herein.

In embodiments, the disclosure provides for a composition comprising a pharmaceutically acceptable carrier and any of the antibodies or antigen-binding fragments disclosed herein. In embodiments, the disclosure provides for a lyophilized composition comprising any of the antibodies or antigen-binding fragments thereof disclosed herein.

In embodiments, the disclosure provides for a reconstituted lyophilized composition comprising any of the antibodies or antigen-binding fragments thereof disclosed herein. In embodiments, the composition is formulated for administration by lozenge, spray, oral administration, delayed release or sustained release, trans-mucosal administration, syrup, mucoadhesive, buccal formulation, mucoadhesive tablet, topical administration, parenteral administration, injection, subdermal administration, oral solution, rectal administration, buccal administration or transdermal administration.

In embodiments, the disclosure provides for a kit comprising any of the antibodies or antigen-binding fragments disclosed herein or any of the compositions disclosed herein.

In embodiments, the disclosure provides for a method for treating pain in a subject in need thereof, comprising administering to the subject a pharmaceutically effective amount of any of the compositions disclosed herein. In some embodiments, the pain is selected from the group consisting of: nociceptive, neuropathic, and mix-type pain. In some embodiments, the pain is associated with a headache, chronic headache, a migraine headache, a cancer, a viral infection, rheumatoid arthritis, osteoarthritis, Crohn's disease, liver disease, multiple sclerosis, spinal cord injury, post herpetic neuralgia, diabetic neuropathy, lower back pain, inflammatory heart disease, kidney disease, gastritis, gingivitis, periodontal disease, asthma, chronic obstructive pulmonary disease, autoimmune disease, irritable bowel syndrome, fibromyalgia, leg pains, restless leg syndrome, diabetic neuropathy, an allergic condition, a surgical procedure, acute or chronic physical injury, bone fracture or a crush injury, spinal cord injury, an inflammatory disease, a non-inflammatory neuropathic or dysfunctional pain condition, or a combination thereof. In some embodiments, the pain is osteoarthritis pain. In some embodiments, the subject is a human.

In some embodiments, the disclosure provides for a method of producing any of the antibodies or antigen-binding fragments disclosed herein, comprising the steps of: expressing any of the nucleic acids disclosed herein in a cultured cell, purifying the antibody or antigen-binding fragment.

### DETAILED DESCRIPTION OF THE INVENTION

The present invention provides antibodies or antigen-binding fragments that bind to human PAR2 receptor. The antibodies of the present invention are useful for inhibiting PAR2 and its downstream signalling cascade.

Before the present disclosure is described, it is to be understood that this disclosure is not limited to particular methods and experimental conditions described, as such methods and conditions may vary. It is also to be understood that the terminology used herein is for the purpose of describing particular embodiments only, and is not intended to be limiting.

PAR2 or GPR11 (PAR2; human PAR2 Uniprot Protein ID: P55085) is a 44 kDa G protein-coupled receptor that belongs to a family of Protease-Activated Receptors (PAR). It is encoded by the gene F2RL1 (coagulation factor II receptor-like 1). PAR2 belongs to a family of Protease-Activated Receptors (PAR), which are activated by proteolytic cleavage within the extracellular N-terminus. This family comprises four members PAR1-PAR4 that are activated by different proteases. PAR2 is predominantly activated by the serine proteases tryptase and trypsin, while other PAR family members are mostly activated by thrombin, although proteinase 3, factor VIIa and factor Xa are also described to be involved in PAR activation. For reviews, see Yau et al., 2016; Mrozkova et al., 2016; Hamilton and Trejo, 2017; Kagota et al., 2016.

PAR2 is activated by three main mechanisms. One of the mechanisms involves the cleavage of the extracellular N-terminal domain by proteases. This results in the exposure of a tethered ligand, which binds to a conserved region on extracellular loop 2 on the receptor and triggers intracellular signalling. Alternatively, PAR2 can be activated by a synthetic short peptide known as activated peptide (PAR2-AP) that mimics the first six amino acids of the tethered N-terminal ligand. Additionally, PAR2 can also be activated by cross-activation by PAR1 tethered ligand in PAR1-PAR2 hetero-dimerization. Upon activation of PAR2, G_{oq} and Gᵢ proteins are activated which in turn results in an influx of calcium, induction of MAPK signalling and downstream inflammatory signalling. This results in subsequent biological responses, such as proliferation or secretion of pro-inflammatory cytokines, e.g., IL-6, IL-8 (also known as CXCL8) and GM-CSF.

PAR2 expression has been shown to be increased in synovial lining, chondrocytes, and tissues in human rheumatoid arthritis and animal models of arthritis (Amiable et al 2009). PAR2 also potentiates signalling via channels such as TRPV1 (Dai et al 2007), a ligand-gated ion channel involved in inflammatory pain. PAR2 signalling is also known to sensitize TRPV1 in vivo, resulting in thermal hyperalgesia (Amadesi et al., 2006).

PAR2 activation has been shown to be responsible for various inflammatory signalling pathways. In mice lacking the PAR2 receptor, there is a delayed onset of inflammation in response to inflammatory mediators (Lindner et al, 2000). Other rodent PAR2 knockout studies have demonstrated that PAR2 plays an important role in pathophysiology of many disease conditions such as pain, musculoskeletal inflammation including osteoarthritis, neuro-inflammatory disorders, airway inflammation, itch, dermatitis, colitis and related conditions (Yau et al 2013). PAR2 receptor antagonists such as GB88 have also been shown to block inflammatory responses in vivo including the collagen-induced arthritis model in rats (Lohman et al 2012).

Known small molecule PAR2 antagonists are not ideal therapeutics due to modest potency and broad specificity. Given the range of diseases in which PAR2 is considered pivotal there is a need to identify a potent, specific PAR2 antagonist. The antibodies of the present invention are potent and specific PAR2 antagonists that inhibit PAR2 activation mediated by cleavage of the N-terminal domain.

Unless defined otherwise, all technical and scientific terms used herein have the same meaning as commonly understood by one of ordinary skill in the art to which this disclosure belongs.

Although any methods and materials similar or equivalent to those described herein can be used in the practice or testing of the present disclosure, the preferred methods and materials are now described.

As used in this specification and the appended claims, the singular form “a”, “an” and “the” include plural referents unless the context clearly dictates otherwise.

It is convenient to point out here that “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

All numerical designations, e.g., pH, temperature, time, concentration, and molecular weight, including ranges, are approximations. It is to be understood, although not always explicitly stated that all numerical designations are preceded by the term “about.” It also is to be understood, although not always explicitly stated, that the reagents described herein are merely examples and that equivalents of such are known in the art.

The terms “Human protease activated receptor 2,” or “human PAR2,” or “PAR2,” and the like, as used herein, refer to human PAR2 (wildtype or wt) with UniProt ID number: P55085, reproduced herein as SEQ ID NO: 1. Human PAR2 includes any sequence that is at least 99% or 100% identical to the amino acid sequence of any of SEQ ID NO: 1, or biologically active fragments thereof.

The term “tethered ligand”, as used herein, refers to a region of the N-terminal portion of PAR2 that is exposed following proteolytic cleavage by a protease (e.g., trypsin) and, once cleaved, binds to a proximal binding site on the PAR2 receptor to activate it. The tethered ligand at the N-terminus of the human PAR2 receptor comprises SEQ ID NO: 39.

By “PAR2-AP,” “PAR2 activated peptide” or “PAR2 activating peptide,” it is meant a synthetic short peptide which mimics the cleaved tethered N-terminal ligand. The human peptide is SEQ ID NO: 39 and the murine peptide comprises SEQ ID NO: 40.

The term “antibody,” “antibody to PAR2” or “anti-PAR2,” as used herein, refers to whole antibodies or antigen-binding fragments. Antibodies, as used herein, interact with (e.g., by binding, steric hindrance, stabilizing/destabilizing, spatial distribution) PAR2 and interfere with activation of PAR2. A naturally occurring “antibody” is a glycoprotein comprising at least two heavy (H) chains and two light (L) chains inter-connected by disulfide bonds. The heavy chains comprise a variable region (VH) and a constant region, and the light chains comprise a variable region (VL) and a constant region. The VH and VL regions can be further divided into hypervariable (HV) and framework (FR) regions. Each VH and VL is composed of three complementarity determining regions (CDRs) and four FRs. The term “antibody” may refer, for example, to monoclonal antibodies, human antibodies, humanized antibodies, shark antibodies, camelid antibodies, or chimeric antibodies.

Both the light and heavy chains are divided into regions of structural and functional homology. The terms “constant” and “variable” are used functionally. In this regard, it will be appreciated that the variable domains of both the heavy and light chain determine antigen recognition and specificity. Conversely, the constant domains of the light chain (CL) and the heavy chain (CH1, CH2 or CH3) confer important biological properties such as secretion, trans-placental mobility, Fc receptor binding, complement binding, and the like. By convention the numbering of the constant region domains increases as they become more distal from the antigen-binding site or amino-terminus of the antibody. The N-terminus is a variable region and at the C-terminus is a constant region; the CH3 and CL domains actually comprise the carboxy-terminus of the heavy and light chain, respectively.

Depending on the amino acid sequences of the constant domains of their heavy chains, antibodies (immunoglobulins) can be assigned to different classes. There are five major classes (isotypes) of immunoglobulins in humans: IgA, IgD, IgE, IgG, and IgM, and several of these may be further divided into subclasses, e.g., IgG₁, IgG₂, IgG₃, IgG₄, IgA₁, and IgA₂. The heavy chain constant domains that correspond to the different classes of immunoglobulins are called α, δ, ε, γ, and μ, respectively. The subunit structures and three-dimensional configurations of different classes of immunoglobulins are well known and described generally in, for example, Abbas et al., (2000). An antibody may be part of a larger fusion molecule, formed by covalent or non-covalent association of the antibody with one or more other proteins or peptides.

The “Complementarity Determining Regions” (“CDRs”) are amino acid sequences with boundaries determined using any of a number of well-known schemes such as the “Kabat” and “Chothia” numbering scheme as shown in Table 1. For example, under the Kabat scheme, the CDR amino acid residues in the heavy chain the CDR amino acid residues in the heavy chain variable domain (VH) are numbered 31-35 (HCDR1), 50-65 (HCDR2), and 95-102 (HCDR3); whereas under Chothia the CDR amino acids in the V_{H} are numbered 26-32 (HCDR1), 52-56 (HCDR2), and 95-102 (HCDR3).

### Table 1: CDR Definitions (Numbering is According to the Kabat Nomenclature).

| | Chothia | Kabat |
|---|---|---|
| CDR-L1 | L26-L32 | K24-L34 |
| CDR-L2 | L50-L52 | L50-L56 |
| CDR-L3 | L91-L96 | L89-L97 |
| CDR-H1 | H26-H32 | H31-H35 |
| CDR-H2 | H52-H56 | H50-K65 |
| CDR-H3 | H95-H102 | H95-H102 |

The terms “antigen-binding fragment” or “epitope-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Although the two fragment variable (Fv) domains, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., 1988; and Huston et al., 1988). Such single chain antibodies are also intended to be encompassed within the terms “fragment”, “epitope-binding fragment” or “antibody fragment”. These fragments are obtained using conventional techniques known to those of skill in the art, and the fragments are screened for utility in the same manner as are intact antibodies.

Further non-limiting examples of antigen-binding fragments include: (i) Fab fragments; (ii) Fab′ fragments; (iii) F(ab′)2 fragments; (iv) Fd fragments; (v) Fv fragments; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, camelid antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), adnectins, small modular immune-pharmaceuticals (SMIPs), and shark variable IgNAR domains, are also encompassed within the expression “antigen-binding fragment,” as used herein.

An antigen-binding fragment of an antibody will typically comprise at least one variable domain (e.g., at least one of a VH or VL). The variable domain may be of any size or amino acid composition and will generally comprise at least one CDR which is adjacent to or in frame with one or more framework sequences. In antigen-binding fragments having a VH domain associated with a VL domain, the VH and VL domains may be situated relative to one another in any suitable arrangement. For example, the variable region may be dimeric and contain VH-VH, VH-VL or VL-VL dimers. Alternatively, the antigen-binding fragment of an antibody may contain a monomeric VH or VL domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) VH-CH1; (ii) VH-CH2; (iii) VH-CH3; (iv) VH-CH1-CH2; (V) VH-CH1-CH2-CH3; (vi) VH-CH2-CH3; (vii) VH-CL; (viii) VL-CH1; (ix) VL-CH2; (x) VL-CH3; (xi) VL-CH1-CH2; (xii) VL-CH1-CH2-CH3; (xiii) VL-CH2-CH3; and (xiv) VL-CL. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. In some embodiments, the hinge region comprises a glycine-serine linker.

Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric VH or VL domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format may be adapted for use in the context of an antigen-binding fragment of an antibody of the present disclosure using routine techniques available in the art.

The term “biparatopic antibody” as used herein, refers to a bispecific antibody that binds to two different epitopes on a single PAR2 target.

The term “monovalent antibody” as used herein, refers to an antibody that comprises one epitope-binding moiety. The term “bivalent antibody” as used herein, refers to an antibody that comprises two epitope-binding moieties.

The term “multivalent antibody” refers to a single binding molecule with more than one valency, where “valency” is described as the number of antigen-binding moieties present per molecule of an antibody construct. As such, the single binding molecule can bind to more than one binding site on a target molecule. Examples of multivalent antibodies include, but are not limited to bivalent antibodies, trivalent antibodies, tetravalent antibodies, pentavalent antibodies, and the like, as well as multispecific antibodies and biparatopic antibodies. For example, for PAR2, the multivalent antibody (e.g., a PAR2 biparatopic antibody) has two different binding moieties for two different epitopes of PAR2, respectively.

The terms “monoclonal antibody” or “mAb,” as used herein, refer to polypeptides, including antibodies, bispecific antibodies, etc. that have substantially identical amino acid sequences or are derived from the same genetic source. This term also includes preparations of antibody molecules of single molecular composition. A monoclonal antibody composition displays a single binding specificity and affinity for a particular epitope.

The term “fragment crystallisable region” or “Fc region,” as used herein, refers to a polypeptide comprising the CH3, CH2 and at least a portion of the hinge region of a constant domain of an antibody. Optionally, an Fc region may include a CH4 domain, present in some antibody classes. An Fc region may comprise the entire hinge region of a constant domain of an antibody. In one embodiment, a constant region is modified compared to a wild-type constant region. That is, the polypeptides of the invention disclosed herein may comprise alterations or modifications to one or more of the three heavy chain constant domains (CH1, CH2 or CH3) and/or to the light chain constant region domain (CL). Example modifications include additions, deletions or substitutions of one or more amino acids in one or more domains. Such changes may be included to optimize effector function, half-life, etc.

The terms “dual-active”, “dual-activity” or “dual-active antibody”, as used herein, refer to Abs, as provided herein, that are able to inhibit both protease cleavage mediated activation of PAR2 (e.g. by trypsin) and peptide mediated activation of PAR2 (e.g. by PAR2-AP or PAR1-AP).

The term “reference antibody,” “reference mAb” or “reference Ab or “benchmark” or “benchmark antibody,” as used herein, refers to any antibody used in this disclosure during experimentation for reference against the anti-PAR2 antibodies of the present invention (e.g. for positive or negative controls and setting up assay conditions). Benchmark antibodies used in the experiments herein include: Benchmark 1 which binds to an N-terminal epitope of PAR2 (Giblin et al 2011); Benchmark 2 (WO2018167322A1), also referred to as R001053 or PaB670129; Benchmark 3, a Regeneron Ab, also referred to as R001044 or H4H581P; Benchmark 4, an Amgen Ab, also referred to as R001048 or 1A1; MAB3949, a murine R&D systems mAb; and Benchmark 6 (Giblin et al 2011).

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational (e.g. discontinuous) or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

The term “binding site” as used herein comprises an area on PAR2 target molecule to which an antibody or antigen-binding fragment selectively binds.

Generally, antibodies specific for a particular target antigen will bind to an epitope on the target antigen in a complex mixture of proteins and/or macromolecules.

As used herein, the term “affinity” refers to the strength of interaction between antibody and antigen at single antigenic sites. Within each antigenic site, the variable region of the antibody “arm” interacts through weak non-covalent forces with the antigen at numerous sites; the more interactions, the stronger the affinity. As used herein, the term “high affinity” for an IgG antibody or fragment thereof (e.g., a Fab fragment) refers to an antibody having a KD of 10⁻⁸ M or less, 10⁻⁹ M or less, or 10⁻¹⁰ M, or 10⁻¹¹ M or less, or 10⁻¹² M or less, or 10⁻¹³ M or less for a target antigen. However, high affinity binding can vary for other antibody isotypes. For example, high affinity binding for an IgM isotype refers to an antibody having a KD of 10⁻⁷ M or less, or 10⁻⁸ M or less.

As used herein, the term “avidity” refers to an informative measure of the overall stability or strength of the antibody-antigen complex. It is controlled by three major factors: antibody epitope affinity; the valency of both the antigen and antibody; and the structural arrangement of the interacting parts. Ultimately these factors define the specificity of the antibody, that is, the likelihood that the particular antibody is binding to a precise antigen epitope.

The terms “nucleic acid” and “polynucleotide” are used interchangeably and refer to a polymeric form of nucleotides of any length, either deoxyribonucleotides or ribonucleotides or analogues thereof. Polynucleotides can have any three-dimensional structure and can perform any function. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, siRNAs, shRNAs, RNAi agents, and primers. A polynucleotide can be modified or substituted at one or more base, sugar and/or phosphate, with any of various modifications or substitutions described herein or known in the art. A polynucleotide can comprise modified nucleotides, such as methylated nucleotides and nucleotide analogues. If present, modifications to the nucleotide structure can be imparted before or after assembly of the polymer. The sequence of nucleotides can be interrupted by non-nucleotide components. A polynucleotide can be further modified after polymerization, such as by conjugation with a labelling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A “gene” refers to a polynucleotide containing at least one open reading frame (ORF) that is capable of encoding a particular polypeptide or protein after being transcribed and translated. A polynucleotide sequence can be used to identify larger fragments or full-length coding sequences of the gene with which they are associated. Methods of isolating larger fragment sequences are known to those of skill in the art.

As used herein the term “amino acid” refers to natural and/or unnatural or synthetic amino acids, both the D and L optical isomers of any amino acid and amino acid analogues. A peptide of three or more amino acids is commonly called an oligopeptide if the peptide chain is short. If the peptide chain is long, the peptide is commonly called a polypeptide or a protein. The terms “biomarker” or “marker” are used interchangeably herein. A biomarker is a nucleic acid, polypeptide or other organic or inorganic molecule expressed in humans and the presence or absence of a mutation or differential expression of the biomarker is used to determine sensitivity to any treatment comprising an anti-PAR2 antibody according to the invention. For example, a protein is a biomarker for a cancer cell when it is deficient, mutated, deleted, or decreased in post-translational modification, production, expression, level, stability and/or activity, as compared to the same protein in a normal (non-cancerous) cell or control cell.

The terms “polypeptide,” “peptide,” “peptidomimetic” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. The residues may be linked by peptide bonds or other bonds, e.g., ester, ether, etc.

A polynucleotide or polynucleotide region (or a polypeptide or polypeptide region) has a certain percentage (for example, 80%, 85%, 90%, 95%, 98% or 99%) of “sequence identity” to another sequence meaning that, when aligned, that percentage of bases (or amino acids) are the same in comparing the two sequences. This alignment and the percentage homology or sequence identity can be determined using software programs known in the art, for example, those described in Ausubel et al., (1987). Preferably, default parameters are used for alignment. Preferred alignment tools are provided on the European Molecular Biology Laboratory—European Bioinformatics Institute (EMBL-EBI) webpage, using default parameters. Other programs include BLAST, BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant.

The terms “expression product” or “gene product”, as used herein, refer to the nucleic acids or amino acids (e.g., peptide or polypeptide) generated when a gene is transcribed and translated.

As used herein, “expression” refers to the process by which DNA is transcribed into mRNA and/or the process by which the transcribed mRNA is subsequently translated into peptides, polypeptides or proteins. If the polynucleotide is derived from genomic DNA, expression may include splicing of the mRNA in a eukaryotic cell.

“Differentially expressed” as applied to a gene, refers to the differential production of the mRNA transcribed from the gene or the protein product encoded by the gene. A differentially expressed gene may be overexpressed or underexpressed as compared to the expression level of a normal or control cell. However, as used herein, overexpression is an increase in gene expression and generally is at least 1.25 fold or, alternatively, at least 1.5 fold or, alternatively, at least 2 fold, or alternatively, at least 3 fold or alternatively, at least 4 fold expression over that detected in a normal or control counterpart cell or tissue. As used herein, under expression, is a reduction of gene expression and generally is at least 1.25 fold, or alternatively, at least 1.5 fold, or alternatively, at least 2 fold or alternatively, at least 3 fold or alternatively, at least 4 fold expression under that detected in a normal or control counterpart cell or tissue. The term “differentially expressed” also refers to where expression in a cancer cell or cancerous tissue is detected but expression in a control cell or normal tissue (e.g. non-cancerous cell or tissue) is undetectable.

A high expression level of the gene can occur because of over expression of the gene or an increase in gene copy number. The gene can also be transcribed and translated into increased protein levels because of deregulation or absence of a negative regulator. Lastly, high expression of the gene can occur due to increased stabilization or reduced degradation of the protein, resulting in accumulation of the protein.

As used herein, the terms “inhibit” or “inhibiting” and the like refer to the interfering, antagonising or blocking of PAR2 receptor activation. Such terms denote quantitative differences between two states, e.g., refer to statistically significant differences between the two states. For example, “an amount effective to inhibit PAR2 activation” means that the activation of PAR2 due to agonist and PAR2 blocking antibody will be at least statistically significantly different from the cells treated with agonist alone.

The terms “treatment”, “treating”, “alleviation” and the like are used herein to generally mean obtaining a desired pharmacologic and/or physiologic effect, and may also be used to refer to improving, alleviating, and/or decreasing the severity of one or more symptoms of a condition being treated. The effect may be prophylactic in terms of completely or partially delaying the onset or recurrence of a disease, condition, or symptoms thereof, and/or may be therapeutic in terms of a partial or complete cure for a disease or condition and/or adverse effect attributable to the disease or condition. “Treatment” as used herein covers any treatment of a disease or condition of a mammal, particularly a human, and includes any one or more of: (a) preventing the disease or condition from occurring in a subject which may be predisposed to the disease or condition but has not yet been diagnosed as having it; (b) inhibiting the disease or condition (e.g., arresting its development); or (c) relieving the disease or condition (e.g., causing regression of the disease or condition, providing improvement in one or more symptoms). For example, “treatment” of pain (e.g., chronic or neuropathic pain) involves a reduction, arrest, alleviation, or elimination of pain symptoms in the treated subject. The population of subjects treated by the method of the disease includes subjects suffering from the undesirable condition or disease, as well as subjects at risk for development of the condition or disease.

By the term “therapeutically effective dose,” what is meant is a dose that produces the desired effect for which it is administered. The exact dose will depend on the purpose of the treatment, and will be ascertainable by one skilled in the art using known techniques (see, e.g., The Art, Science, and Technology of Pharmaceutical Compounding, 3rd Edition, 2008).

Once the nucleotide sequences encoding such antibodies have been determined, chimeric antibodies may be produced by recombinant methods. Nucleic acids encoding the antibodies are introduced into host cells and expressed using materials and procedures generally known in the art, and as disclosed herein.

An “isolated” or “purified” antibody or protein is one that has been identified, separated and/or recovered from a component of its production environment (e.g. natural or recombinant). For example, the antibody or protein is substantially free of cellular material or other contaminating proteins from the cell or tissue source from which the antibody is derived, or substantially free of chemical precursors or other chemicals when chemically synthesized. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 20%, 10%, or 5% (by dry weight) of heterologous protein (also referred to herein as a “contaminating protein”). When the antibody is recombinantly produced, it is also preferably substantially free of culture medium, i.e. culture medium represents less than about 20%, 10%, or 5% of the volume of the protein preparation. When the antibody is produced by chemical synthesis, it is preferably substantially free of chemical precursors or other chemicals, i.e., it is separated from chemical precursors or other chemicals which are involved in the synthesis of the protein.

Accordingly, such preparations of the antibody have less than about 30%, 20%, 10%, or 5% (by dry weight) of chemical precursors or compounds other than the antibody of interest. In a preferred embodiment, antibodies of the invention are isolated or purified.

Provided herein are PAR2-directed antibodies and antigen-binding fragments thereof that bind PAR2. In embodiments, the antibody is an antagonising, neutralizing and/or blocking anti-PAR2 antibody or antigen-binding fragment. An “antagonising”, “neutralizing” or “blocking” antibody or antigen-binding fragment, as used herein, is intended to refer to an antibody or antigen-binding fragment whose binding to PAR2: (i) inhibits the binding of PAR2 activating peptide to PAR2 or the activation of PAR2 by PAR2 activating peptide; and/or (ii) interferes with the interaction between PAR2 exposed tethered ligand and PAR2; and/or (iii) interferes with the interaction between PAR2 and a protease (e.g., trypsin, tryptase, matriptase, legumain); and/or (iv) inhibits PAR2 signalling (e.g. inhibits PAR2 mediated IP accumulation or intracellular Ca²⁺ mobilisation); and/or (vi) inhibits PAR2 activation; and/or (vii) results in inhibition of at least one biological function of PAR2 (e.g. PAR2 mediated inflammatory responses) and/or (viii) inhibits PAR2 secondary messenger signalling driven through activation of dominant Gαq signalling partner. In embodiments, the antibodies or antigen-binding fragments of the disclosure inhibit activation of PAR2. In some embodiments, the antibodies or antigen-binding fragments inhibit exposure of the tethered ligand. In some embodiments, the antibodies or antigen-binding fragments inhibit activation of a PAR2 receptor by its exposed tethered ligand. In embodiments, the antibodies or antigen-binding fragments inhibit activation by the exposed tethered ligand of PAR2. In embodiments, the antibodies or antigen-binding fragments inhibit binding of the exposed tethered ligand to the second extracellular loop (ECL2) of PAR2. In embodiments, the antibodies or antigen binding fragments thereof bind to a discontinuous epitope, wherein binding to said epitope occludes binding of the exposed tethered ligand to ECL2 via steric hindrance. The inhibition caused by an anti-PAR2 neutralizing, blocking or antagonising antibody need not be complete so long as it is detectable using an appropriate assay. In embodiments, the antibody or antigen-binding fragment thereof inhibits PAR2 activity at least 10%, 20%, 30%, 40%, 50%, 60%, 70%, 80%, 90% or 100% as compared to uninhibited active PAR2. Some examples of assays for detecting activity of a representative anti-PAR2 antibody or antigen-binding fragment are described in the exemplification section. The skilled worker is aware of additional anti-PAR2 antibody activity assays.

In embodiments, provided herein are antibodies or antigen-binding fragments that interfere with the interaction between PAR2 and a protease. In embodiments, the protease is trypsin. In embodiments, the protease is neutrophil elastase. In embodiments, the protease is neutrophil proteinase 3. In embodiments, the protease is mast cell tryptase. In embodiments, the protease is tissue factor/factor Vila/factor Xa. In embodiments, the protease is a kallikrein-related peptidase. In embodiments, the protease is membrane-tethered serine proteinase-1/matriptase 1. In embodiments, the protease is parasite cysteine proteinase.

In embodiments, the antibodies or antigen-binding fragments inhibit/reduce inflammation-induced pain.

Provided herein are PAR2-directed antibodies and antigen-binding fragments thereof that bind PAR2 molecules with high affinity at physiological, extracellular pH (i.e. pH 7.5).

The antibodies or antigen-binding fragments of the present disclosure may possess one or more of the aforementioned biological characteristics, or any combinations thereof. Other biological characteristics of the antibodies of the present disclosure will be evident to a person of ordinary skill in the art from a review of the present disclosure including the exemplification section provided herein.

In embodiments of the disclosure, the anti-PAR2 antibodies of the disclosure are human antibodies. The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs, and in embodiments, CDR3. However, the term “human antibody”, as used herein, is not intended to include antibodies in which CDR sequences derived from the germline of another mammalian species, such as a mouse, have been grafted onto human framework sequences.

The antibodies of the disclosure may, in embodiments, be recombinant human antibodies. The term “recombinant human antibody”, as used herein, is intended to include all human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes (see e.g., Taylor et al. 1992) or antibodies prepared, expressed, created or isolated by any other means that involves splicing of human immunoglobulin gene sequences to other DNA sequences. Such recombinant human antibodies have variable and constant regions derived from human germline immunoglobulin sequences. In embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In embodiments, the antibody or antigen-binding fragment thereof specifically binds to PAR2, and inhibits the binding of PAR2 activating peptide to PAR2 optionally wherein the antibody or fragment inhibits PAR2 activating peptide mediated accumulation of inositol monophosphate (IP) with an IC₅₀ value of less than about 50 pM, 100 pM, 200 pM, 400 pM, 800 pM, 1 nM, 5 nM, 10 nM, 20 nM, 40 nM, 80 nM, 100 nM, 200 nM or 300 nM, optionally wherein PAR2 peptide mediated accumulation of IP is determined using a PAR2 peptide stimulated IP signalling assay. In embodiments, the antibody or antigen-binding fragment thereof specifically binds to PAR2, and inhibits the binding of PAR2 activating peptide to PAR2, wherein the antibody or fragment inhibits PAR2 activating peptide mediated accumulation of inositol monophosphate (IP) with an IC₅₀ value of less than about 40 nM, 50 nM, 60 nM, 70 nM or 80 nM, optionally wherein PAR2 peptide mediated accumulation of IP is determined using a PAR2 peptide stimulated IP signalling assay. In embodiments, the antibody or antigen-binding fragment thereof specifically binds to PAR2, and inhibits the binding of PAR2 activating peptide to PAR2, wherein the antibody or fragment inhibits PAR2 activating peptide mediated accumulation of inositol monophosphate (IP) with an IC₅₀ of 40 nM or less, optionally wherein PAR2 peptide mediated accumulation of IP is determined using a PAR2 peptide stimulated IP signalling assay. In embodiments, the IP signalling assay is a Cisbio IP-One HTRF assay.

In embodiments, the antibody or antigen-binding fragment thereof inhibits trypsin mediated accumulation of IP with an IC₅₀ value of less than about 50 pM, 100 pM, 200 pM, 400 pM, 800 pM, 1 nM, 5 nM, 10 nM, 20 nM, 40 nM, 80 nM, 100 nM, 200 nM or 300 nM; optionally wherein trypsin mediated accumulation of IP is determined using a trypsin stimulated IP signalling assay. In embodiments, the antibody or antigen-binding fragment thereof inhibits trypsin mediated accumulation of IP with an IC₅₀ value of less than about 60 nM, 70 nM, 80 nM, 90 nM, 100 nM, 110 nM, 120 nM, 130 nM, 140 nM, 150 nM, 160 nM, 170 nM, 180 nM, 190 nM, 200 nM, 210 nM, 220 nM, 230 nM, 240 nM, 250 nM, 260 nM, 270 nM, 280 nM, 290 nM or 300 nM; optionally wherein trypsin mediated accumulation of IP is determined using a trypsin stimulated IP signalling assay. In embodiments, the antibody or antigen-binding fragment thereof inhibits trypsin mediated accumulation of IP with an IC₅₀ of 70 nM or less; optionally wherein trypsin mediated accumulation of IP is determined using a trypsin stimulated IP signalling assay.

In embodiments, the antibody or antigen-binding fragment thereof inhibits PAR2 activating peptide mediated accumulation of inositol monophosphate (IP) with an IC₅₀ value of less than about 50 PM, 100 pM, 200 pM, 400 pM, 800 pM, 1 nM, 5 nM, 10 nM, 20 nM, 40 nM, 80 nM, 100 nM, 200 nM or 300 nM, optionally wherein PAR2 peptide inhibition is determined using an HTRF assay.

In embodiments, the antibody or antigen-binding fragment thereof specifically binds to PAR2, wherein the antibody or fragment inhibits PAR2 activating peptide mediated calcium mobilisation, optionally with an IC₅₀ value of less than about 50 pM, 100 pM, 200 pM, 400 pM, 800 pM, 1 nM, 5 nM, 10 nM, 20 nM, 40 nM, 80 nM, 100 nM, 200 nM or 300 nM, and optionally wherein calcium mobilisation is determined using a PAR2 activating peptide stimulated calcium mobilisation assay. In embodiments, the antibody or antigen-binding fragment thereof specifically binds to PAR2, wherein the antibody or fragment inhibits PAR2 activating peptide mediated calcium mobilisation, optionally with an IC₅₀ value of less than about 50 pM, 100 pM, 200 pM, 400 pM, 800 pM, 1 nM, 5 nM, 10 nM, 20 nM, 40 nM, 80 nM or 160 nM, and optionally wherein calcium mobilisation is determined using a PAR2 activating peptide stimulated calcium mobilisation assay. In embodiments, the antibody or antigen-binding fragment thereof specifically binds to PAR2, wherein the antibody or fragment inhibits PAR2 activating peptide mediated calcium mobilisation, optionally with an IC₅₀ of 140 nM or less, and optionally wherein calcium mobilisation is determined using a PAR2 activating peptide stimulated calcium mobilisation assay.

In embodiments, the antibody or antigen-binding fragment thereof inhibits trypsin mediated calcium mobilisation, optionally with an IC₅₀ of less than about 50 pM, 100 pM, 200 pM, 400 pM, 800 pM, 1 nM, 5 nM, 10 nM, 20 nM, 40 nM, 80 nM, 100 nM, 200 nM or 300 nM, and optionally wherein calcium mobilisation is determined using a PAR2 activating peptide stimulated calcium mobilisation assay. In embodiments, the antibody or antigen-binding fragment thereof inhibits trypsin mediated calcium mobilisation, optionally with an IC₅₀ of less than about 50 pM, 100 pM, 200 pM, 400 pM, 800 pM, 1 nM, 5 nM, 10 nM, 20 nM, 40 nM, 80 nM, 100 nM, 200 nM or 250 nM, and optionally wherein calcium mobilisation is determined using a PAR2 activating peptide stimulated calcium mobilisation assay. In embodiments, the antibody or antigen-binding fragment thereof inhibits trypsin mediated calcium mobilisation, optionally with an IC₅₀ 200 nM or less, and optionally wherein calcium mobilisation is determined using a PAR2 activating peptide stimulated calcium mobilisation assay

In embodiments, the antibody or antigen-binding fragment thereof binds to cynomolgus PAR2 with an EC₅₀ of less than about 50 pM, 100 pM, 200 pM, 400 pM, 800 pM, 1 nM, 5 nM, 10 nM, 20 nM, 40 nM, 80 nM, 100 nM, 200 nM or 300 nM, optionally wherein cynomolgus PAR2 binding is determined using flow cytometry. In embodiments, the antibody or antigen-binding fragment thereof binds to cynomolgus PAR2 with an EC₅₀ of less than about 400 pM, 800 pM, 1 nM, 5 nM or 10 nM, optionally wherein cynomolgus PAR2 binding is determined using flow cytometry.

In embodiments, the antibody or antigen-binding fragment thereof binds to human PAR2 with a K_{D} of less than about 50 pM, 100 pM, 200 pM, 400 pM, 800 pM, 1 nM, 5 nM, 10 nM, 20 nM, 40 nM, 80 nM, 100 nM, 200 nM or 300 nM, optionally wherein binding affinity is determined using surface plasmon resonance (SPR) or KinExA. In embodiments, the antibody or antigen-binding fragment thereof binds to human PAR2 with a K_{D} of less than about 100 pM, 200 pM, 400 pM, 800 pM, 1 nM, 5 nM, 10 nM or 20 nM, optionally wherein binding affinity is determined using surface plasmon resonance (SPR) or KinExA. In embodiments, the antibody or antigen-binding fragment thereof binds to human PAR2 with a K_{D} of 10 nM or less, optionally wherein binding affinity is determined using surface plasmon resonance (SPR) or KinExA.

### FIGURES

FIG. 1: Examples of specific PAR2 nanodisc (FL-StaR ND) versus empty nanodisc (empty ND) binding by positive control mAbs ‘Benchmark 1’ and a commercially available PAR2 mAb ‘R&D anti-huPAR2 mAb (MAB3949)’ and dose dependent binding observed for these and the three PAR2 phage-derived antibody clones shown. Antibodies were directly coated onto plates at 5 μg/mL. No significant background binding observed with the negative control, MOR03207, an anti-lysozyme antibody. Data are shown as signal divided by background (S/BG).

FIG. 2: Exemplification of degrees of competition (complete, partial and no competition) with the commercially available positive control mAb ‘MAB3949’ (R&D anti-huPAR2) on a panel of PAR2 mAbs (derived from the Ylanthia phage antibody library) using full length PAR2 StaR nanodiscs and therefore an indication of diversity. 50 nM candidate mAb was titrated against a dose-response of R&D anti-huPAR2 (MAB3949) in competition for full length StaR nanodisc. Both complete competition and partial competition demonstrate a dose dependent binding effect. No dose dependent binding effect or competition with MAB3949 is observed for the reference Abs ‘Benchmark 1’ (an N terminal binder) or ‘MOR03207’ (an anti-lysozyme antibody used as a negative control). Data are shown as signal divided by background (S/BG).

FIG. 3: Dose dependent binding as measured by FACS of: A) Benchmark 1 mAb and MAB3949 to cell surface-expressed PAR2 on CHO_V5HIS-huPAR2 cells vs CHO parental cells to demonstrate validation of assay set-up. BG=background; B) clone Y022065 to recombinantly expressed PAR2 on CHO_V5HIS-huPAR2 cells vs CHO parental cells; C) clone Y022065 to A549 cells that endogenously express human PAR2 and mouse PAR2-expressing CHO cells. BG=background. Data are shown as relative luminescence units (RLU).

FIG. 4: Understanding the ability of antibody clones to bind full length huPAR2 (FL-N, second row) and N-terminally truncated huPAR2 (truncate, first row) by flow cytometry. The resulting data demonstrates the ability to identify not only antibody clones that preferentially bind to the N terminus (full length receptor, e.g. Y022066), but also other antibody clones (e.g. Y022065, Y022071) that bind to the truncated AND full length receptor, where the epitope also comprises part of the extracellular domain, and clones that bind the truncated but not full length receptor (e.g. Y022075).

FIG. 5: Multiplex profiling using IntelliCyt. The graphs show binding of purified IgG1f_AEASS on human PAR2-expressing cells. Data are exemplary for clone ‘Y022065’ (functional candidate). Top) HEK Flp-In TRex 293_huPAR2 expression-induced, HEK Flp-In TRex 293_huPAR2 non-induced, Flp-In CHO_V5His_huPAR2, parental Flp-In CHO cells and Flp-In CHO_huPAR1. Bottom) HEK293F infected BacMam WT FL-huPAR2 and HEK293F non-infected. Data are shown as signal divided by background (S/BG).

FIG. 6: Functional characterization of Y021171, Y022063 and Y022054 & Y022065 hIgG1f_AEASS in the peptide inhibition assay using the Cisbio® IP-One Gαq assay at two concentrations in replicate. Statistically significant inhibition of Activating Peptide induced agonism at human PAR2 was observed for four clones against the response of 6.28 nM agonist alone ranging from 17-43% at the highest concentration tested. Y022065 identified as the most active candidate. Data are shown as mean response with standard deviation from 2 independent experiments run in singlicate.

FIG. 7: Koff-ranking ELISAs using nanodisc-embedded human PAR2 StaR® and purified soluble StaR® protein, where parental candidates represent the lineages of the 22 matured IgG candidates identified as functionally dual-active. Data are shown as signal divided by background (S/BG).

FIG. 8: Binding specificity of affinity matured clones shown by parental lineage on FlpIn CHO-V5His-huPar2 versus FlpIn CHO parental cells.

FIG. 9: Affinity matured clones, formatted as Fab fragments, bind to human PAR2 expressing CHO cells as assessed by flow cytometry. Data are shown as signal divided by background (S/BG).

FIG. 10: Affinity matured clones, formatted as IgGs or fAbs, bind to cynomolgus (IgGs)- and human-PAR2 (fAbs) BacMam-infected HEK293 cells as assessed by flow cytometry. Data are shown as signal divided by background (S/BG).

FIG. 11: Confirmation of binding to human and cynomolgus PAR2 expressing cells for the affinity matured lead mAb panel as Fab fragments and IgG in comparison to benchmark mAbs. Antibodies were incubated overnight with human PAR-2 and cyno PAR-2 over-expressing cells at ˜5° C. with sodium azide. All antibodies show clear binding to cyno PAR-2 and human PAR-2 both as Fab fragments and as intact IgG. Data are shown as signal divided by background (S/BG).

FIG. 12: Functional characterization of lead optimized clones in inhibition assay against PAR2 Activating Peptide. The IP-One assay is used to measure accumulation of IP as a function of Gαq activation and human PAR2 activity in vitro. Each value represents % inhibition normalized against 10 μM MAB3949 at highest concentration tested in replicate for individual lead clones. Lead clones have been aligned with parental IgG. Inhibition of Activating Peptide was observed for lead clones matured from all parental IgGs. Percentage inhibition ranges from 0 through to ˜250% across the clones tested relative to the R&D Systems anti-human PAR2 MAB3949, suggesting that at comparable concentrations, a high number of lead clones show greater inhibition of Activating Peptide agonism of human PAR2 when compared to MAB3949. Lead clones derived from parent IgGs Y021171 and Y022065 by affinity maturation exhibit the largest proportion of actives.

FIG. 13: Functional characterization of lead optimized clones in inhibition assay against Bovine Trypsin. The IP-One assay is used to measure accumulation of IP as a function of Gαq activation and human PAR2 activity in vitro. Each value represents % inhibition normalized against 1 μM Benchmark 1 at highest concentration tested in replicate for individual lead clones. Lead clones have been aligned with parental IgG. Inhibition of Bovine Trypsin was observed for lead clones matured from all parental IgGs. Percentage inhibition ranges from 0 through to ˜130% across the clones tested relative to Benchmark 1. Lead clones identified from maturation of IgG Y022065 demonstrate the largest proportion of actives against Bovine Trypsin challenge. Two representatives derived from Y021171 demonstrate the highest % inhibition.

FIG. 14: Graphical representation of dose response inhibitor curves of parental clone (Y022065) and affinity matured lead representatives (Y022870, Y022877, Y022883) compared with Benchmark 1 and Benchmark 2 in calcium mobilisation assay measured in response to HT-29 challenge with Bovine Trypsin to activate endogenous PAR2. Data are shown as mean response with standard deviation from 3 independent experiments run as duplicate wells.

FIG. 15: SPR evaluation by Biacore to determine effect of pH on binding of Y022883 to PAR2.

FIG. 16: Affinity determination by KinExA of lead candidates Y022870 and Y022883 on HEK-293F-human PAR2 expressing cells.

FIG. 17: Epitope binning matrix of affinity matured clones by parental family derivation. Anti-huPAR2 is MAB3949.

FIG. 18: Back view of PAR2 (ECL3, Segment1 and Helix0/1). Regions of PAR2 that interact with Y022883, as derived by HDX, are shown as hatched areas.

FIG. 19: Top view of PAR2 (ECL3, Segment1 and Helix0/1). Regions of PAR2 that interact with Y022883, as derived by HDX, are shown as hatched areas.

FIG. 20: Side view of PAR2 (ECL3, Segment1 and Helix0/1). Regions of PAR2 that interact with Y022883, as derived by HDX, are shown as hatched areas.

FIG. 21: Expression profiles of WT and mutated (S60W, D62F, G318F, D62G & E63G) human PAR2 detected using flow cytometry of BacMam-infected HEK293F cells stained with Benchmark 1 (an N-terminal binder) or Y022883. 2^{nd} only: secondary FACS antibody only; n.i.: not infected.

FIG. 22: Rat serum mAb concentrations versus time following a single intravenous dose of 10 mg/kg Y022883. Data are shown as mean (open circles) with 95% confidence intervals (solid bars) from 3 male, adult animals; individual animal data are also shown (filled circles); detectable mAb concentrations at or below the BLOQ (5 ng/ml) are reported as BLOQ/2 (dashed line).

FIG. 23: Cynomolgus monkey serum mAb concentrations versus time following a single intravenous dose of 10, 3 or 1 mg/kg Y022883. Data are shown as mean (10 mg/kg open circles, 3 mg/kg solid squares, 1 mg/kg solid triangles) with 95% confidence intervals (solid bars) from 3 male, adult animals; individual animal data are also shown (10 mg/kg solid circles, 3 mg/kg open squares, 1 mg/kg open triangles); detectable mAb concentrations at or below the BLOQ (5 ng/ml) are reported as BLOQ/2 (dashed line).

FIG. 24: Cynomolgus monkey ex vivo pharmacodynamics following a single 10 mg/kg dose of Y022883. Data are shown as the percentage of pre-dose stimulant-induced (PAR2-AP as circles, trypsin as squares, LPS as triangles) gene signature.

FIG. 25: Cynomolgus monkey ex vivo pharmacodynamics following a single 3 mg/kg dose of Y022883. Data are shown as the percentage of pre-dose stimulant-induced (PAR2-AP as circles, trypsin as squares, LPS as triangles) gene signature.

FIG. 26: Cynomolgus monkey ex vivo pharmacodynamics following a single 1 mg/kg dose of Y022883. Data are shown as the percentage of pre-dose stimulant-induced (PAR2-AP as circles, trypsin as squares, LPS filled triangles) gene signature.

FIG. 27: Inhibition of Trypsin induced phosphorylation of p38-MAPK and pERK in T84 cells by Y022883 (SH-C) and Benchmark II (SH-D). Data show levels relative to treatment with vehicle and are normalised to total ERK or p38-MAPK.

FIG. 28: Inhibition of PAR2-AP induced phosphorylation of p38-MAPK and pERK in T84 cells by Y022883 (SH-C) and Benchmark II (SH-D). Data show levels relative to treatment with vehicle and are normalised to total ERK or p38-MAPK.

FIG. 29: Inhibition of Trypsin and PAR2-AP induced phosphorylation of pERK (Left) and p38-MAPK (Right) in T84 cells by Y022883 (SH-C) and Benchmark II (SH-D). Data show levels as a percentage of the difference between the vehicle and the positive control.

### EXAMPLES

Certain aspects and embodiments of the invention will now be illustrated by way of example and with reference to the figures described above.

### Example 1-Generation of Full-Length and N-Terminally Truncated Human PAR2 Constructs

### Designing PAR2 Constructs

The gene of the human Protease-Activated Receptor 2 (PAR2) was edited using standard site-directed mutagenesis strategies, based on Hutchison et al., 1978, to stabilize the receptor in the antagonist conformation by the introduction of nine point mutations (Cheng et al 2017). The stabilized receptor was also either genetically truncated at the N-terminus by 54 amino acids (first residue in encoded construct is V55) or kept full length (FL-N) using Polymerase Chain Reaction (PCR, based on Saiki et al., 1985), widely known in the Art. All four PAR2 proteins are also genetically truncated at the C-terminus by 20 amino acids (last residue K377) as part of the stabilized receptor process.

The following constructs were generated:
PAR2-1: P151: 55-377, glycosylated, pFastBacHisStrepII, truncated
PAR2-2: P166: 1-377, de-glycosylated: N30Q, N222Q, pFastBacHis StrepII, full length
PAR2-3: P157: 1-377, glycosylated, pBacMamHisStrepII, full length
PAR2-4: P155: 55-377, glycosylated, pBacMamHisStrepII, truncated

The rationale for generating an N terminally truncated PAR2 receptor that was sufficiently stable for use as antigen was to enable the discovery and identification of antibodies that bound to other regions of the receptor other than just the N terminus, thereby generating a novel antibody.

The potential glycosylation sites N30 and N222 were substituted by site-directed mutagenesis to glutamine residues in the FL-N protein PAR2-2.

StrepII and deca-histidine tags were added by PCR and endonuclease cloning strategies, known to the skilled worker, to the C-terminus to allow affinity purification and specific selection in ELISA assays. PAR2-1 and PAR2-2 genes were cloned into a pFastBac1 vector (Thermofisher, #10360014). The multiple cloning site of the vector was modified to allow for insertion of genes using NheI as restriction endonuclease. The pFastBac1 vector is part of the commercial Bac-to-Bac expression system (Smith et al., 1983, Thermofisher, #10359016) for insect cells, which is known and widely used in the art. We followed the manufacturer's instructions. The Bac-to-Bac expression system was used for PAR2-1 and -2 proteins. The pFastBac1 vector was modified to generate the pBacMam vector by introducing a human cytomegalovirus promotor 3′ following the polyhedron promotor, to allow for protein expression of the protein PAR2-3 and PAR2-4 in mammalian cells. The instructions of the Bac-to-Bac expression system were also applied to the pBacMam virus generation.

### Expression

Proteins PAR2-3 and PAR2-4 were expressed in Human Embryonic Kidney 293 F Cells (Gibco 293F cells, Thermofisher Scientific, #11625019) in Lonza Pro293s CDM media (#BE02-025Q) with 10% fetal bovine serum albumin (FBS, Sigma-Aldrich, #F9665) and supplemented with 5 mM sodium butyrate (Sigma-Aldrich, #303410) by virus infection at a cell density of 2.5×10\hat{ }6/ml with 2.7% virus for 48 h. Cells were harvested and processed as described below for Sf9-cell expression. Mammalian cell lines were used for expression of constructs in order to provide options to utilize antigen that contained mammalian-like glycosylation patterns.

Proteins PAR2-1 and -2 were expressed in Spodoptera frugiperda Sf9 cells (Thermofisher, #89070101) in Expression Systems ES921 media (#96-001-01) with 10% FBS (Sigma-Aldrich, #F9665). Cells were infected at a multiplicity of infection (MOI) of 2 at a cell density of 3.5×10\hat{ }6/ml. Cells were harvested by centrifugation after 48 h, washed with 50 mM HEPES PH 7.5, 250 mM NaCl (PAR2 buffer) supplemented with Roche EDTA-free Complete protease inhibitors (PI, Sigma-Aldrich, #5056489001) and stored at −80° C. To prepare cellular membranes, cells were re-suspended in PAR2 buffer, supplemented with PI-tablets (PAR2/PI), and then homogenized by a single pass through a Microfluidics Microfluidizer Processor. The membranes were collected by ultra-centrifugation for 1 hr at 135,000 g in a Beckman 45 Ti rotor, homogenized into PAR2/PI buffer and stored at −80° C. All subsequent purification steps were performed at 4° C.

### Purification

Thawed membranes were solubilized by the addition of 1% (w/v) LMNG (Anatrace, #NG310)/0.1% (w/v) cholesteryl hemisuccinate (CHS, Anatrace, #210) mixture for 1 hour. Insoluble material was removed by ultra-centrifugation for 30 minutes at 205,000 g in a Beckman 45 Ti rotor. Protein was batch bound for 2 hours to 8 ml NiNTA Superflow resin (Qiagen, #30430) in the presence of 8 mM imidazole. The resin was packed into a XK 16/20 column (GE Healthcare, #GE28-9889-37) and washed with 15 column volumes (CVs) of high-salt buffer A1 (50 mM HEPES pH 7.5, 500 mM NaCl, 0.02% LMNG, 0.002% CHS, 75 mM imidazole) and 3 CVs of buffer A2 (50 mM HEPES pH 7.5, 250 mM NaCl, 0.02% LMNG, 0.002% CHS, 75 mM imidazole). Protein was eluted with buffer A2 supplemented with 300 mM imidazole and concentrated to 0.5 ml using an Amicon Ultra-15 recycled cellulose concentrator with 100 kDa molecular weight cut-off (Millipore, #UFC900308). Aggregated material was removed by ultracentrifugation at 220,000 g for 10 min in a Beckman Coulter benchtop centrifuge using the TLA-100.2 rotor. The sample was then subjected to size exclusion chromatography in 50 mM HEPES pH 7.5, 150 mM NaCl, 0.02% LMNG, 0.002% CHS, on a Superdex 200 10/300 GL column (GE Healthcare, #17-5175-01). Fractions corresponding to the monomeric species were pooled and concentrated up to 6-8 mg/ml using a Vivaspin 500 concentrator with a 100 kDa molecular weight cut off (Vivaproducts, #VS0242). Sample purity and monodispersity were analyzed by SDS-PAGE and analytical gel filtration. Protein concentration was determined using the molecular weight, extinction coefficient and protein absorbance at 280 nm with the Nanodrop Spectrophotometer (Thermofisher Scientific). The protein was checked by mass spectrometry to confirm the protein of interest.

### Preparation of Nanodisc-Embedded Human PAR2 StaR Protein

The preparation of nanodiscs is known in the art and based on Banerjee et al., 2008, using zebrafish apolipoprotein-1 (ZAP1) as the scaffold protein with a N-terminal hexa-histidine tag. Proteins PAR2-1 and -2 were re-constituted at 100 to 200 μM into nanodiscs and used as antigens for Fab-selection. To reconstitute nanodiscs, firstly the lipids below were resuspended in an aqueous 200 mM sodium deoxycholate (Sigma-Aldrich, #6750) solution: POPC (1-palmitoyl-2-oleoyl-sn-glycero-3-phosphocholine, Anatrace, #P616), POPG (1-Hexadecanoyl-2-(9Z-Octadecenoyl)-sn-Glycero-3-Phosphoglycerol, Anatrace, #P516), and cholesterol (Anatrace, #CH200) in a ratio of 3:2:0.5. The final molar ratios were 1:72:48:12:264 for PAR2:POPC:POPG:cholesterol:sodium-deoxycholate, respectively. The final sodium deoxycholate concentration was kept above 15 mM and adjusted with 10 mM HEPES 7.4, 150 mM NaCl buffer.

After a 30 minute incubation on ice, zebrafish apolipoprotein 1 (ZAP1) was added in a molar ratio of 1:2 PAR2: ZAP1. The mix was incubated for 1 hour on ice. Then, the detergent was removed by agitating the sample over night with Bio-beads SM-2 (Bio-Rad, #1528920) in a 1:1 ratio of protein solution to dry Bio-Bead weight (e.g. 900 μl protein to 900 μg Bio-Beads). The nanodiscs were recovered as supernatant and the beads were washed with 50 mM HEPES PH 7.5, 150 mM NaCl for two volumes worth of weight of beads (e.g. 1.8 mL for 900 μg beads). The protein solution was concentrated below 1 mL, centrifuged for 15 min in a table top centrifuge at 13,500 g and the supernatant was subjected to size exclusion chromatography on a Superdex 200 10/300 GL column (GE Healthcare), as used above, in 50 mM HEPES pH 7.5, 150 mM NaCl. Fractions representing the nanodisc peak were pooled and concentrated to 1-2 mg/ml in an Amicon Ultra-4 recycled cellulose concentrator (100 kDa molecular weight cut-off, Millipore, #UFC810096) and frozen in aliquots at −80° C.

### Example 2—Screening

Antibodies or antigen-binding fragments of the disclosure were identified from phage display libraries. A variety of methods are known in the art for generating phage display libraries and screening such libraries for antibodies possessing the desired binding characteristics. The phage display library used to identify the antibodies provided herein is the Ylanthia® phagemid based on the Ylanthia (Tiller et al. 2013) concept and employs CysDisplay™ technology to display Fab on the phage surface (Lohning et al. 2000).

Ten panning strategies, (including solid phase panning, capture panning and whole cell panning using techniques known in the art), were designed with an emphasis on solid phase pannings using full length or N-terminally truncated nanodisc-embedded human PAR2 StaR protein. Various antigen presentation techniques were implemented to guarantee the integrity and stability of the nanodisc-embedded PAR2 StaR and to lower the risk of enrichment of candidates specific against other unwanted epitopes during the panning process. Nanodisc-embedded human PAR2 StaR variants were used as panning antigen (i.e., a detergent free state). For most panning strategies, nanodisc-embedded human PAR2 StaR protein was presented via tag-specific capture antibodies. Alternation of the capture mode was implemented to lower the risk of enrichment of candidates specific for the capture antibody Benchmark 1 (Giblin et al 2011), anti-Histidine-Ab IgG1 (StrepMAB-Immo) or reagent (NINTA). For some strategies, differential whole cell pannings were conducted on human PAR2-expressing Flp-In CHO and BacMam-infected HEK293F cells. Alternation of panning on StaR followed by cell panning was employed to lower the risk of enrichment of candidates specific for other cellular targets (off-target binding). To amplify phage-antibodies competing with functionally active ligands of human PAR2, pannings were conducted using nanodisc embedded human PAR2 StaR variants PAR2-2 P166: 1-377, de-glycosylated: N30Q, N222Q, pFastBacHis StrepII or hPAR2 (55-377_10xH_Sx2 (truncated) PAR2-1: P151: 55-377, glycosylated, pFastBacHisStrepII) saturated with the commercially available small molecule ligands, AZ8838 and AZ3451 (Cheng et al 2017). These pannings lead to the identification of a large number of Ylanthia® candidates with mostly good specificity for the human PAR2 receptor expressed in a number of different cellular backgrounds. For a more stringent deselection of non-specifically bound phage, a pre-adsorption on irrelevant antigen components was performed prior to each round of solid phase pannings. Irrelevant antigen components included Ni-NTA, empty nanodisc as well as capture antibodies anti-StrepII mAb (StrepMAB-Immo) and anti-poly 6× Histidine-Ab IgG1 (R&D MAB050) or a nanodisc-embedded alternative GPCR (used exclusively in maturation pannings).

### Phage Preparation

Production of Fab-presenting phage particles: New phage particles presenting Fab fragments on their surface were produced for each selection round. Thereby an E. coli TG1 culture was infected with phage derived from the previous selection round. Upon centrifugation, the bacterial pellets were re-suspended in fresh medium and plated on agar plates. After outgrowth, colonies were scraped off the plates and were used for phage rescue, polyclonal amplification of selected clones and phage production. With purified phage, the next panning round was started. Upon the last panning round single clones were picked from agar plates into the wells of a sterile microtiter plate pre-filled with medium. Upon outgrowth, medium containing glycerol was added into each well of the master plates; plates were sealed with aluminum foil and stored at −80° C.

### Example 3—Identification of Hits by ELISA and Whole Cell Binding by FACS and IntelliCyt Screening ELISA

ELISA techniques have been used for both screening of single Fab clones identified from panning outputs on target antigens as well as for characterization of purified antibodies. Optimal antigen and antibody concentrations as well as blocking conditions were determined according to state of the art methods.

### Direct Coating of Antigen

Antigens were immobilized on microtiter plates. Plates were blocked and incubated with antibodies such as Fab containing crude E. coli lysates or purified Fab or IgG samples. Bound antibodies were detected using alkaline-phosphatase (AP) coupled secondary antibodies in combination with ‘AttoPhos’ fluorescence substrate. Multiple washing steps were performed in between individual assay steps.

### Antigen Capture

In other ELISA settings, antigens were captured to plates via a tag-specific antibody coated on microtiter plates (e.g. anti-StrepII, anti-His or benchmark 1 reference mAb). Bound antibodies were detected using respective alkaline-phosphatase (AP) coupled secondary antibodies in combination with ‘AttoPhos’ fluorescence substrate. Multiple washing steps were performed in between individual assay steps.

### Fab Expression Check by Anti-Fd ELISA

For verification of Fab expression in crude bacterial lysates, plates were coated with Fd-fragment specific antibodies. Bound Fabs were detected using respective alkaline-phosphatase (AP) coupled anti-Fab specific antibody in combination with ‘AttoPhos’ fluorescence substrate. Multiple washing steps were performed in between individual assay steps.

### Koff Ranking ELISA

For a Koff estimate, the ELISA plate was subjected to additional frequent and stringent washing upon the initial readout and a second detection was performed to identify candidates with a slow kd. Washing was performed using the following conditions: washing with 10 minute incubation 5 times, followed by overnight incubation, and then washing 5 times with 10 minute incubation, followed by 1h incubation, then washing 5 times with 5 min incubation, before detection. Bound antibodies were detected using respective alkaline-phosphatase (AP) coupled secondary antibodies in combination with ‘AttoPhos’ fluorescence substrate.

### Implementation of ELISA Methods

ELISA techniques were used for both screening of single Fab clones identified from panning outputs on target antigens as well as for characterization of purified antibodies. Optimal antigen and antibody concentrations as well as blocking conditions were evaluated and settings adjusted.

Following panning selections, 368 clones from each third panning round output, were processed in primary screenings as bacterial lysates. Panning outputs from solid phase pannings were screened by FACS on StaR-coated magnetic beads or in ELISA using tagged-StaR variants or nanodisc-embedded StaRs. Screenings were mainly performed on the PAR2-1 P151 variant (55-377, glycosylated, pFastBacHisStrepII). Counter screenings included primarily empty nanodiscs and also an anti-Fd mAb and mouse gamma globulin.

Secondary screenings were performed primarily in ELISA. The antigen panel included PAR2-1: P151: 55-377, glycosylated, pFastBacHisStrepII as well as nanodisc-embedded PAR2-2: P166: 1-377, de-glycosylated: N30Q, N222Q, pFastBacHis StrepII saturated with the ligands AZ8838 and AZ3451. Similarly, counter screenings were extended to the capture antibody StrepMAB-Immo and bRIL-His-StrepII. Despite low binding signals on cells, all clones of interest displayed a good correlation between cell binding and binding profiles gained on recombinant nanodisc-embedded StaR material.

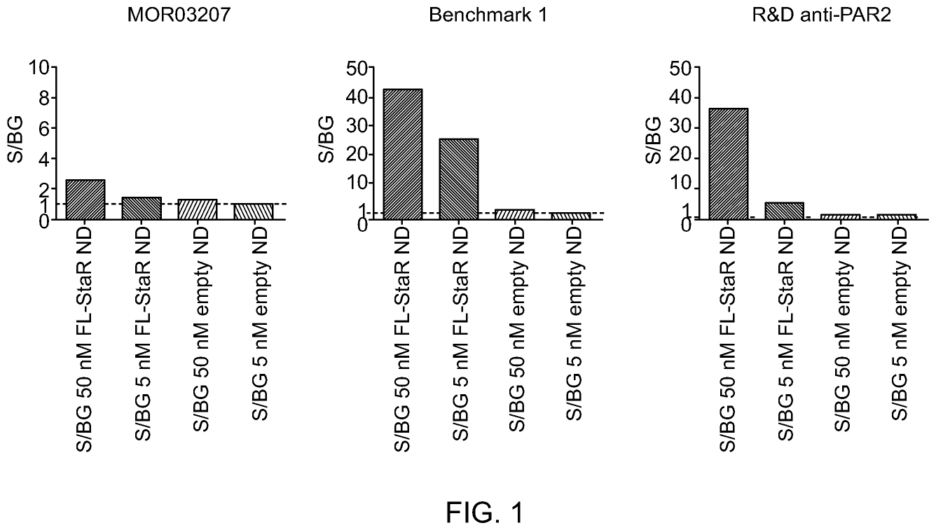

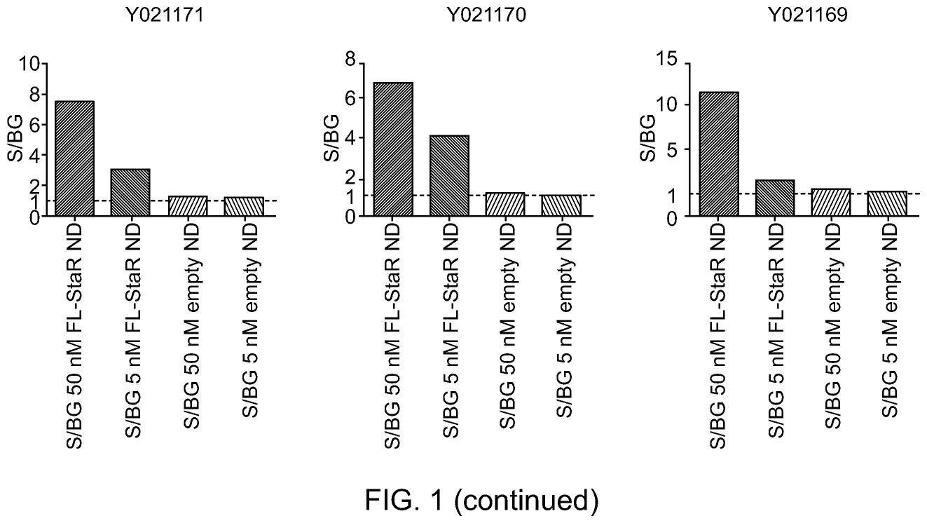

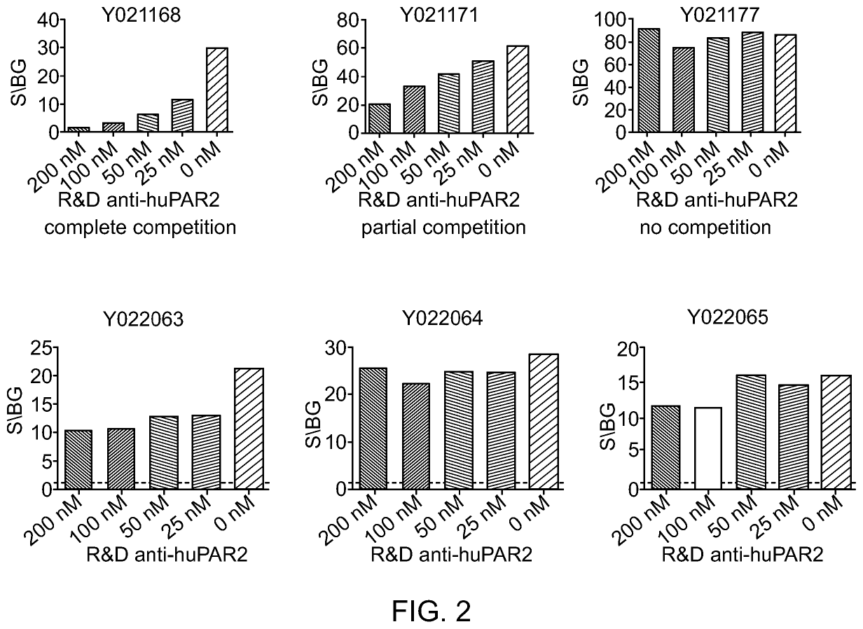

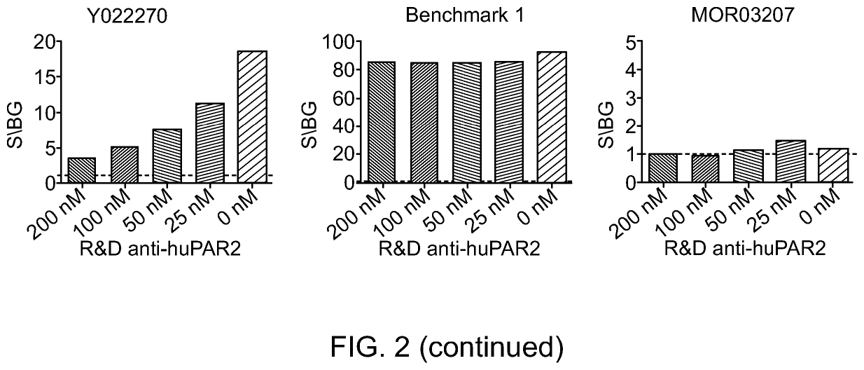

These were selected for sequencing and IgG conversion into the silent h_IgG1f_AEASS format (IgG1 L234A L235E G237A A330S P331S. This format has been clinically validated in Novo Nordisk's second generation anti-C5aR antibody NNC0215-0384 (Wagner et al., 2014)), expressed and purified as advanced micro scale and/or exploratory scale for characterization. Prior to functional characterization, the IgGs were re-screened by ELISA (FIGS. 1 and 2) and for cell binding by IntelliCyt to reconfirm binding specificity.

### FACS and IntelliCyt

### Cell Binding by Flow Cytometry and IntelliCyt to Screen for Positive Identification of PAR2 Binders

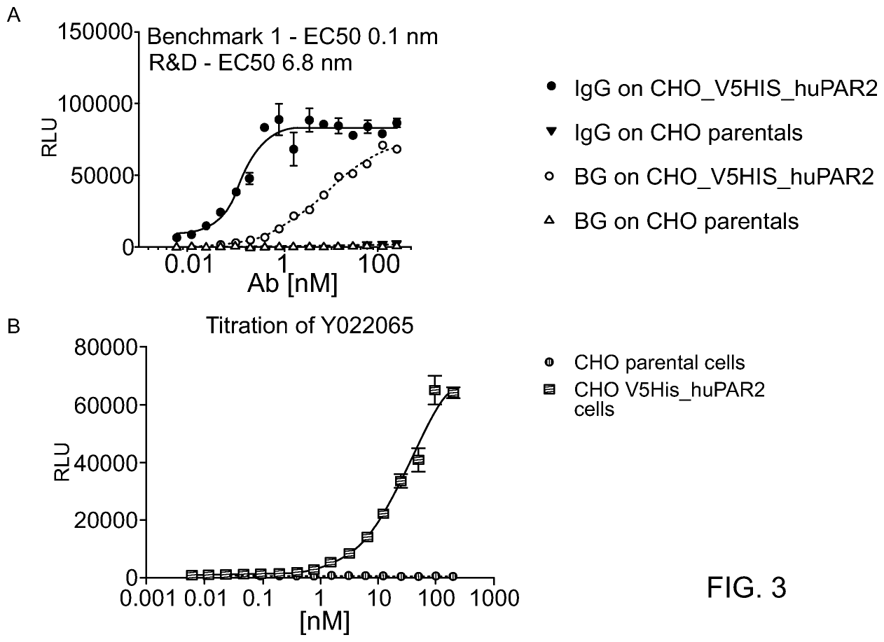

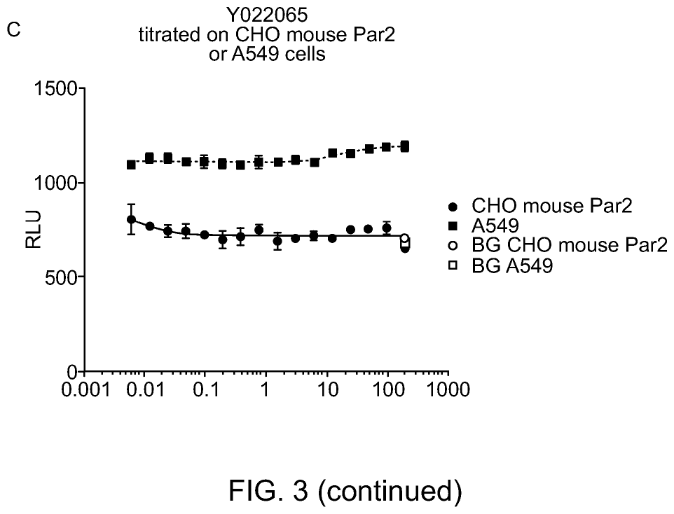

Binding events to cell surface expressed antigen were identified by flow cytometry using either crude E. coli lysates from the panning output or purified antibodies. High throughput primary screens of panning outputs from differential whole cell pannings (DWCP) were mainly performed on human wild type PAR2-expressing Flp-In CHO cells (Flp-In CHO_huPAR2), GNTI BacMam wild type PAR2 cells and GNTI BacMam truncated PAR2 StaR versus parental Flp-In CHO cells (PAR2 negative) or non-infected HEK293GNTI-cells. Candidates showing an elevated background binding to parental Flp-In CHO cells were ascribed as either non-specific or off-target binders. All steps were performed in FACS buffer including FCS and sodium azide to prevent potential receptor internalization. Cell suspensions were transferred into microtiter plates and antibody samples were added followed by subsequent incubation of plates at 4° C. Sample volume and cell-number were adjusted to the plate type used. Following incubation, cells were spun down and washed with FACS buffer. Fluorophore-conjugated secondary reagents were used for detection of bound antibodies. Plates were measured using the BD FACS Array or IntelliCyt HTFC/iQue System and data was analysed using FlowJo, ForeCyt or proprietary software tools (FIG. 3).

### Evaluation of the Nature of Whole Cell Binding/Specificity (FACS, IntelliCyt)

### Binding Specificity as Evaluated by Flow Cytometry on Human PAR2 Expressing Sf9 Cells

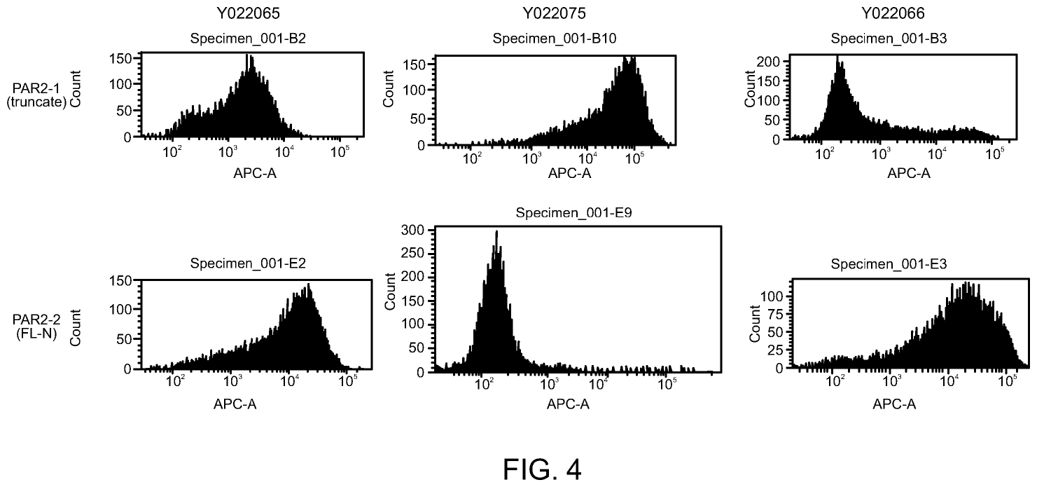

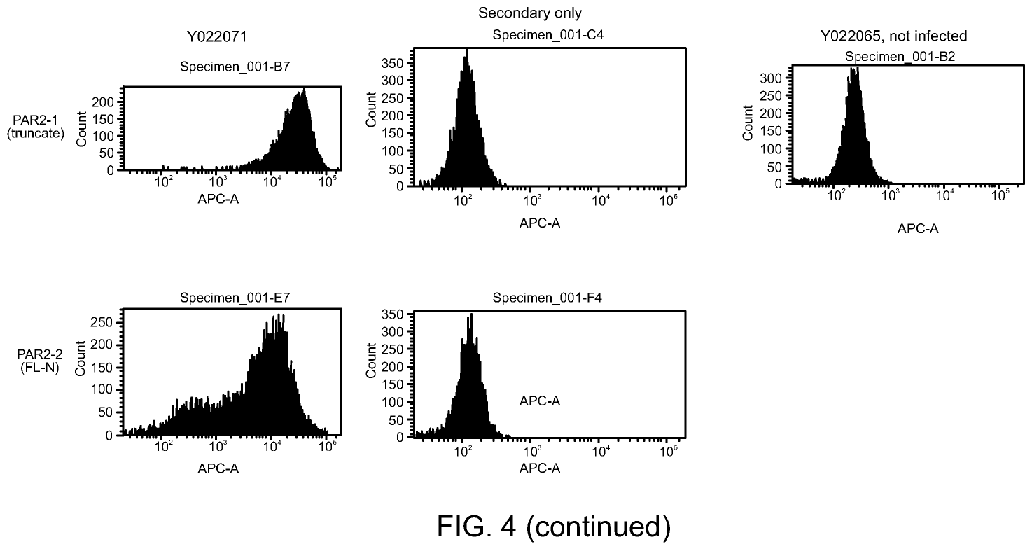

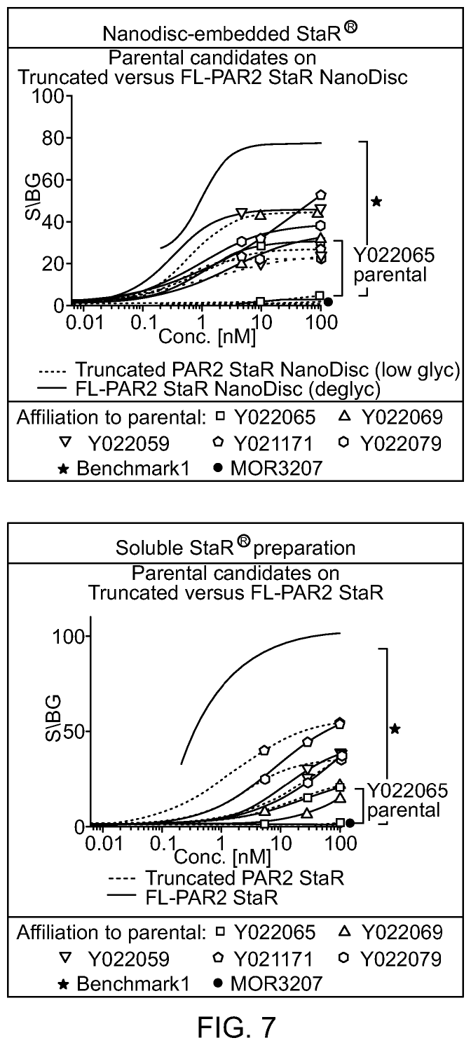

FACS-based analysis of initial PAR2 candidates was performed using PAR2-expressing Sf9 insect cells. In order to understand whether the antibody binds preferentially to the N-terminus or the rest of the extracellularly exposed part of the receptor, cells were either infected with full-length N-terminus expressing PAR-2 StaR (PAR2-3, 1—377, here depicted as FL-N StaR) or N-terminally truncated receptor (PAR2-4, 55—377, here depicted as NA53 StaR, or in FIGS. 4 and 7 as “truncated”).

200 μL Sf9-cells at 4×10\hat{ }6 cells/mL in FACS-buffer (PBS (Sigma, #F9665), 1% BSA (Sigma, #A9647) and Roche Complete protease inhibitors (#11836145001)) were incubated with the antibody of interest at 20 nM for 1 hour at room temperature. Cells were pelleted and washed three times with 200 μL FACS buffer. Successively, the cells were incubated with 200 μl secondary allophycocyanin (APC) conjugated anti-human IgG at 20 nM for 1 hour and washed again three times with 200 μL FACS buffer.

Read-outs were monitored using BD FACS Canto II FACS instrumentation.

A range of binders were generated with different binding profiles as determined by flow cytometry, where Y022075 binds preferentially to the truncated receptor, and Y022065 binds to both the truncated receptor and to full length PAR2. In contrast, antibody clone Y22066 binds almost exclusively to the FL-N construct. These observations reflect a diversity of epitopes (FIG. 4).

### Binding Specificity as Determined by IntelliCyt

Binding specificity was confirmed for purified IgGs and was performed as 16-point titration in duplicates on human PAR2-expressing cells as n=2 experiment. The following cell lines were included in the analysis:
Flp-In CHO_V5His_huPAR2 versus parental Flp-In CHO cells
Flp-In CHO_huPAR1 versus parental Flp-In CHO cells
HEK Flp-In TRex 293_huPAR2 expression-induced versus non-induced cells
HEK293F infected BacMam WT FL-huPAR2 versus non-infected cells
HEK293F infected BacMam WT FL-marmoset PAR2 versus non-infected cells

All clones of interest arose from a PAR2 nanodisc panning strategy, which enabled an orientated presentation by virtue of antibody capture via tagged receptor and/or nanodisc scaffold, and displayed selective binding to PAR2-expressing cells in different cellular backgrounds. PAR2 StaR nanodisc preparations presented high quality PAR2 receptors, in a detergent-free environment, comparable to the native receptor expressed on cells and led to the identification of highly promising, functionally active candidates. In addition, panning stringency is easily controlled using recombinant material, such a StaR nanodisc.

### Multiplexing by IntelliCyt

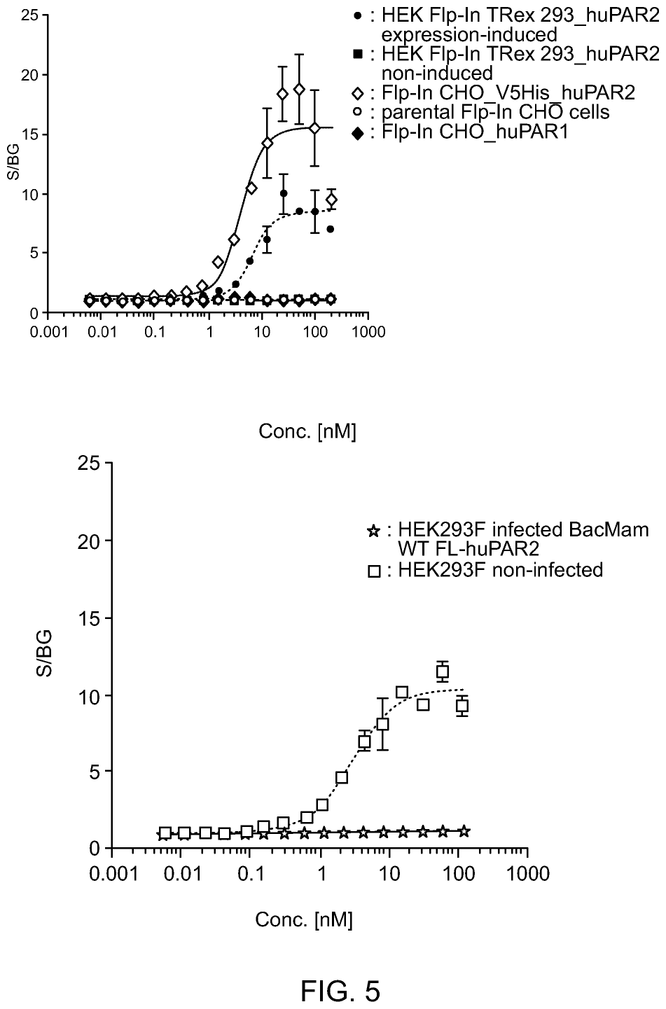

To evaluate species cross-reactivity and/or unwanted binding to counter-targets simultaneously, screening in 384-well plate format was performed using the HTFC/iQue screening platform from IntelliCyt. The HTFC/iQue Screening System was also used for evaluation of binding to multiple target cell lines or evaluation of unwanted/non-specific binding in parallel, in other words multiplexing. Different cell populations could be distinguished by pre-labelling with distinct amounts of fluorescent dyes, such as Calcein or Cell-Tracker Green, establishing a unique signature of fluorescence intensity for each cell population, thereby producing a fluorescence coding system. The color-coded cell lines were then physically combined and mixed together with antibodies to be tested. Individual cell-lines could be identified via the fluorescence of the respective cell-line that had been pre-labelled. Crude bacterial cell lysates were combined with cells and incubated for 1 hour at room temperature in the dark, shaking gently. Fluorescence measurement was performed with the IntelliCyt HTFC/iQue device. In between incubation steps, no washing was required. Raw data were evaluated with the help of the ‘ForeCyt’ software. After data acquisition, the cell lines from each sample could be identified according to their fluorescence signature and individually evaluated for antibody binding. Staining-conditions for each cell line were optimized in order to find an assay set-up allowing the separation of distinct cell lines (FIG. 5).

Y022065, the parent of the final lead set, resulted from a panning strategy employing captured nanodisc-embedded deglycosylated full length hPAR2 (PAR2-2, 1-377, N30Q, N222Q)_10xH_Sx2. In the first and third round, the antigen was captured with anti-StrepII antibody. In the second round the antigen was captured with Benchmark 1 or coated on a Ni-NTA plate. In summary, specificity was assessed by ELISA-based assessment of binding to FL-StaR nanodisc and truncated StaR nanodisc in presence or absence of ligands AZ8838 and AZ3451; by ELISA-based assessment of binding to counter-targets, including empty nanodiscs, bRIL-His-StrepII, StrepMAB-Immo (capture antibody) and mouse gamma globulin; by assessment of binding to PAR2-expressing cell lines (n>2): CHO V5His_huPAR2, induced HEK Flp-In TRex 293_huPAR2, HEK293F infected BacMam WT FL-huPAR2, A549, Flp-In CHO_ma PAR2 (rhesus, Macaca mulatta), Flp-In CHO_moPAR2 (mouse) as well as PAR2 negative cell lines (parental Flp-In CHO, Jurkat, Flp-In CHO_huPAR1).

### Example 4—Expression and Purification of Fab Fragments and IgG Subcloning into Fab Expression Vector

If required and to facilitate rapid expression of soluble Fab in E. coli, the Fab encoding inserts of the selected Ylanthia® phage were subcloned from pYPdis10 display vector into the Fab-expression vector (pYBex10_h_Fab-FH), comprising the features desired. Subcloning was performed by triple digest via XbaI|EcoRI-HF|PstI-HF.

For full length FabCys expression in HKB11 cells, selected candidates were also cloned into the pYMex10_h_FabCys-AviH expression vector. Subcloning was performed by cutting out the antibody encoding fragments with the restriction enzymes NdeI|XhoI from the pYMex_h_IgG1f_AEASS source vector and ligation into the target vector pYMex10_h_FabCys-Avi-His.

Subcloning into IgG Expression Vector

For full length IgG expression in HKB11 cells, selected candidates were cloned into the pYMex10_h_IgG1f_AEASS expression vector. Subcloning was performed using a method for convenient and efficient conversion of a large amount of sequence-unique Fab clones into the IgG format. In brief, the VH encoding fragment (flanked by restriction sites for NheI|XhoI), the VL encoding fragment (flanked by restriction sites for NdeI|KpnI) and a kappa or lambda specific eukaryotic pYMin expression cassette (flanked by restriction sites KpnI|NheI) were cloned into the acceptor vector for expression in mammalian cells (digested with NdeI|XhoI) in one-step or two-step cloning. After transformation of E. coli XL-1 blue cells, single clones were quality controlled via colony PCR and sequencing of the whole insert region.

For large-scale expression in HKB11 cells, selected candidates were cloned into the pCMex003/004_kan_h_IgG1f_AEASS expression vector, which contains a Zeocin resistance gene for generation of stable pools. In brief, the VH encoding DNA fragment, the VL encoding DNA fragment and the lambda specific eukaryotic pYMin expression cassette were cloned into the acceptor vector pCMex003/004_kan_h_IgG1f_AEASS.

### Generation of Bacterial Lysates Containing Fab Fragments

96-well/384-well microtiter plates pre-filled with growth medium (2×YT containing chloramphenicol, IPTG and 0.1% glucose) were inoculated using glycerol stocks from masterplates. Plates were incubated at 37° C. for bacterial outgrowth and shaken overnight at 22° C. for Fab expression. The next day expression cultures were lysed by addition of BEL buffer containing borate buffer, EDTA and lysozyme. Depending on the selected plate format and application, volumes were adjusted and the protocol for blocking was adapted accordingly. EDTA was omitted if lysates were used for sensitive cell screenings, e.g., functional assays.

### Exploratory Scale Production of Fab Fragments

Expression of Fab fragments encoded by bacterial expression vector in E. coli TG1 F-cells was carried out in shake flask cultures using 500 ml of 2×YT medium supplemented with 0.1% glucose and 34 μg/mL chloramphenicol. Cultures were shaken until the OD600 reached a value of 0.5. Fab expression was induced by adding IPTG (isopropyl-ß-D-thiogalactopyranoside) and further cultivation for 20 hours. Cells were harvested and disrupted using lysozyme. His6-tagged Fab fragments were isolated via IMAC (Bio-Rad|Germany) and eluted using imidazole. Buffer exchange to 1×Dulbecco's PBS (pH 7.2) was performed using ‘PD10’ columns (GE Healthcare|Germany). Samples were sterile filtered (0.2 μm). Protein concentrations were determined by UV-spectrophotometry. The purity of the samples was analysed in denaturing, non-reducing 15% SDS-PAGE. The homogeneity of Fab preparations was determined in native state by size exclusion chromatography (HP-SEC) with calibration standards.

### Microscale Production of IgG in Mammalian Cells

Eukaryotic HKB11 or HEK293-6E cells were transfected with mammalian expression vector DNA encoding both heavy and light chains of IgG. Cell culture supernatants were harvested 7 days post transfection and subjected to Protein A affinity chromatography (MabSelect SURE|GE Healthcare) using a liquid handling station. If not otherwise stated, samples remained in neutralized elution buffer (NaPS: 137 mM Na Phosphate, 81 mM NaCl, pH 7). Samples were sterile filtered (0.2 μm pore size). Protein concentrations were determined by UV-spectrophotometry and purity of IgG was analysed under denaturing, reducing conditions using CE-SDS (LabChip GXII|Perkin Elmer|USA). HP-SEC was performed to analyse IgG preparations in native state.

### Exploratory Scale Production of IgG

Eukaryotic HKB11 or HEK293-6E cells were transfected with mammalian expression vector DNA encoding both heavy and light chains of IgG. Cell culture supernatants were harvested on day 3 or 6 post transfection and subjected to standard Protein A affinity chromatography (MabSelect SURE|GE Healthcare). If not stated otherwise, buffer exchange was performed to 1×Dulbcecco's PBS (pH 7.2|Invitrogen) and samples were sterile filtered (0.2 μm pore size). Protein concentrations were determined by UV-spectrophotometry and purity of IgG was analyzed under denaturing, reducing and non-reducing conditions using CE-SDS (LabChip GXII|Perkin Elmer|USA). HP-SEC was performed to analyse IgG preparations in native state.

### Exploratory Scale Production of AviHis Tagged FabCys

Eukaryotic HKB11 or HEK293-6E cells were transfected with mammalian expression vector DNA encoding both heavy and light chains of disulfide-bridged FabCysAviHis. Cell culture supernatants were harvested on day 3 or 7 post transfection and subjected to metal ion affinity chromatography (Protino Ni-NTA|Macherey Nagel). If not stated otherwise, buffer exchange was performed to 1×Dulbecco's PBS (pH 7.2|Invitrogen) and samples were sterile filtered (0.2 μm pore size). Protein concentrations were determined by UV-spectrophotometry and purity of FabCysAviHis was analysed under denaturing, reducing and non-reducing conditions using CE-SDS (LabChip GXII|Perkin Elmer|USA). HP-SEC was performed to analyse FabCysAviHis preparations in native state.

### Igg Production for In Vivo Characterization

Material production was undertaken by establishing stable HKB11 cell pools. Eukaryotic HKB11 cells were transfected with mammalian expression vector DNA encoding both heavy and light chains of IgG. For generation of stably expressing cell pools, respective vectors additionally contain a Zeocin resistance gene. Three days post-transfection selection was started by the addition of 160 μg/ml Zeocin to the cell suspension. During selection, cell count and viability initially decreased. 20 to 30 days after transfection cells started to recover. Reaching a viability of ˜80%, stable pools were scaled up to the desired volume. Cell culture supernatants were harvested on day 6 post transfection and subjected to Protein A affinity chromatography (MabSelect SURE|GE Healthcare). If needed, a second purification step (preparative SEC|Superdex 200|GE Healthcare) was performed to remove aggregates. Buffer exchange was performed to 1×Dulbecco's PBS (pH 7.2|Invitrogen) and samples were sterile filtered (0.2 μm pore size). Protein concentrations were determined by UV-spectrophotometry and purities of IgG were analyzed under denaturing, reducing and non-reducing conditions using CE-SDS (LabChip GXII|Perkin Elmer|USA). HP-SEC was performed to analyze IgG preparations in native state. Endotoxin levels were determined by KQCL assay (Lonza). Protein identities were confirmed using Mass Spectrometry analysis (100MDL43).

### Example 5—Lead Isolation Clone Profiling

### Cell-Based IP-One Gag Assay at Human PAR2 for Identification of Functional Primary Hits

Functional characterization was performed after full validation of clones in ELISA and IntelliCyt to confirm PAR2 binding status. Exploratory scale purified IgG1f_AEASS clones were tested for their ability to inhibit activation of the PAR2 receptor by the synthetic agonist, the Activating Peptide (2-Furyol-LIGRO) using the Cisbio® IP-One Gαq assay at two concentrations in replicate.

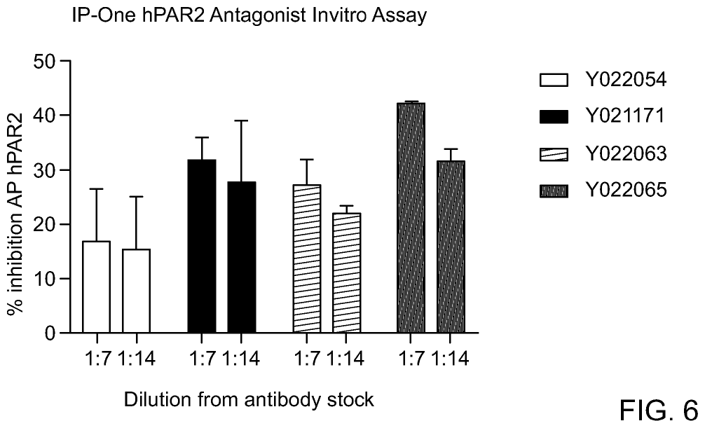

HEK293F cells were infected with a 2% v/v human PAR2 BacMam virus in the presence of 0.5 mM sodium butyrate in growth media (Pro293, 5% FBS, 1% Glutamax, 0.4% penicillin/streptomycin) for 24h cultured in suspension format in a humidified incubator with 5% CO2 atmosphere at 37° C. On experiment day, cells were harvested and re-suspended at a density of 1×10\hat{ }6/ml in assay buffer at a density of 1×10\hat{ }6/ml (1 part stimulation buffer (Cisbio): 5 parts ddH20) containing LiCl with 0.5% bovine serum albumin (BSA, Sigma). Antibodies were tested from stock concentration or following an initial 1:2 dilution in PBS. A Sul sample of either concentration of antibody in replicate was added to a half area white 96-well plates (Corning) followed by 25 μl PAR2 of cell suspension. Plates were incubated at 37° C. for 30 minutes prior to addition of 5 μl/well Activating Peptide for a final agonist challenge concentration of 6.28 nM. The final assay antibody screening was performed at a dilution of either 1:7 or 1:14 from Ab stock. Plates were re-incubated for a further 30 minutes at 37° C. prior to addition of 10 μl IP-One detection kit (Cisbio) in lysis buffer. Cell plates were placed on a plate shaker at room temperature (RT) for 1h before reading plates on the PHERAstar FS microplate reader (BMG Labtech) using standard HTRF protocols, with excitation at 335 nm and emissions read at both 620 nm and 665 nm. HTRF ratios were calculated as in equation 1. Responses were normalised for % inhibition of Activating Peptide IP-One response at 6.28 nM (EC₈₀) final assay concentration (FIG. 6).

### Equation 1: Calculation of HTRF Ratios

$$HTRF Ratio = \frac{Emission at 665 nm}{Emission at 620 nm} \times 10 , 000$$

### Table 2: Inhibition of Peptide-Induced Agonism of Human PAR2

Functional characterization of Y021171, Y022063 and Y022054 & Y022065 hIgG1f_AEASS in the peptide inhibition assay using the Cisbio® IP-One Gαq assay at the highest concentration in replicate. Also shown in FIG. 6.

| Antibody clone ID | % Max Inhibition AP ± SD | Number of repeats |
|---|---|---|
| Y022054 | 17 ± 10 | 2 |
| Y021171 | 32 ± 4 | 2 |
| Y022063 | 27 ± 5 | 2 |
| Y022065 | 43 ± 1 | 2 |

### Affinity Determination

### K_{D}, ka and kd Determination by SPR Using Antibody Capture Setup

For KD determinations, monomer fractions of antibody protein (Fab fragments or IgG) were used (at least 90% monomer content, as analysed by analytical SEC). Affinities were determined by kinetic characterization using either Biacore (Biacore T200) or Octet (QK384 or HTX) instruments as described below.

The antibodies were captured on anti-human IgG chip surface followed by injection of 100 nM PAR2. The data were fitted to 1:1 interaction model except for clone ‘Y021160’ in which case heterogeneous ligand model was used and component with slow dissociation was used for comparison.

### Table 3: Biacore of Lead Isolation Clones

| Antibody clone ID | ka (1/Ms) | kd (1/s) | K_{D} (M) |
|---|---|---|---|
| Y022065 | 3.0E+04 | 1.6E+04 | 5.3E−09 |
| Y021172 | 3.5E+09 | 2.7E+02 | 7.7E−08 |
| Y021194 | 4.6E+04 | 1.4E−03 | 3.0E−08 |
| Y021160 | 2.0E+05 | 6.3E−04 | 3.1E−09 |
| Y021171 | 4.1E+05 | 2.9E−02 | 7.0E−08 |

An appropriate high-capacity capture surface was prepared, e.g. by covalently immobilizing an appropriate capture ligand onto a CM5 chip (Biacore, GE Healthcare) or AR2G Sensors (forteBIO, Pall Corp.) using EDC/NHS chemistry, or by loading SA sensors with biotinylated capture reagents. Examples for appropriate capture systems were anti-hu-Fc antibody (Biacore, GE Healthcare), Protein A sensors (forteBIO, Pall Corp.), MabSelect Sure Ligand (GE Healthcare), and anti-His-tag antibody (Genscript). Six to eight different analyte concentrations (2ⁿ serial dilution) were used for analysis during kinetic experiments. After each cycle, the sensor surface was regenerated to remove captured antibody/antigen complexes, while maintaining the integrity of the capture surface. A blank injection of running buffer was used for referencing. In all experiments, the assay buffer was matched to the formulation of the PAR2 protein, i.e. base buffer and the detergent. The IgGs were captured via the Fc-fragment and detergent (LMNG/CHS) solubilised FL-N PAR2 StaR (PAR2-3) was used as analyte in solution. 50 mM HEPES pH 7.5, 150 mM NaCl, 0.02% LMNG was used as assay buffer. CM3 sensor chips were used as basis to immobilize a high density of anti-hu-Fc capture antibody (BR-1008-39, GE Healthcare). IgGs were captured with relatively high levels of approx. 500 RU to achieve a higher ratio of specific to non-specific binding (while still maintaining conditions suitable for kinetic characterization). Sensorgrams were evaluated with the corresponding instrument's evaluation software, i.e. Biacore T200 Evaluation Software 3.x (Biacore, GE Healthcare) or fortéBIO Octet Data Analysis (forteBIO, Pall Corp.). All sensorgrams were fitted to a 1:1 binding model to determine ka and kd rate constants, which were used to calculate K_{D}. With Y021160, the heterogeneous ligand model was applied and component with slow dissociation was used to calculate ka, kd, and K_{D}.

For three purified IgG1f_AEASS clones Y021171, Y022054 and Y022063 affinities to human PAR2-3 variant (P157: 1-377, glycosylated, pBacMamHisStrepII, full length) were determined by SPR immobilizing the IgG and probing with detergent (LMNG/CHS) solubilized FL-N PAR2 StaR. Affinities were 22 nM and 20 nM for Y022063 and Y021171, respectively. Y022054 displayed a 10-fold higher affinity of 2.5 nM compared to the other candidates. Y021171 showed a fast association (ka) but also a very fast dissociation (kd). Extrapolating from binding affinities towards functional inhibition, the required affinity of an antagonistic anti-PAR2 antibody was assumed to be in the <100 pM range. An “off rate selection” strategy (Hawkins et al. 1992) was implemented during the affinity maturation campaign to improve K_{D} properties, notably for Y021171.

The assay of PAR2 interaction with anti-PAR2 antibodies was carried out using a Biacore T200 instrument (GE Healthcare). Anti-human IgG antibody (Human Antibody Capture Kit, GE Healthcare) was immobilised by amine coupling on a sensor chip CM3 (GE Healthcare) to a surface density of 3500 to 4500 resonance units (RU). Immobilisation was carried out at 25° C. in HBS-EP+ buffer (GE Healthcare). The buffer was then changed to 50 mM HEPES, pH 7.5, 150 mM NaCl, 0.02% LMNG, 0.002% CHS. Each cycle of the PAR2-antibody interaction assay consisted of antibody capture (100 nM antibody injected for 2 min at a flow rate of 5 μL/min), PAR2 injection (blank or 250 nM, with contact and dissociation times of 2 and 3 minutes, respectively, at a flow rate of 20 μl/minute), and chip surface regeneration (30 seconds at 20 μl/minute) using the regeneration solution from Human Antibody Capture Kit (GE Healthcare). The blank-subtracted data were fitted to 1:1 interaction model to obtain association and dissociation rate constants, ka (kon) and kd (koff), and the affinity constant K_{D}. Measurements between each experimental data set were within the acceptable margins for this methodology.

### Table 4: Affinity Determination of Functional Candidates Y021171, Y022063 and Y022054 by SPR

The antibodies were captured on anti-human IgG chip surface followed by injection of 250 nM PAR2. The data were fitted to 1:1 interaction model.

| Clone | ka (1/Ms) | kd (1/s) | K_{D} (M) |
|---|---|---|---|
| Y022054 | 1.2E+05 | 2.9E−04 | 2.5E−09 |
| Y021171 | 5.3E+05 | 1.1E−02 | 2.0E−08 |
| Y022063 | 1.8E+05 | 3.8E−03 | 2.2E−08 |

### Example 6—Affinity Maturation/Lead Optimisation and Phage Selections

Several IgG clones, including Y021171, Y022054, Y022063 and Y022065 partially inhibited PAR2-AP activation in the IP One assay. Several other clones, but none of the four functional candidates, were shown to be cross-reactive to non-human primate (NHP) PAR2 and despite a high degree of identity amongst human and rhesus PAR2 sequences in the extracellular domains, none of the clones displayed clear cross-reactivity to FlpIn CHO_maPAR2 (rhesus, Macaca mulatta) nor Flpln CHO_moPAR2 (mouse) before affinity maturation.

### Maturation Libraries

Generation of phage maturation libraries: The cloning of the maturation libraries was performed in the CysDisplay™ vector encoding for the parental Fab fragments. If not already present in the CysDisplay™ vector, the DNA sequences encoding for the parental Fab fragments were transferred into the respective vector via restriction digest and ligation prior to library cloning. To increase affinity and biological activity and to reduce non-specificity of selected antibody candidates, CDR-L3 and CDRH-1/CDR-H2 regions were optimized in parallel. Owing to their modular construction, MorphoSys' antibody libraries are designed for affinity optimization by CDR exchange, where single CDRs of antibodies are excised and replaced by individual members of large CDR libraries (Prassler et al. 2009). Ylanthia® maturation modules (YMM) are based on the Ylanthia library design and were prebuilt with the Slonomics® technology (van den Brulle et al. 2008). The generation of the maturation libraries was performed for each maturation candidate individually. In order to monitor the cloning efficiency, the parental CDR-L3 is replaced by an Ylanthia Maturation Stuffer (YMS), before the diversified LCDR-L3 YMM is inserted. Digested vector fragments were ligated with a 2-fold molar excess of the insert fragment carrying the diversified CDR-L3s. The same procedure was applied for diversification of CDRH-1 & CDR-H2. Ligation mixtures were electroporated in E. coli MC1061F′ cells yielding in >108 independent colonies. Amplification of the library was performed as described in the literature (Tiller et al. 2013). For quality control, approximately 10 single clones per library were randomly picked and Sanger sequenced.

Maturation candidates were selected according to the following scientific rationale and criteria:
1. Deselection of candidates with increased risk of off-target binding due to elevated binding on:
   PAR2-negative antigens (ELISA): Empty nanodiscs, Mouse IgG, anti-StrepII, bRIL
   PAR2-negative cell lines (FACS): parental Flp-In CHO cells, Flp-In CHO_huPAR1 and Jurkat cells
2. Deselection of candidates preferentially binding to the membrane distal part of the N-terminus (assessed by comparative ELISA on FL-StaR nanodisc versus truncated StaR nanodisc)
Human PAR2-specific IgG1f_AEASS candidates were ranked as follows:
   1. Functionally active candidates;
   2. Binding to cell surface expressed human PAR2 with a focus on the functionally active HEK293F cell line infected with WT FL-huPAR2 BacMam as a prerequisite for assessment of functionality;
   3. Competition of binding with the functionally active candidate Y021171 candidate and/or the functionally active R&D anti-PAR2 reagent mAb MAB3949 because the published PAR2 structure (Cheng et al., 2017) demonstrated that this reagent antibody interacted with the extracellular loops.

### Table 5: Overview of Maturation Candidates

| | | Competition | | Human | Rhesus |
|---|---|---|---|---|---|
| Antibody | | with | Competition | PAR2 | PAR2 |
| Clone ID | VH/VL | R&D mAb | with Y021171 | binding | binding |
| Y021171 | VH3-21/ | Not | Reference | Yes | No |
| | Vκ1-12 | determined | | | |
| Y022054 | VH3-07/ | No | Yes | Yes | No |
| | Vκ1-12 | | | | |
| Y022063 | VH3-23/ | Yes | No | Yes | No |
| | Vλ2-23 | | | | |
| Y022065 | VH3-23/ | Yes | Yes | Yes | No |
| | Vλ2-23 | | | | |

The primary goal of affinity maturation is the improvement of an existing feature and does usually not lead to a displacement of the targeted epitope. It was observed in IgG characterizations that functional candidates were not cross-reactive to rhesus PAR2, whereas non-functional candidates bound to this species. Due to the lack of recombinant rhesus PAR2 StaR antigen material, a differential whole cell panning strategy (DWCP) was implemented.

SPR data highlighted a fast off-rate (kd (1/s) of 0.01) for Y021171. Therefore, during the third maturation panning round, a so called “off-rate selection” procedure (Hawkins et al., 1992) was implemented to enrich candidates with improved off rates. As SPR data was not available for all maturation candidates, this method was restricted to panning strategies including functional candidates only.

During maturation, washing stringency was increased over panning rounds, e.g., by increasing the number and duration of washing steps (e.g. overnight washing). Prolonged or overnight washing was used in off-rate selections strategies in combination with the addition of soluble antigen to the washing buffer to prevent any rebinding of antibody-phage to the immobilized antigen.

Nine parental clones were progressed into an affinity maturation campaign covering all available binding profiles and potential modes of action. To increase affinity and biological activity of previously selected antibody fragments, CDR-L3 or CDR-H1 & CDR-H2 regions were exchanged in parallel by diversified modules (Prassler et al. 2009). Fixed VH/VL human germline framework pairs were preserved using specific Ylanthia® maturation modules (YMM) to avoid the emergence of cross-clones or additional framework combinations. Parental Fab fragments were transferred from the corresponding expression vector into the CysDisplay™ vector prior to library cloning for affinity maturation. CDR-L3 and CDR-H1 & CDR-H2 libraries were cloned separately for each maturation candidate and pooled. Affinity maturation was tailor made for 6 individual candidates, namely all functional candidates (Y021171, Y022054, Y022063, Y022065), as well as Y022059 and Y022069. Weaker candidates were matured in a pool (Y021160, Y022075 and Y022079). Maturation libraries were generated for individual diversification of CDR-L3 and CDR-H1 & CDR-H2. Maturation pannings were designed with an emphasis on high stringency solid phase pannings using full length or N-terminally truncated nanodisc-embedded PAR2 StaRs, as well as on the generation or improvement of rhesus PAR2 cross-reactivity. Following affinity maturation and screenings, the project team was successful in identifying a large set of highly specific Ylanthia® antibody candidates. Clones were progressed into IgG conversion and in-depth characterization.

### Affinity Maturation Panning Selections

To increase affinity and biological activity of previously selected antibody fragments, CDR-L3 and CDR-H2 regions were exchanged in parallel by diversified modules (Prassler et al. 2009). Parental Fab fragments were transferred from the corresponding expression vector into the CysDisplay™ vector prior to library cloning for affinity maturation. For the selection of candidates that are affinity improved, phage derived from maturation libraries were subjected to three rounds of maturation panning. Panning stringency was increased by lowering the antigen concentration in each panning round (Low et al. 1996). In addition to antigen reduction, off-rate selections were performed (Hawkins et al. 1992) for selected strategies. These strategies were combined with prolonged washing steps in combination with the addition of soluble antigen to the washing buffer to prevent any rebinding of antibody-phage to the immobilized antigen.

After affinity maturation, the DNA fragment coding for the modified variable region was directly replaced in the pYMex10_h_IgG1f_AEASS expression vector, which codes for the parental IgG. In brief, the vector component coding for the parental VH or VL was removed with appropriate restriction enzymes (NheI|XhoI for VH, NdeI|KpnI for VL) and a fragment coding for the affinity matured variable region was inserted and expressed and purified as previously described to generate full length IgG.

### Example 7—Lead Optimisation and Clone Profiling

Profiling of purified Ylanthia® IgGs included binding specificity and cross-reactivity assessment to human, mouse, rhesus and marmoset PAR2-expressing cells, as well binding to PAR2 re-confirmed by ELISA to nanodisc-embedded StaRs and by flow cytometry to PAR2 expressing cells. Following affinity maturation, several IgG clones, mostly deriving from the functionally active parental IgG Y022065, were confirmed with antagonistic activity due to their ability to fully inhibit human PAR2 receptor activation and thus emphasizing their specificity for a functionally relevant epitope.

Primary screening on truncated or FL PAR2 StaR nanodiscs in ELISA revealed an excellent improvement of binding compared to the parental antibody. The kd-ranking ELISA was performed for all panning subcodes. Plotting of the data gained with the classical ELISA setting versus the Koff-ranking ELISA setting showed a clear linear correlation of signals, indicating that most matured candidates had a slow Koff value. For analysis, loss of binding signal was calculated as the ratio between the signal over background (S/BG) of the classical ELISA setting and the S/BG of the Koff-ranking ELISA: [truncated/FL PAR2 StaR nanodisc S/BG]/[Koff ELISA truncated/FL PAR2 StaR nanodisc S/BG]. A poor Koff estimate according to an ELISA ranking correlated with low signals in the classical ELISA setting. Candidates with a ratio >2-fold loss in signal were classified as candidates with a potentially fast off-rate. 94% of matured candidates displayed no or low change in signal (<2-fold), 67% thereof were derivatives of the parental candidate Y021171.

Parental Y021171 displayed the largest loss of binding signal in the Koff-ranking ELISA whereas other parental antibody candidates displayed no or low change in signal. The result was in accordance with previous SPR data generated for Y021171 where the candidate was ascribed a fast off-rate of kd (1/s)=0.01058. 6% of matured candidates displayed an elevated change in signal (2-4.5-fold) suggestive of a fast off-rate. 92% thereof were matured derivatives of Y021171 (˜33% of total Y021171 candidates) (FIG. 7).

### Whole Cell Binding to PAR2 as Determined by IntelliCyt

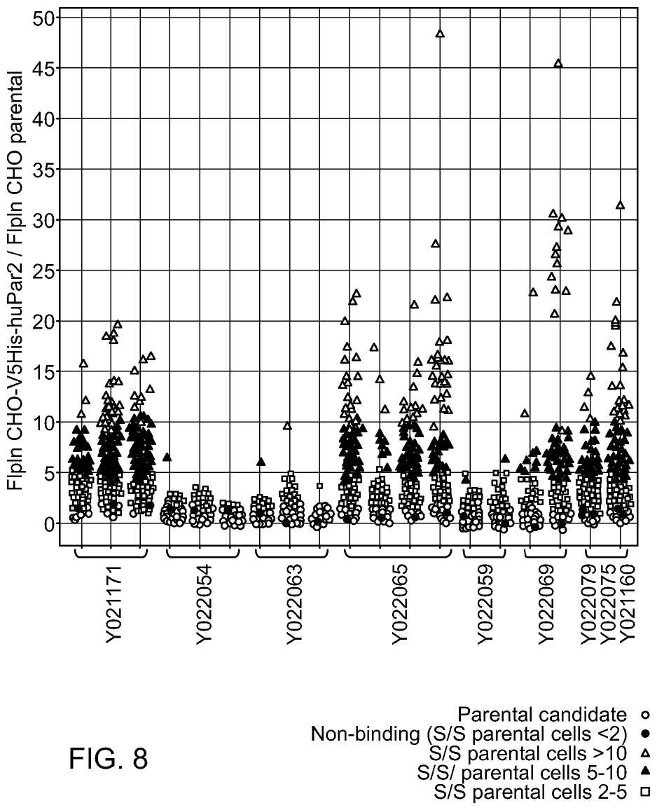

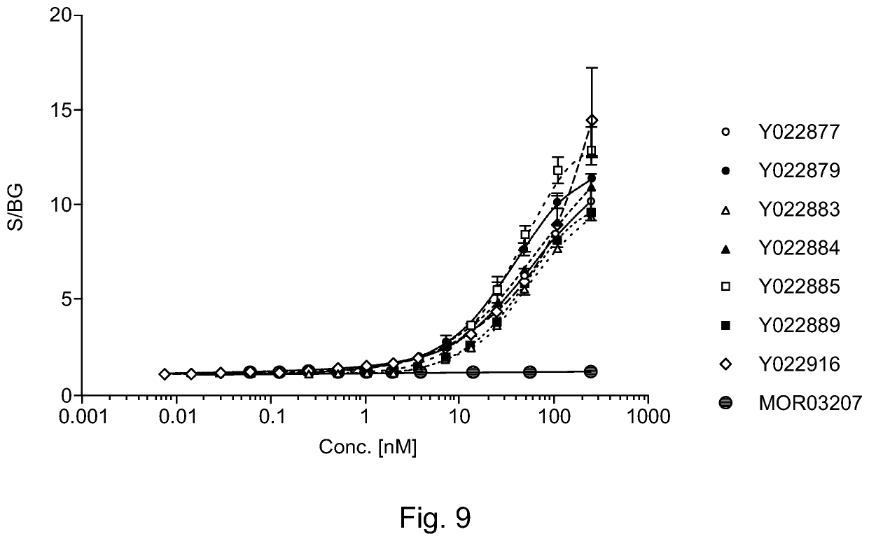

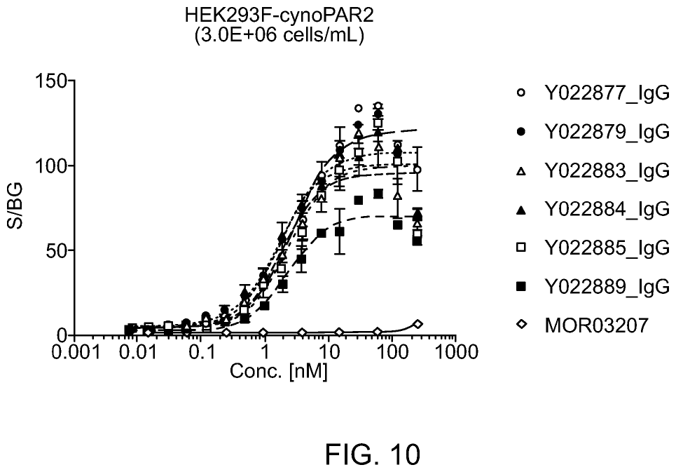

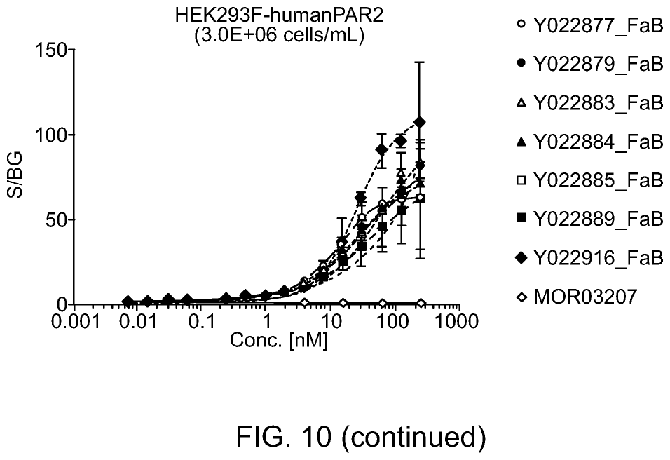

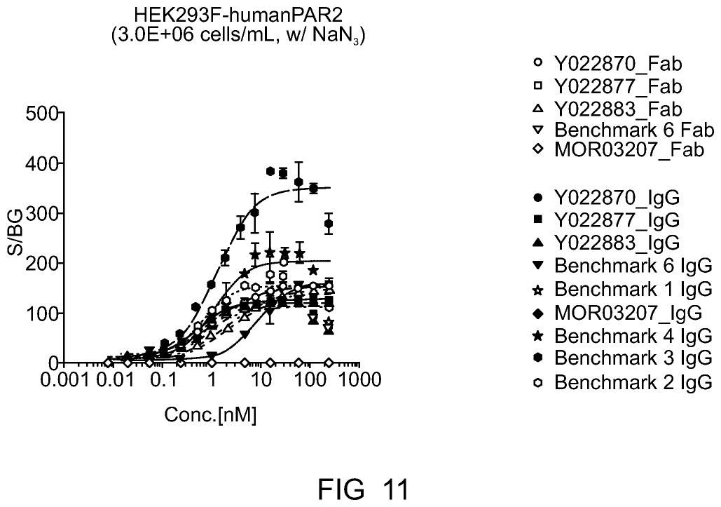

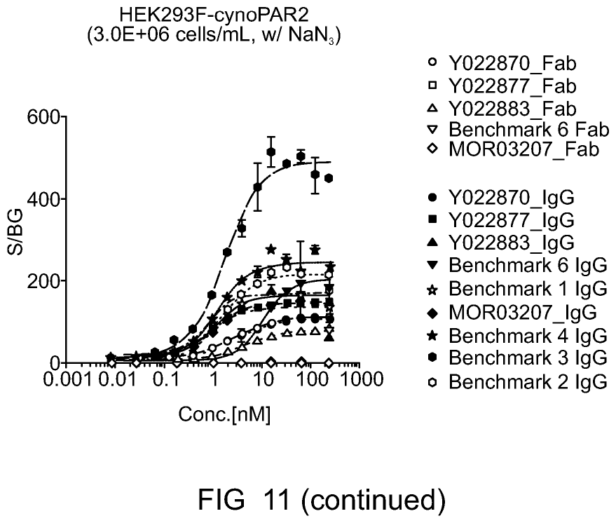

Screening of 2200 primary candidates on Flpln CHO-V5His-huPAR2 versus FlpIn CHO parental cells in Intellicyt revealed an improvement of binding for most matured derivatives compared to their respective parental antibody candidate. Best improvement was achieved for maturation pannings of Y021171, Y022065, Y022069 and parentals matured in pool, with derivatives of Y022079 expected to be the predominant family. All candidates displayed a low binding on FlpIn CHO parental cells (S/BG<1.5). Over 800 candidates displayed good or excellent binding to FlpIn CHO-V5His-huPAR2 with a 2-5-fold, 5-10-fold or >10-fold improvement of binding over Flpln CHO parental cells (Signal/Signal parental) (FIGS. 8-11).

### Protein Panel Profiling (3P) for PAR2 Specificity

For protein panel profiling (Frese et al. 2013), 32 different proteins and controls were coated on two 384-well MSD standard plates (Meso Scale Discovery, MTP 384-well MA6000, #L21XA) at a concentration of 1.0 μg/mL at 4° C., overnight. The coating solution was discarded and plates were blocked with 50 μL 3% (w/v) BSA or 3% (w/v) skim milk powder in PBS for one hour at room temperature on a microtiter plate shaker (˜500 rpm) followed by three washing steps with 50 μl washing buffer (PBS with 0.05% (v/v) Tween 20). Antibody samples (Fab fragment or IgG) were diluted to 100 nM and 10 nM in assay buffer (PBS with 0.5% (w/v) BSA, 0.05% (v/v) Tween 20). As controls, MOR reference mAb anti-lysozyme MOR03207 Fab or IgG (depending on the sample format) and assay buffer were used. Samples and controls were added (30 μL/well) and incubated for three hours at room temperature on a microtiter plate shaker. The plates were washed three times and 30 μL detection antibody (ECL-labelled anti-human Fab, diluted 1:2000) was added per well and incubated for one hour on a microtiter plate shaker (˜500 rpm). After washing the MSD plate and adding 35 μL/well MSD Read Buffer T with surfactant, electrochemiluminescence signals were detected using a Sector Imager 6000 (Meso Scale Discovery|Gaithersburg|MD|USA). For evaluation, signals of the antibody sample on a certain protein were divided by the respective signals of the anti-lysozyme reference mAb MOR03207 resulting in a binding ratio. Most clones, including all later maturation clones, displayed no or low non-specific binding propensity.

### Example 8—Functional Characterisation of Lead Optimized Clones

### Functional Assay: Cell-Based IP-One Gαq Assay

Assay methodology follows that of example 5. Antibody stocks were serially diluted 1:2 over a 10-point concentration curve in order to determine an IC₅₀ value. Lead optimized antibody clones were tested in challenge against Activating Peptide (2-Furyol-LIGRO, 6.28 nM; Table 6), Bovine Trypsin (2 nM; Table 7) or PAR1 Activating Peptide (SFLLR-NH2, 632 nM; Table 8) in separate experiments.

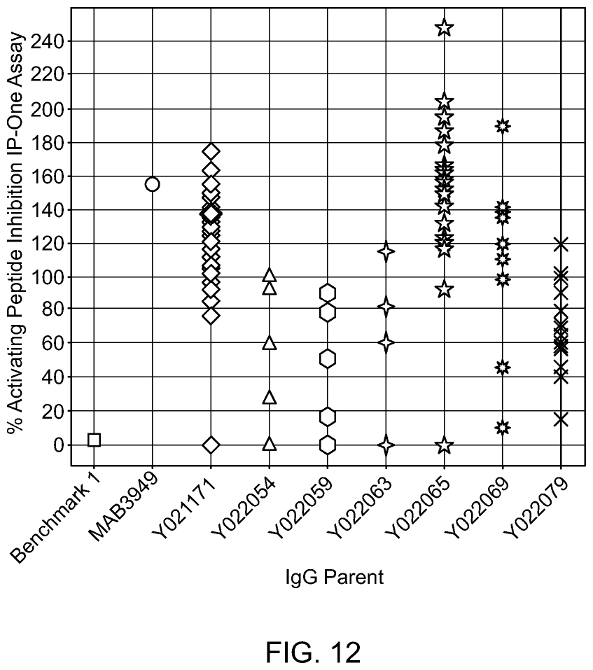

IP-One HTRF results are normalised to effect of 10 μM MAB3949 from when screening parental antibody clones in challenge against Activating Peptide (FIG. 12)

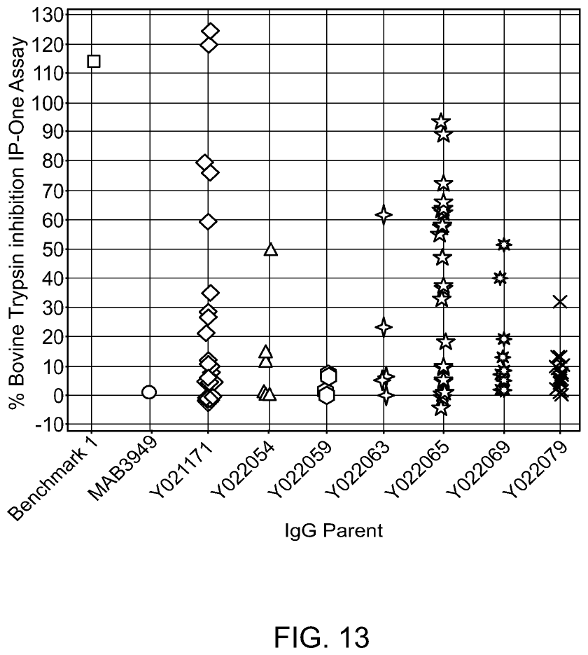

IP-One HTRF results are normalised to effect of 1 μM Benchmark 1 from when screening parental antibody clones in challenge against Bovine Trypsin (FIG. 13)

IP-One HTRF results are normalised to effect of 10 μM MAB3949 when screening in challenge against PAR1 peptide SFFLR-NH2 Trypsin (Table 8)

Normalised data was fitted in GraphPad Prism version 7.04 to a 4-parameter sigmoidal dose-response curve (Equation 2).

The data in Table 8 shows that the Y022883 antibody is capable of blocking PAR2 activation by the PAR1 Activating Peptide.

### Equation 2: 4 Parameter Sigmoidal-Dose Response Fit

$$Y = Bottom + ( Top - Bottom ) / ( 1 + ( ( X ⋀ HillSlope ) / ( lC 50 ⋀ HillSlope ) ) )$$

Where IC₅₀ is the concentration (in nM) which inhibits the AP/Trypsin response by 50%, and % maximum inhibition values is the maximal inhibition of AP/Trypsin (taken from the minimum asymptote of the curve i.e. ‘bottom’).

Table 6: Functional characterization of lead optimized clones against Activating Peptide that demonstrate activity in replicate in the IP-One human PAR2 antagonist assay using the Cisbio® IP-One Gαq kit which also share activity in Bovine Trypsin challenge assay.

| | | Human PAR2 | % Max | |
|---|---|---|---|---|
| Parent | | AP IC₅₀ | Inhibition ± | Number of |
| clone | Lead clone | (nM) ± SD | SD | repeats |
| Benchmark 1 | | inactive | 3 ± 1 | 2 |
| MAB3949 | | 241 ± 60 | 100 ± 6 | 2 |
| Y021171 | Y022931 | 56 ± 22 | 155 ± 64 | 2 |
| Y021171 | Y022930 | 113 ± 48 | 116 ± 48 | 2 |
| Y021171 | Y021171 | 4254 ± 360 | 163 ± 124 | 2 |
| Y022063 | Y022063 | 2392 ± 1717 | 61 ± 56 | 2 |
| Y022065 | Y022883 | 57 ± 11 | 149 ± 8 | 2 |
| Y022065 | Y022885 | 56 ± 3 | 142 ± 2 | 2 |
| Y022065 | Y022884 | 51 ± 7 | 150 ± 12 | 2 |
| Y022065 | Y022889 | 57 ± 11 | 131 ± 7 | 2 |
| Y022065 | Y022870 | 55 ± 7 | 203 ± 76 | 2 |
| Y022065 | Y022876 | 62 ± 0 | 156 ± 4 | 2 |
| Y022065 | Y022860 | 57 ± 1 | 155 ± 2 | 2 |
| Y022065 | Y022879 | 61 ± 0 | 161 ± 6 | 2 |
| Y022065 | Y022857 | 63 ± 3 | 186 ± 50 | 2 |
| Y022065 | Y022887 | 58 ± 5 | 143 ± 15 | 2 |
| Y022065 | Y022858 | 73 ± 11 | 161 ± 25 | 2 |
| Y022065 | Y022881 | 58 ± 3 | 158 ± 10 | 2 |
| Y022065 | Y022882 | 49 ± 8 | 124 ± 28 | 2 |
| Y022065 | Y022877 | 63 ± 12 | 167 ± 1 | 2 |
| Y022065 | Y022880 | 61 ± 0 | 178 ± 39 | 2 |
| Y022079 | Y022878 | 119 ± 32 | 102 ± 67 | 2 |

Table 7: Functional characterization of lead optimized clones against Bovine Trypsin that demonstrate activity in the IP-One human PAR2 antagonist assay using the Cisbio® IP-One Gαq kit which also share activity in Activating Peptide challenge assay. Where activity could only be confirmed in one replicate, this is represented in the column labelled ‘Number of repeats’.

| | | Human PAR2 | % Max | |
|---|---|---|---|---|
| | Lead | Trypsin IC₅₀ | Inhibition ± | Number of |
| Parent | clone | (nM) ± SD | SD | repeats |
| Benchmark 1 | | 44 ± 0 | 118 ± 5 | 2 |
| MAB3949 | | inactive | 0 | 2 |
| Y021171 | Y022931 | 3365 ± 0 | 27 | 1 |
| Y021171 | Y022930 | 332 ± 0 | 50 | 1 |
| Y021171 | Y021171 | 54 ± 0 | 19 | 1 |
| Y022063 | Y022063 | 58 ± 0 | 31 | 1 |
| Y022065 | Y022883 | 183 ± 161 | 48 | 1 |
| Y022065 | Y022885 | 226 ± 219 | 90 | 1 |
| Y022065 | Y022884 | 204 ± 187 | 73 ± 30 | 2 |
| Y022065 | Y022889 | 279 ± 0 | 70 | 1 |
| Y022065 | Y022870 | 72 ± 0 | 10 ± 0 | 2 |
| Y022065 | Y022876 | 82 ± 36 | 34 | 1 |
| Y022065 | Y022860 | 70 ± 0 | 22 ± 0 | 2 |
| Y022065 | Y022879 | 101 ± 18 | 63 ± 29 | 2 |
| Y022065 | Y022857 | 66 ± 0 | 20 ± 0 | 2 |
| Y022065 | Y022887 | 255 ± 256 | 64 | 1 |
| Y022065 | Y022858 | 69.5 ± 28 | 115 ± 15 | 1 |
| Y022065 | Y022881 | 161 ± 0 | 9 | 1 |
| Y022065 | Y022882 | 169 ± 28 | 64 | 1 |
| Y022065 | Y022877 | 89.5 ± 43 | 58 ± 24 | 2 |
| Y022065 | Y022880 | 161 ± 104 | 94 ± 9 | 2 |
| Y022079 | Y022878 | 99 ± 0 | 106 | 1 |

Affinity matured lead clones derived from parent IgG Y022065 display the largest proportion of active lead clones active against both Activating Peptide and Trypsin. Tables 6 and 7 list functional IgG actives which demonstrate IC₅₀ values n≥1 against both Activating Peptide and Bovine Trypsin.

Table 8: IP-One HTRF Results are Normalised to Effect of 10 μM MAB3949 when Screening in Challenge Against PAR1 Peptide SFFLR-NH2 Trypsin.

| | | Human PAR2 | % Max | |
|---|---|---|---|---|
| Parent | | SFLLR IC₅₀ | Inhibition ± | Number of |
| clone | Lead clone | (nM) ± SD | SD | repeats |
| Benchmark 2 | | inactive | 3 ± 1 | 2 |
| Y022065 | Y022883 | 13.5 ± 12.5 | 100 ± 5 | 2 |

### Functional Assay: Cell-Based FLIPR Calcium Mobilisation Gαq Assay Using HT-29 Cell Line

Confirmation of activity in an immortalised human colonic adenocarcinoma cell line (HT-29 endogenously expressing the PAR2 receptor was undertaken after IC₅₀ confirmation of lead clones in the IP-One recombinant human PAR2 in vitro assay.

HT-29 cells (ATCC HTB-38 were kept in continuous culture using DMEM medium with high glucose (25 mM), without sodium pyruvate, but with GlutaMAX (Gibco, Paisley, UK), 10% of heat-inactivated fetal bovine serum and penicillin/streptomycin (100 units/mL of penicillin and 100 μg/ml of streptomycin) in a humidified incubator with 5% CO2 atmosphere at 37° C. Culture medium is changed every 2 days from the second day after seeding, and cells are harvested in the logarithmic phase of growth after reaching 80-90% confluency by 0.05% trypsin/EDTA.

Cells were plated at 50 μL/well in culture media at a cell density of 5,000 cells per well in 384-well black wall plates (Corning) and incubated for 24 hours in a humidified incubator with 5% CO2 atmosphere at 37° C. On experiment day, cell media was removed and 50 μl assay buffer (HBSS 20 mM HEPES PH7.4 buffer containing 0.1% BSA) containing Calcium 5 dye at a 1:20 dilution from stock (Molecular Devices). Plates were re-incubated at 37° C. for 45 minutes prior to cells equilibrated for a further 15 minutes at room temperature.

IgG lead clones were prepared in assay buffer and serially diluted to generate a 10-point curve. IgG dose response curves were added online using the FLIPR Tetra (Molecular Devices) pipettor (10 μL) and the calcium response measured over a 5-minute period. Plates were re-incubated again at 37° C. for 60 minutes prior to a 10 μL/well online addition of either Activating Peptide (630 nM) or Bovine Trypsin (63 nM) and further measurement of human PAR2 activated by calcium mobilisation conducted over a 5 minute period.

Data was analysed by extraction Max-Min raw data files and analysed by Equation 2. Data was normalised against MAB3949 or Benchmark 1 for Activating Peptide and Bovine Trypsin assays, respectively.

Table 9: Functional Characterization of Lead Clones Against Bovine Trypsin or Activating Peptide (AP) that Demonstrate Full Dose Dependent Inhibition n>3 in the HT-29 FLIPR Calcium Mobilization Assay Normalized Against Corresponding Antibody Control.

| | | Trypsin IC₅₀ | AP IC₅₀ |
|---|---|---|---|
| Parent clone | Lead clone | (nM) ± SD | (nM) ± SD |
| Y022059 | Y022856 | 60 ± 38 | 76.2 ± 55 |
| Y022065 | Y022857 | 51 ± 58 | 20.5 ± 55 |
| Y022065 | Y022858 | 104 ± 58 | 62.8 ± 60 |
| Y022065 | Y022860 | 138 ± 44 | 62.6 ± 60 |
| Y022065 | Y022861 | 159 ± 38 | 70.7 ± 53 |
| Y022065 | Y022867 | 171 ± 41 | 87.5 ± 47 |
| Y022065 | Y022870 | 40 ± 46 | 17.8 ± 51 |
| Y022065 | Y022877 | 49 ± 77 | 44.3 ± 71 |
| Y022065 | Y022879 | 42 ± 64 | 34.6 ± 75 |
| Y022065 | Y022880 | 107 ± 61 | 67.9 ± 60 |
| Y022065 | Y022881 | 35 ± 64 | 23 ± 56.8 |
| Y022065 | Y022882 | 67 ± 59 | 38.2 ± 78 |
| Y022065 | Y022883 | 47 ± 41 | 42 ± 79 |
| Y022065 | Y022884 | 116 ± 39 | 39.4 ± 73 |
| Y022065 | Y022885 | 65 ± 69 | 76.7 ± 58 |
| Y022065 | Y022887 | 192 ± 48 | 51.5 ± 35 |
| Y022065 | Y022889 | 94 ± 48 | 64.1 ± 61 |

### Functional Assay: Cell-Based IP-One Gag Assay at Cynomolgus PAR2 and Rhesus PAR2

Functional characterization was performed to test lead clone cross-species antagonist activity against Cynomolgus and rhesus PAR2 receptor in challenge against synthetic agonist, SLIGKV using the Cisbio® IP-One Gαq assay at two concentrations in replicate.

HEK293f cells were infected with a 2.5% v/v Cynomolgus PAR2 BacMam virus, or a 2.5% v/v Rhesus PAR2 BacMam virus, in the presence of 0.5 mM sodium butyrate in growth media (Pro293, 5% FBS, 1% Glutamax, 0.4% penicillin/streptomycin) for 24h cultured in suspension format in a humidified incubator with 5% CO2 atmosphere at 37° C. On experiment day, cells were harvested and re-suspended at a density of 1×10\hat{ }6/ml in assay buffer at a density of 1×10\hat{ }6/ml (1 part stimulation buffer (Cisbio): 5 parts ddH20) containing LiCl with 0.5% bovine serum albumin (BSA, Sigma).

Antibody stocks are serially diluted 1:2 over a 10-point concentration curve in order to determine an IC₅₀ value. Antibody lead clones were tested in challenge against SLIGKV (1 μM). A 5 μL aliquot of either concentration of antibody in replicate was added to a half area white 96-well plates (Corning) followed by 25 μL PAR2 of cell suspension. Plates were incubated at 37° C. for 30 minutes prior to addition of 5 μL/well SLIGKV for a final agonist challenge concentration of 1 μM. Plates were re-incubated for a further 30 minutes at 37° C. prior to addition of 10 μL IP-One detection kit (Cisbio) in lysis buffer. Cell plates were placed on a plate shaker at room temperature (RT) for 1 hour before reading plates on the PHERAstar FS microplate reader (BMG Labtech) using standard HTRF protocols, with excitation at 335 nm and emissions read at both 620 nm and 665 nm. HTRF ratios were calculated as in equation 1. Responses were normalised for % fold inhibition over 1 μM SLIGKV alone final assay concentration and results fitted in GraphPad Prism version 7.04 to a 4-parameter sigmoidal dose-response curve (Equation 2).

Table 10: Functional Characterization of Lead Clones Against SLIGKV that Demonstrate Antagonist Activity in Replicate in Cisbio IP-One Cynomolgus PAR2 or Rhesus PAR2 Receptor Assay.

| | | Cyno PAR2 SLIGKV | SLIGKV fold |
|---|---|---|---|
| Parent clone | Lead Clone | IC₅₀ (nM) ± SD | inhibition |
| Y022059 | Y022856 | >3162 | 0 |
| Y022065 | Y022858 | 300 | 2.1 |
| Y022065 | Y022870 | 25 | 2.2 |
| Y022065 | Y022877 | 37 | 2.3 |
| Y022065 | Y022879 | 63 | 2.2 |
| Y022065 | Y022882 | 96 | 2.2 |
| Y022065 | Y022883 | 25 | 2.4 |
| Y022065 | Y022884 | 385 | 2.1 |
| Y022065 | Y022885 | >3162 | 0 |
| Y022065 | Y022889 | >3162 | 0 |
| Y022065 | Y022881 | 41 | 2.0 |
| Y021171 | Y022916 | >3162 | 0 |
| Benchmark 1 | | >759 | 0 |
| Benchmark 2 | | NA | 0 |
| MAB3949 | | >1000 | 0 |
| | | Rhesus PAR2 | |
| | | SLIGKV IC₅₀ | SLIGKV fold |
| Parent clone | Lead Clone | (nM) ± SD | inhibition |
| Y022065 | — | >2000 | 0 |
| Y022065 | Y022870 | 51 | 1.9 |
| Y022065 | Y022877 | 47 | 2.0 |
| Y022065 | Y022883 | 43 | 1.8 |
| Benchmark 1 | | >398 | 0 |

### Functional Assay: Cell-Based IP-One Gag Assay at Human PAR1

Functional characterization was performed to test lead clone cross-species antagonist activity against human PAR1 receptor in challenge against synthetic agonist, SFLLR using the Cisbio® IP-One Gαq assay at two concentrations in replicate.

HEK293f cells were infected with a 5% v/v human PAR1 BacMam virus in the presence of 0.5 mM sodium butyrate in growth media (Pro293, 5% FBS, 1% Glutamax, 0.4% penicillin/streptomycin) for 24 hours cultured in suspension format in a humidified incubator with 5% CO2 atmosphere at 37° C. On experiment day, cells were harvested and re-suspended at a density of 1×10\hat{ }6/mL in assay buffer at a density of 1×10\hat{ }6/ml (1 part stimulation buffer (Cisbio): 5 parts ddH20) containing LiCl with 0.5% bovine serum albumin (BSA, Sigma).

Antibody stocks are serially diluted 1:2 over a 10-point concentration curve in order to determine an IC₅₀ value. Antibody lead clones were tested in challenge against SFLLR (100 nM). A 5 μl aliquot of either concentration of antibody in replicate was added to a half area white 96-well plates (Corning) followed by 25 μL PAR2 of cell suspension. Plates were incubated at 37° C. for 30 minutes prior to addition of Sul/well SFLLR for a final agonist challenge concentration of 100 nM. Plates were re-incubated for a further 30 minutes at 37° C. prior to addition of 10 μL IP-One detection kit (Cisbio) in lysis buffer. Cell plates were placed on a plate shaker at room temperature (RT) for 1 hour before reading plates on the PHERAstar FS microplate reader (BMG Labtech) using standard HTRF protocols, with excitation at 335 nm and emissions read at both 620 nm and 665 nm. HTRF ratios were calculated as in equation 1. Responses were normalised for % fold inhibition over 100 nM SFLLR alone final assay concentration and results fitted in GraphPad Prism version 7.04 to a 4-parameter sigmoidal dose-response curve (Equation 2).

As a positive antagonist control for the PAR1 human in vitro assay, a dose response curve of small molecule PAR1 antagonist Vorapaxar (Axon Medchem Cat 1755), tested from 10 μM final assay concentration in a 3-fold dilution series, was incubated and IC₅₀ determined.

### Table 11: Functional Characterization of Lead Clones Against SFLLR in Cisbio IP-One Human PAR1 Selectivity Assay

| | | Human PAR1 | SFLLR fold |
|---|---|---|---|
| Parent clone | Lead Clone | SFLLR IC₅₀ (nM) ± SD | inhibition |
| Y022059 | Y022856 | >3162 | 0 |
| Y022065 | Y022858 | >3162 | 0 |
| Y022065 | Y022870 | >3162 | 0 |
| Y022065 | Y022877 | >3162 | 0 |
| Y022065 | Y022879 | >3162 | 0 |
| Y022065 | Y022882 | >3162 | 0 |
| Y022065 | Y022883 | >3162 | 0 |
| Y022065 | Y022884 | >3162 | 0 |
| Y022065 | Y022885 | >3162 | 0 |
| Y022065 | Y022889 | >3162 | 0 |
| Y022065 | Y022881 | >3162 | 0 |
| Y021171 | Y022916 | >3162 | 0 |
| Benchmark 1 | | >759 | 0 |
| MAB3949 | | >1000 | 0 |
| Vorapaxar | | 51 | 2.5 |

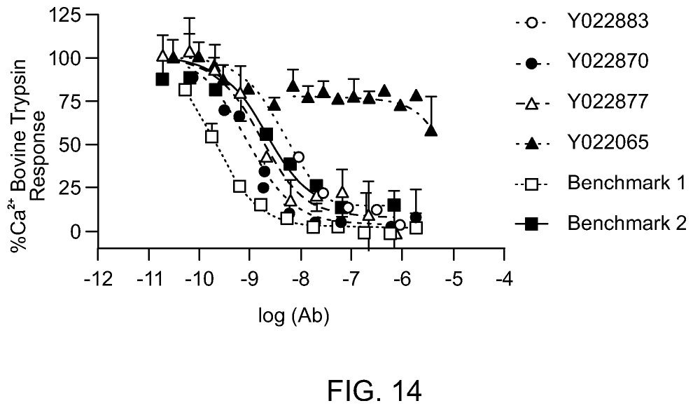

Lead Panel-Functional assay: cell-based FLIPR calcium mobilisation Gag assay using HT-29 cell line Following scale up, lead clones were characterised in HT-29 cells (following example 8 methodology) for confirmation of antagonist activity against 0.1 nM Bovine Trypsin challenge to endogenously expressed PAR2 (FIG. 14).

Beyond that, they displayed dual-activity in proteolytic cleavage within the extracellular N-terminus by trypsin, as well as inhibition of PAR2-AP activation. Analysis in the 3P specificity assay highlighted a few clones with an enhanced propensity to bind non-specific antigens and thereby reducing the number of dual-active IgGs for further consideration as therapeutic candidates. To refine the targeted epitope and for a preliminary epitope assessment, these IgG clones which also passed 3P assay QC were analysed in an ELISA-based epitope-binning experiment. All candidates were shown to compete with each other and probably target a proximal binding region different from the N terminal epitope of Benchmark 1. In depth characterization of the final candidate selection also included an affinity measurement by SPR.

The number of remaining clones was further reduced based on functional assays, sequence diversity and binding profile. These candidates were produced in a monovalent FabCys-AviH format and tested for cross-functionality to cynomolgus PAR2. In addition to that, affinity determination was refined with an optimized setup.

Y022870, Y022877 and Y022883 (three derivatives of Y022065) were selected as the final candidates and defined as the “lead panel” or “lead set” primarily based on performance in functional assays, SPR affinity data, production QC and non-specific binding to 32 different proteins in the protein panel profiling (3P) assay.

These three candidates were finally characterized in comparison to a set of six competitor antibodies. In depth characterization showed that the lead set binds to purified and cell surface expressed PAR2 with affinities within the range of the competitor antibodies. No off-target binding to related proteins PAR1, PAR3 and PAR4 could be detected. In contrast to all competitor (benchmark) antibodies, the lead set of antibodies provided herein is able to inhibit not only protease activation, but also peptide activation of PAR2, thus acting as dual inhibitors of PAR2 activation.

### Table 12: In Vitro Cross-Species Summary in SLIGKV Agonist Challenge

| Clone ID & IC50 (nM) | Y022065 | Y022870 | Y022877 | Y022883 |
|---|---|---|---|---|
| Human | 264 | 61 | 52 | 54 |
| Rhesus | >1995 | 50 | 47 | 43 |
| Cyno | >1995 | 88 | 74 | 68 |

### Example 9—Affinity and Kinetics Determination of Affinity Matured Antibody Clones

Determination of KD and k_{off} Using Label Free Methods for Biacore and Octet

For high throughput kinetic evaluation of IgGs, dissociation rate constants (k_{off}) were determined using either the Biacore T200 or Octet (QK384 or HTX) instrument. The same basic prerequisites, principles and considerations as described for full kinetic characterization apply, using monomeric antigen material (PAR2 StaR). For all 88 purified IgGs k_{off} was determined by Octet and showed a net improvement over the parental antibodies. kd [1/s] range from 1.1E-4 to 1.0E-05 assessed on truncated huPAR2 StaR and FL-huPAR2 StaR.

In general, only one analyte concentration was used for k_{off} determination. Samples that were assessed directly from IgG supernatants or bacterial lysates (Fab fragments), were subjected to k_{off} determination using Octet in an antibody capture format. As a prerequisite, a monomeric antigen was used as analyte. IgG samples were captured at a moderate capture level (0.4 nm) onto streptavidin sensors loaded with a high density of capture ligand (biotinylated MabSelect SuRe ligand). The monomeric antigen proteins human PAR2-1 variant (P151 55-377, truncated) and human PAR2-2 variant (P166: 1-377, full length) were used as analytes in solution. Dissociation was monitored for up to 1500 s. The recorded sensorgrams were fitted to a 1:1 binding model using the corresponding instrument-specific evaluation software (Biacore T200 Evaluation Software 2.x or 3.x|Octet Data Analysis 9.x or 10.x).

For all eleven IgG1f_AEASS, affinities to human PAR2 were determined by SPR. The IgGs were captured via their Fc-fragment and detergent (LMNG/CHS) solubilised FL-N PAR2 StaR was used as analyte in solution. 50 mM HEPES pH 7.5, 150 mM NaCl, 0.02% LMNG was used as assay buffer. To minimize artefacts introduced by non-specific interaction of FL-N PAR2 StaR with the sensors, the CM3 sensors were used as basis to immobilize a high density of anti-hu-Fc capture antibody (BR-1008-39, GE Healthcare). IgGs were captured with relatively high levels of approximately 500 RU to achieve a higher ratio of specific to non-specific binding (while still maintaining conditions suitable for kinetic characterization). Affinities ranged from 6.5 nM to 130 pM. The k_{off} values of the best candidates were at the lower assay limit. A minimum k_{off} of 2.00E-05 s⁻¹ was reported (corresponding to 3% dissociation in 1500 s). A range of affinities were observed with KD values from 6.5 nM to 130 pM and the majority demonstrated an improvement on the parental clones Y022065 (K_{D}=5.3 nM) following the affinity maturation process, as would be expected.

### Table 13: SPR Kinetics of Affinity Matured Clones as Determined Using Octet System and Soluble PAR2 StaR®

| Clone | kₐ (1/Ms) | K_{d} (1/s) | K_{D} (nM) |
|---|---|---|---|
| Y022916 | 3.9E+4 | 2.5E−4 | 6.5 |
| Y022856 | 1.2E+5 | 2.5E−4 | 2.5 |
| Y022858 | 5.2E+4 | 7.3E−5 | 1.4 |
| Y022870 | 9.6E+4 | 2.0E−5 | 0.21 |
| Y022877 | 6.8E+4 | 2.0E−5 | 0.29 |
| Y022879 | 5.9E+4 | 3.2E−5 | 0.5 |
| Y022882 | 1.1E+5 | 2.0E−5 | 0.18 |
| Y022883 | 1.4E+5 | 2.0E−5 | 0.14 |
| Y022884 | 7.1E+4 | 8.4E−5 | 1.2 |
| Y022885 | 1.5E+5 | 2.0E−5 | 0.14 |
| Y022889 | 1.6E+5 | 2.0E−5 | 0.13 |
| Benchmark1 | 1.2E+6 | 8.8E−4 | 0.76 |

### Table 14: SPR Kinetics as Determined Using Biacore System and Soluble PAR2 StaR® and Analysis of the Top Three Affinity Matured Clones of Interest

| Clone | ka (1/Ms) | kd (1/s) | KD (M) |
|---|---|---|---|
| 22870 | 3.4E+04 | 7.0E−06 | 2.1E−10 |
| 22883 | 3.0E+04 | 1.2E−05 | 4.0E−10 |
| 22877 | 2.7E+04 | 8.0E−06 | 3.0E−10 |

Table 15: SPR Kinetics as Determine Using Biacore System Using Human PAR2 StaR® Incorporated into Nanodiscs-Comparison of Three Affinity-Matured Clones with a Non-Affinity Matured Clone (Y021171).

| Clone | ka (1/Ms) | kd (1/s) | KD (M) |
|---|---|---|---|
| Y021171 | 1.2E+06 | 3.7E−02 | 3.1E−08 |
| Y022870 | 9.3E+04 | 2.5E−04 | 2.7E−09 |
| Y022877 | 7.3E+04 | 2.1E−04 | 2.8E−09 |
| Y022881 | 1.3E+05 | 8.3E−04 | 6.3E−09 |

SPR Affinity Measurements Using Recombinant PAR2 StaR® Protein and Evaluation of the Impact of Temperature on Binding: Human PAR2 Binding to Immobilised mAbs at 25° C. And 37° C.

The assay of PAR2 interaction with anti-PAR2 antibodies was carried out using a Biacore T200 instrument (GE Healthcare). Anti-human IgG antibody (Human Antibody Capture Kit, GE Healthcare) was immobilised by amine coupling in the target and reference flow cells of sensor chip CM3 (GE Healthcare) to a surface density of 2700 to 3000 resonance units (RU). Immobilisation was carried out at 25° C. in HBS-EP+buffer (GE Healthcare). The buffer was then changed to 50 mM HEPES, pH 7.5, 150 mM NaCl, 0.02% LMNG, 0.002% CHS for the PAR2-antibody interaction assay which was carried out at either 25 or 37° C. Each cycle of the assay began with antibody capture in the target flow cell to a level of 400 to 450 RU. PAR2 was then injected over target and reference flow cells at three concentrations, 5 min each, followed by 30-min dissociation. The surface was then regenerated with a solution provided in the kit (Human Antibody Capture Kit, GE Healthcare). With every antibody tested the first cycle was a blank (three injections of the running buffer) and the second a series of 50, 100, and 200 nM PAR2 injections. The blank-subtracted data were fitted to 1:1 interaction model to obtain association and dissociation rate constants, ka and ka, and the affinity constant K_{D}.

Table 16: Summary of Binding of Lead Clones after Affinity Maturation at 25° C. And 37° C.

| | | Dissociation | | | | |
|---|---|---|---|---|---|---|
| | Temp | time | ka | kd | KD | t½ |
| Clone | ° C. | (mins) | (1/Ms) | (1/s) | (M) | (h) |
| Y22870 | 25 | 90 | 3.4E+04 | 7.0E−06 | 2.1E−10 | 27.6 |
| Y22870 | 25 | 30 | 2.8E+04 | 1.2E−05 | 4.3E−10 | 16.1 |
| Y22870 | 37 | 30 | 4.1E+04 | 1.9E−05 | 4.5E−10 | 10.3 |
| Y22877 | 25 | 90 | 2.7E+04 | 8.0E−06 | 3.0E−10 | 24.1 |
| Y22877 | 25 | 30 | 2.2E+04 | 5.1E−06 | 2.3E−10 | 37.9 |
| Y22877 | 37 | 30 | 3.1E+04 | 1.7E−05 | 5.5E−10 | 11.5 |
| Y22883 | 25 | 90 | 3.0E+04 | 1.2E−05 | 4.0E−10 | 16.2 |
| Y22883 | 25 | 30 | 4.0E+04 | 8.1E−06 | 2.0E−10 | 23.7 |
| Y22883 | 37 | 30 | 3.6E+04 | 2.3E−05 | 6.5E−10 | 8.3 |

### Assessment to Determine if a Lead Clone Exhibits pH Sensitive Binding

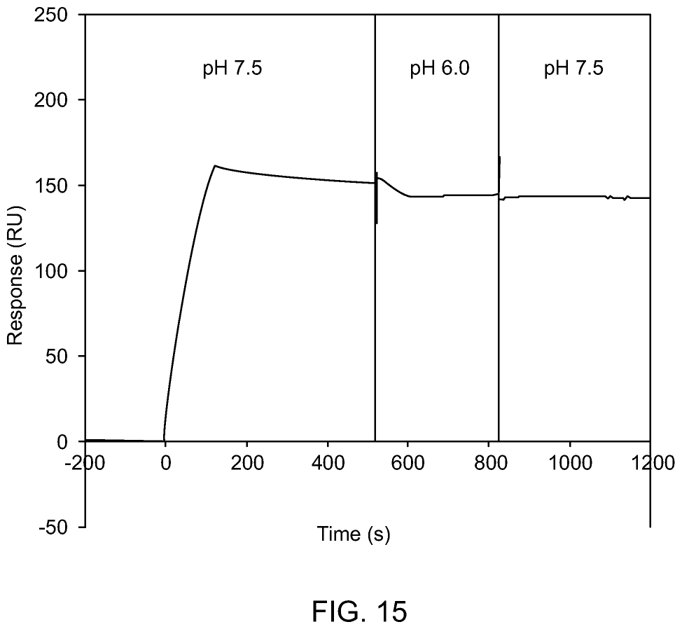

Y22883 was captured to 280-320 RU and 100 nM PAR2 was injected for 2 minutes. About 400 seconds after injection stop a pH 6.0 buffer was injected for 5 minutes to see if it could cause a faster dissociation. The experiment was designed is such a way that numerical evaluation of dissociation rate constants is not possible. Nevertheless, it is clear that the conditions at pH 6.0 do not accelerate dissociation (FIG. 15).

### Apparent K_{D} Determination on Whole Cells by KinExA

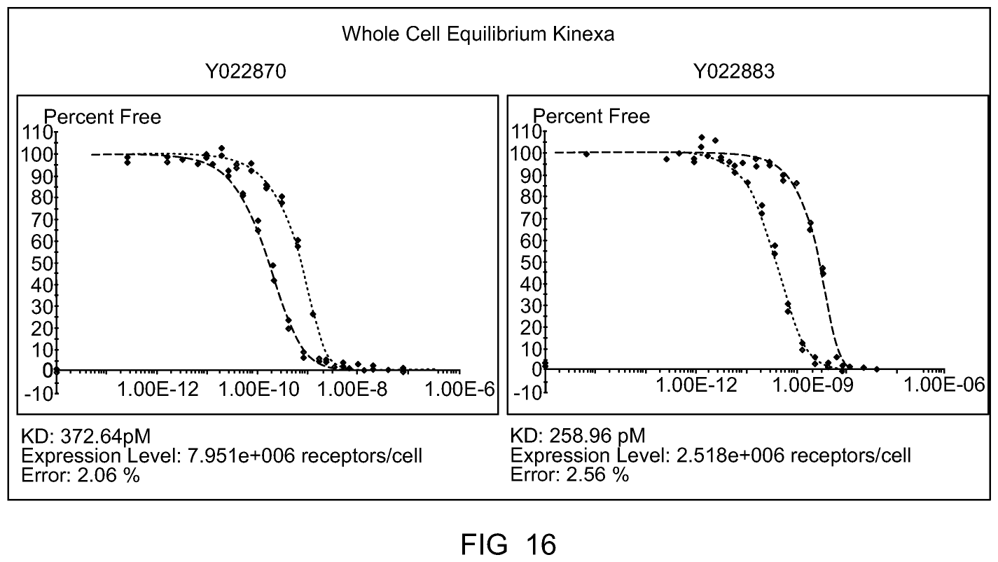

For the determination of apparent K_{D} using “native” PAR2 (i.e. PAR2 expressed at the cell surface) KinExA methodology was applied to evaluate a subset of lead clones, namely Y022870 and Y02283. To determine the equilibrium KD to surface-expressed antigens, instead of soluble antigen, BacMam infected HEK-293F cells, expressing the wild-type full-length human PAR2, were used for titration. The cells were titrated in incubation buffer (D-PBS from Gibco, 0.5% (w/v) BSA, 0.02% (v/v) Sodium azide). The experiment consisted of two titrations with a low and a high fixed IgG concentration. In both titration experiments (stoichiometry- and affinity-controlled conditions), the maximum amount of cells had to contain a high enough effective ligand concentration to completely saturate the IgG. The mixtures of IgG and cells were incubated at room temperature overnight under very gentle agitation to allow for equilibrium formation. Subsequently, the formed cell-antibody complex was removed by centrifugation. The free IgG amounts of the supernatants were determined with goat anti-human F(ab′)₂ fragment specific antibody (Jackson ImmunoResearch, #109-005-097) coated on PMMA (Polymethylmethacrylat) beads and fluorescence detection with anti-human F(ab′)₂ fragment specific Alexa Fluor 647 antibody (Jackson ImmunoResearch, #109-605-097). As IgG molecules were used in the KinExA experiment, the resulting values are labelled ‘apparent KD’, to point out that bivalent binding on the antigen presenting cells cannot be excluded, and binding may be reinforced by contributions of avidity. The recorded titration curves were analyzed with KinExA Pro software 4.1.11 using the n-curve analyse tool by applying the “Equilibrium, whole cell” model. Y022870 binds to wild type human PAR2 expressed on whole cells with an affinity of ˜370 pM, whereas Y022883 has an affinity of ˜260 pM (FIG. 16). Values are in line with SPR determined using PAR2 nanodiscs (210 and 140 pM, respectively).

### Example 10—Epitope Binning and Analysis of Affinity Matured Clones by HDX and Flow Cytometry Analysis of Receptor Mutations

### Elisa Based Epitope Binning by Antibody Competition

Epitope binning was performed by an ELISA-based assay. Anti-human PAR2 IgG1f sample IgGs were directly coated on a microtiter plate at a concentration of 100 nM. In parallel a tagged antigen (e.g. nanodisc-embedded StaR) at a constant concentration (12.5 nM to 25 nM) was incubated with a 25-fold molar excess of a second anti-human PAR2 IgG1f to saturate all epitopes (maximum conc. 312.5 nM to 625 nM, 7-point titration, 1:4) for 1 hour and complexes were allowed to form. An internal positive control (self-competition) was included for each sample IgG assessed. Antigen-IgG complexes were added to IgG coated plates for 30 min. Antigen-IgG complexes bound to coated IgGs were detected via a tag-specific antibody (e.g. anti-StrepII or anti-His) detected with a suitable alkaline-phosphatase (AP) coupled secondary antibody in combination with ‘AttoPhos’ fluorescence substrate. Multiple washing steps were performed in between individual assay steps. In the event of competition for a same epitope, antigen-IgG complexes cannot bind to IgG coated plates which translates into low signals. Targeting of different epitopes allows antigen-IgG complexes to bind coated IgGs and IgGs in solution simultaneously, leading to positive detection signals.

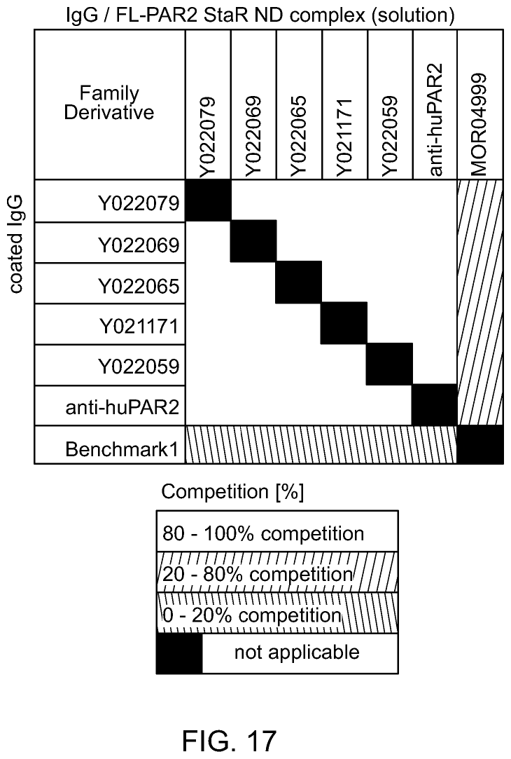

This competition ELISA set-up was employed to characterise the targeted epitope and epitope diversity of the available candidates by epitope binning. The 16 dual-functional PAR2-specific candidates were tested for binding competition against each other and against reference antibodies, including MAB3949 (R&D Systems) and Benchmark 1. This set of 16 antibodies included at least one derivative of each of the five parental families i.e., clones derived from the parental clones by affinity maturation (Y022079, Y022069, Y022065, Y021171, Y022059). All affinity matured families competed with each other, as well as anti-huPAR2 (MAB3949 R&D Systems). This indicates that all these antibodies target proximal binding regions (i.e., extracellular domains other than the N terminus). None of the candidates competed with Benchmark mAb 1. Benchmark mAb 1 is the only antibody in the panel tested that exclusively binds to full length PAR2, i.e., binds a linear N-terminal epitope; these results indicate that affinity matured candidates bind to a distinctly different epitope that is more closely related to or directly targeting the extracellular loop region (FIG. 17).

In addition, assessment of binding under StaR-denaturing conditions was implemented to aid categorization into recognition of linear or conformational epitope. 97% of tested IgG candidates displayed loss of binding and were therefore assigned as binding to a conformational epitope.

### Hydrogen Deuterium Exchange

Hydrogen Deuterium Exchange (HDX) is one of the methods that can be used to interrogate and identify a binding interface. Protein-protein interaction sites are probed by cataloguing the hydrogen/deuterium exchange rate of the amide hydrogens in proteins backbone. How fast or slow this exchange takes place is determined by the accessibility of those amide hydrogens to the solvent. Therefore, amides in exposed regions should have a higher rate of exchange than those buried within a protein-protein interface since they have a higher accessibility to the solvent environment.

The following equipment and materials were used in the example. Liquid chromatography separation was performed on Thermo Vanquish UHPLC (Thermo Scientific) equipped with two temperature-controlled column compartments. Online digest was performed on Enzymate BEH Pepsin column 2.1×50 mm (Waters) at 15° C. and obtained peptides were separated on Kinetex Evo C18 2.6 μm 50×2.1 mm (Phenomenex) at 4° C. Mobile phase for separation of peptides consisted of acetonitrile/0.1% formic acid (FA) and water/0.1% FA (Fisher Chemical). Separation was achieved using 17.5 minutes gradient from 0% to 40% of acetonitrile/0.1% FA. MS1 and MS/MS analysis of obtained peptides was performed on Orbitrap Fusion instrument (Thermo Scientific). Peptide analysis was done using Proteome Discoverer 2.4 with Byonic node (Thermo Scientific) and HDX processing was performed using HDExaminer 2.4 (Sierra Analytics). Genetically modified PAR2-2 1-377 (glycosylation sites removed) PAR2 StaR protein was purified in LMNG/CHS. The antigen-binding fragment (Fab) Y022883 was directly expressed (see Example 4) and reference Fab was derived from papain digest of R&D IgG MAB3949.

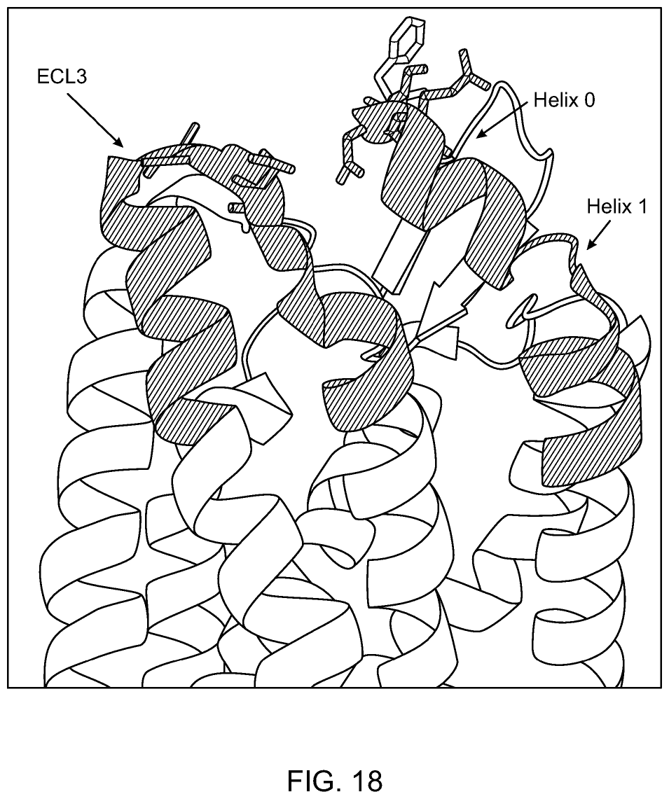

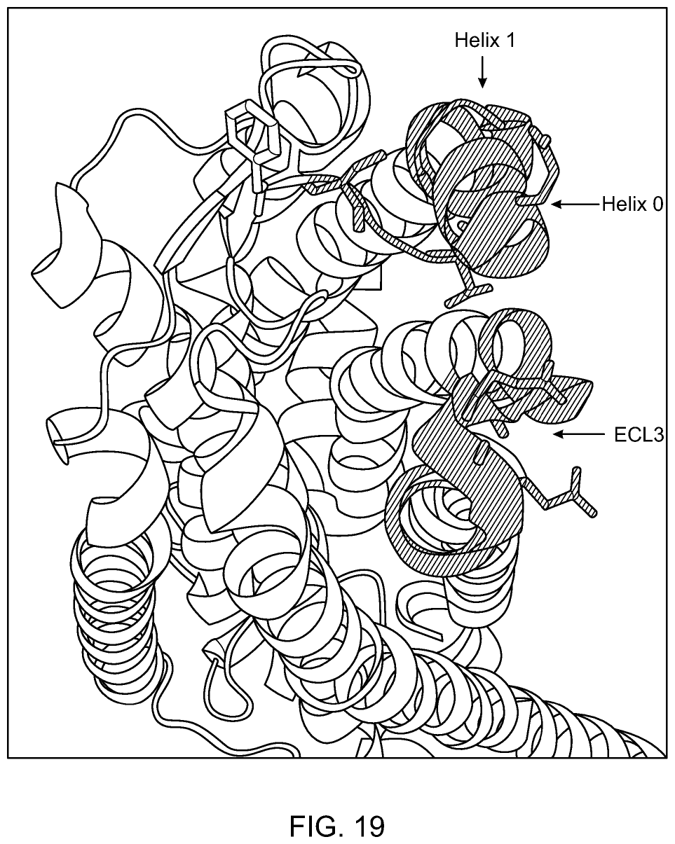

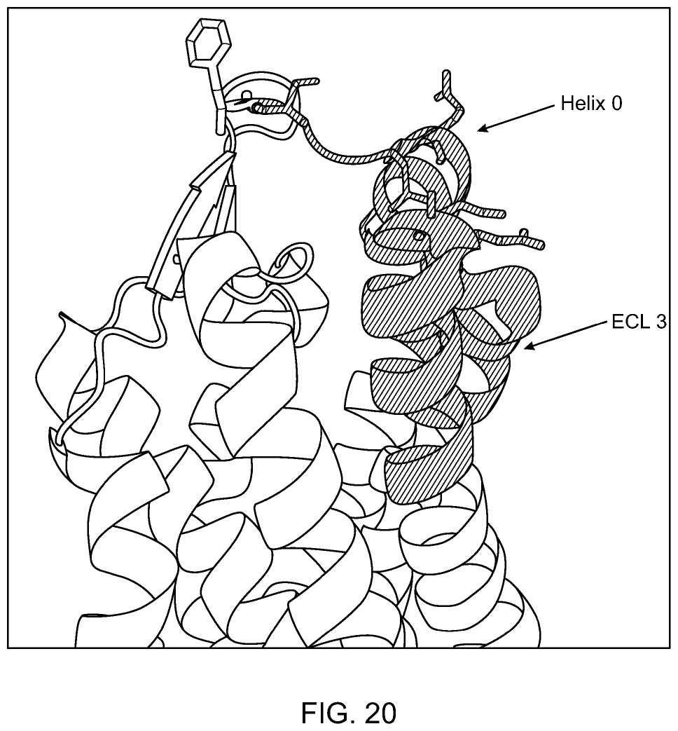

A typical bottom-up HDX mass spectrometry (MS) procedure for HDX comprises protein-Fab incubation, isotope labelling with deuterated buffer, quench, proteolytic digestion, desalting/separation, and MS analysis. PAR2-2 construct was diluted to 50 μM and incubated with equimolar concentration of Fab Y022883 (or Fab MAB3949) for 30 minutes at 22° C. HDX was initiated by dilution of protein mixture in a deuterated buffer (10 mM phosphate buffer pH 7.5, 150 mM NaCl) for each of several periods of time (continuous labelling): 1 min, 10 min, 30 min, 2 h at 22° C. The dilution of 20-fold was chosen to achieve up to 90-95% D labelling of solvent accessible hydrogens (usually 2 μl of protein mixture was mixed with 38 μl of deuterated buffer). After desired time, the exchange was quenched by lowering the solution pH to ˜2.5 with 0.8% formic acid and solution was immediately snap frozen on dry ice to reduce back-exchange. Samples were then defrosted before enzymatic on-line digestion on pepsin column and subsequent analysis on LC-MS. The same protocol was then followed for PAR2-2 construct only and Fab Y022883 (or Fab MAB3949) only in order to identify solvent/deuterium-accessible hydrogens in non-complexed proteins. Comparison of deuteration levels in the PAR2-2/Fab Y022883 (or PAR2-2/Fab MAB3949) complex with the deuteration levels of homogeneous proteins (either Fab Y022883, Fab MAB3949 or PAR2-2) gives indication of changes to the surrounding of the solvent accessible hydrogens and subsequently such “mapping” could be used to determine PAR2-2/Fab Y022883 (or PAR2-2/Fab MAB3949) interaction sites. In order to validate the methodology, the HDX results obtained for PAR2/MAB3949 Fab were mapped on the published co-structure of PAR2 in complex with the MAB3949 Fab (Cheng et al., Nature, 2017). The interaction deduced from the HDX matched well the one observed in the crystal co-structure. The PAR2-2/Fab Y022883 HDX was then interrogated and data indicated a different interaction (FIGS. 18-20) to the one observed for Fab MAB3949 (Cheng et al., Nature, 2017). The N-terminal region of PAR2 showed a distinctive deuterium-hydrogen exchange pattern which likely suggests involvement of this region in binding.

Table 17: List of PAR2-2 Peptides, Identified by HDX, which are Determined to be Located in the Interacting Region and Therefore Comprise the Y022883 Antibody Epitope.

| Peptide | Location in PAR2-2 |
|---|---|
| SEQ ID NO: 41-55-59 VETVF | Segment1 |
| SEQ ID NO: 42-60-77 SVDEFS ASVLTGKLTTVF | Segment1/Helix0/1 |
| SEQ ID NO: 43-306-311 LLVVHY | ECL3 |
| SEQ ID NO: 44-312-326 FLIK SQGQSHVYALY | ECL3 |

These HDX data suggest that ECL1 or ECL2 of PAR2 are much less likely to be involved in direct interactions with the Y022883 PAR2 antibody; the direct interactions observed using this methodology are located in the region of Segment1, Helix0/1 (proximal to the trans-membrane domain and in contrast to other PAR2 targeting antibodies that bind to relatively distal regions of the receptor) and ECL3.

### Mutational Analysis Using Flow Cytometry

PAR2 binding epitope mutations were evaluated by flow cytometry on human PAR2 expressing HEK293F-cells as follows. Monitoring of expression levels was undertaken using Benchmark 1 antibody. Mutations introduced in the WT PAR2 background were targeting either Segment1, Helix0/1 (single mutations F59A, S60W, D62G, D62F, E63K, E63A and a double mutation D62G/E63G) or ECL3 (single mutations Q317A, G318F, Q319A, Q3191). The various versions of PAR2 were introduced into the HEK293F cells by BacMam infection.

200 UL HEK293F-cells at 4×10\hat{ }6 cells/mL in FACS-buffer (PBS (Sigma, #F9665), 1% BSA (Sigma, #A9647) and Roche Complete protease inhibitors (#11836145001)) were agitated with either Benchmark 1 or Y022883 at 20 nM for 1 hour at 4° C. Cells were pelleted and washed three times with 200 μL FACS buffer. Successively, the cells were agitated with 200 μL secondary allophycocyanin (APC) conjugated anti-human IgG antibody at 20 nM for 1 hour at 4° C. and washed again three times with 200 μL FACS buffer.

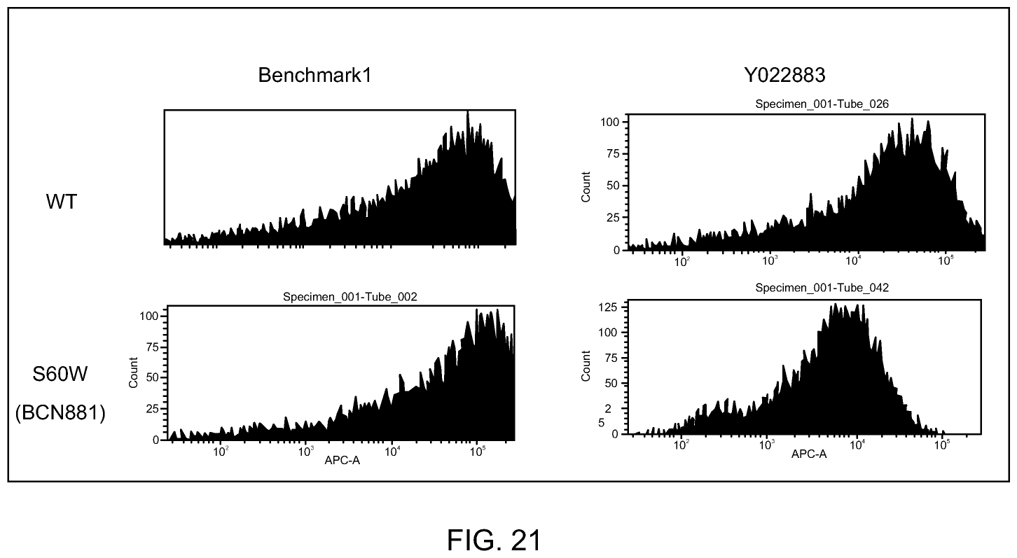

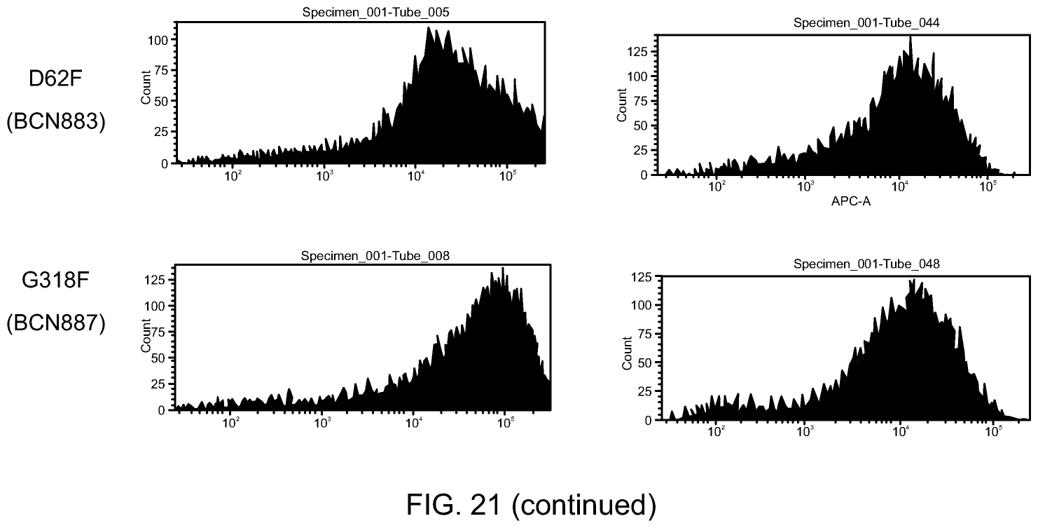

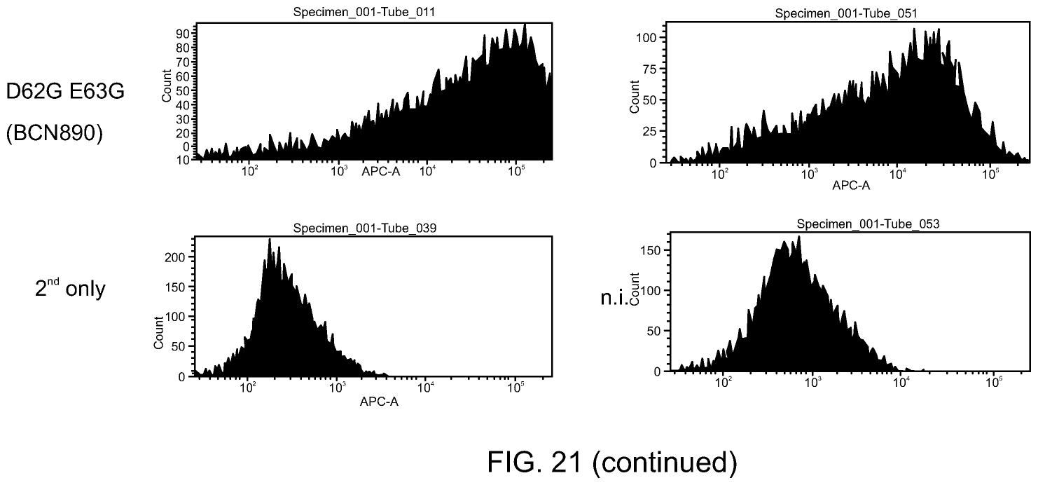

Mutated PAR2 and WT PAR2 showed very similar expression profiles when detected with Benchmark 1, except for D62F, which shows a partially reduced expression level, whereas S60W, D62F, G318F and the double mutation D62G/E63G all show a reduced expression profile versus WT when detected with Y022883, indicating that the areas identified by HDX analysis are involved in Y022883 binding to human PAR2. The observed reductions in binding, with the chosen mutations, are stronger for Segment1/Helix0/1 than ECL3 (FIG. 21).

The impact of these mutations may confer a conformational change that alters the antibody epitope and may also cause a change in functional activity of the receptor.

### Example 11—Rat Pharmacokinetics

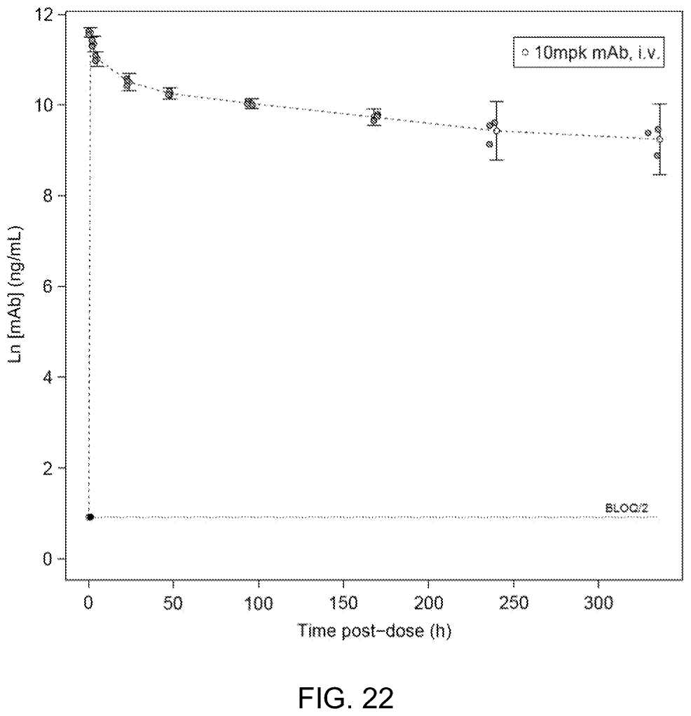

The pharmacokinetics of Y022883 were explored in adult male Sprague Dawley rats. 3 rats were intravenously injected with 10 mg/kg Y022883 and blood samples taken at various timepoints out to 2 weeks. Blood samples were used to generate sera and these sera analysed by a qualified non-GLP ELISA method on the Gyrolab platform (generic PK kit) with PK parameters calculated by non-compartmental analysis (NCA) using Phoenix™ WinNonlin. Summary data are shown in FIG. 22 and Table 18. These data are consistent with expectations of a human mAb that does not have a significant antigen sink which would be expected as Y022883 does not bind rat PAR2.

Table 18: Group PK Parameters of Y022883 in Rats (n=3). Data are Shown as Mean Average with Standard Deviation in Brackets.

| | Half-life | Cmax | AUCₐₗₗ |
|---|---|---|---|
| Group | (h) | (ng · mL⁻¹) | (hr · ng · mL⁻¹) |
| 10 mg/kg Y022883 | 146.7 | 109136 | 6937883 |
| | (15.0) | (4502) | (496973) |

### Example 12—Cynomolgus Pharmacokinetics and Pharmacodynamics

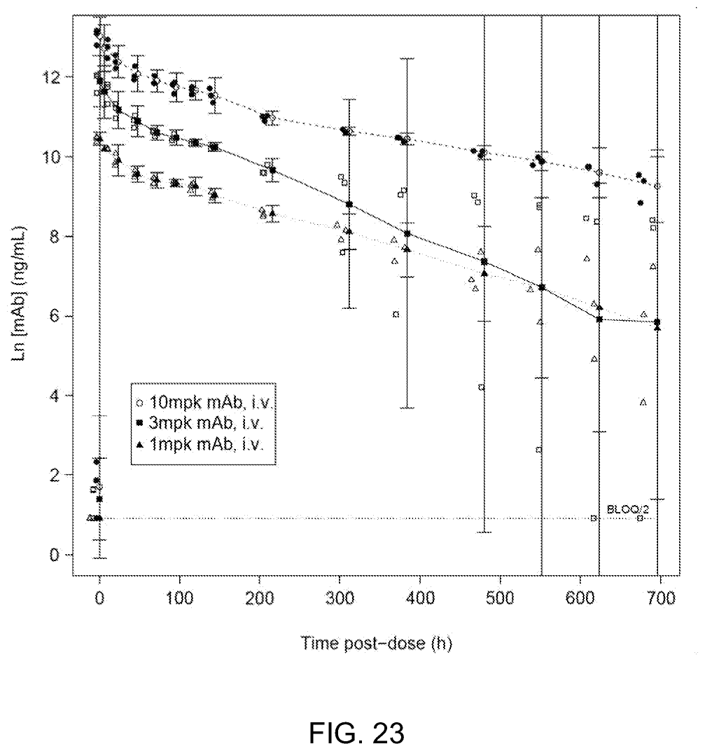

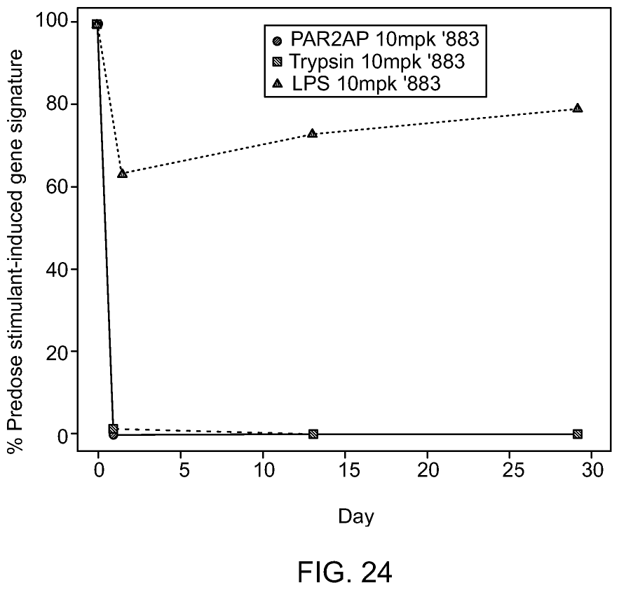

The pharmacokinetics of Y022883 were explored in adult male cynomolgus monkeys. 3 groups of 3 male, adult cynomolgus monkeys were intravenously injected with 10 mg/kg, 3 mg/kg, or 1 mg/kg Y022883 and blood samples taken at various timepoints out to 4 weeks. Blood samples were used to assess pharmacodynamics (FIGS. 24-26, inclusive) or to generate sera for pharmacokinetics (see FIG. 23).

Sera were analysed by a qualified non-GLP ELISA method on the Gyrolab platform (generic PK kit) with PK parameters calculated by non-compartmental analysis (NCA) using Phoenix™ WinNonlin. Summary data are shown in FIG. 23 and Table 19. These data are consistent with expectations of a human mAb that does not have a significant antigen sink.

Table 19: Group PK Parameters of Y022883 in Cynomolgus Monkeys (n=3). Data are Shown as Mean Average with Standard Deviation in Brackets.

| Group | Half-life (h) | | Cmax (ng · mL⁻¹) | | AUCₐₗₗ (hr · ng · mL⁻¹) | |
|---|---|---|---|---|---|---|
| 10 mg/kg Y022883 | 182.3 | (66.3) | 463117 | (85204) | 45635218 | (5182962) |
| 3 mg/kg Y022883 | 174.5 | (132.5) | 150540 | (35878) | 12188113 | (2805691) |
| 1 mg/kg Y022883 | 150.8 | (97.3) | 34523 | (2255) | 3588673 | (168372) |

Pharmacodynamics were assessed under sterile conditions at 4 timepoints (pre-dose, 1, 13 and 29 days post-dose). 1 volume of PBS was added to each blood sample and 6% dextran T-500 added to a final concentration of 2%. After a 25-40 minute incubation (reaction stopped when the 2 phases of leukocyte-rich and erythrocyte-rich were 50% each) the leukocyte-bearing supernatant was removed, washed in PBS and re-suspended in culture media (RPMI-1640, 2% FCS, 1% penicillin-streptomycin, 1% L-glutamine, 1% non-essential amino acids) at original blood volume. 1 mL cell samples were then challenged with PBS, 100 mcM 2f-LIGRLO, 100 nM trypsin or 100 pg/mL LPS and incubated at 37° C. for 6 hours. Samples were then pelleted (306 g, 5 minutes, room temperature), the pellet re-suspended and mixed with 0.48 mL RNAlater before being frozen at −70° C. Sample RNA was then extracted using standard methods and the transcriptome assessed with genome-based read mapping at a read depth of 50×.

Sequencing reads (FASTQ files) were aligned to a reference genome and the number of reads mapped to each gene counted which in turn generated a gene-count table. Differential gene expression analysis was then performed between each stimulus at each timepoint per dose group e.g. 1 mg/kg predose PBS vs 1 mg/kg predose PAR2AP, 1 mg/kg predose PBS vs 1 mg/kg predose trypsin, 1 mg/kg predose PBS vs 1 mg/kg predose LPS. Differential gene expression was then determined via the generation of volcano plots (scatterplot of fold change in gene expression vs statistical significance (P value)). Genes that were greater-than-or-equal to 2-fold up- or down-regulated and had a significance of less than or equal to 0.05 were considered differentially expressed. Hence a set of differentially expressed genes was generated for each stimulus (from the pre-dose samples); the effect of treatment with mAb was evaluated by determining whether the set of pre-dose differentially expressed genes for each stimulus were still differentially expressed at each timepoint. For example, 1816 genes were differentially regulated by LPS (vs predose PBS) at the predose timepoint in the 1 mg/kg dose group; at 24h 1379 of these genes were still differentially regulated by LPS (vs 24h PBS) hence 76% of the predose LPS gene-signature was present at 24h post-dose in the 1 mg/kg LPS dose group.

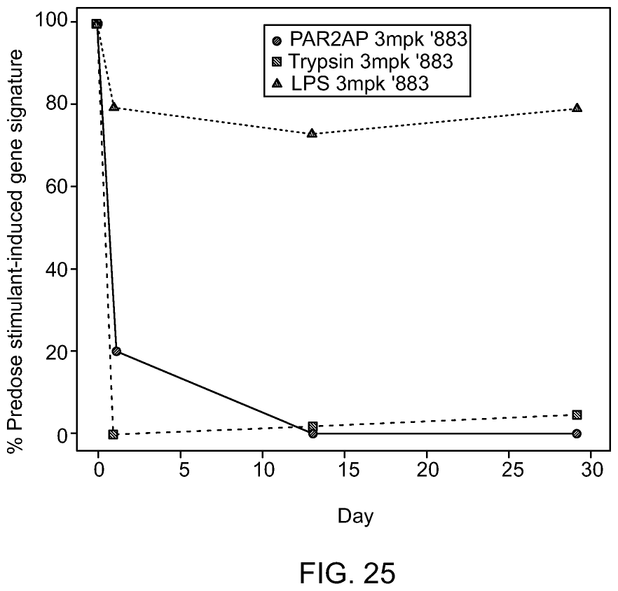

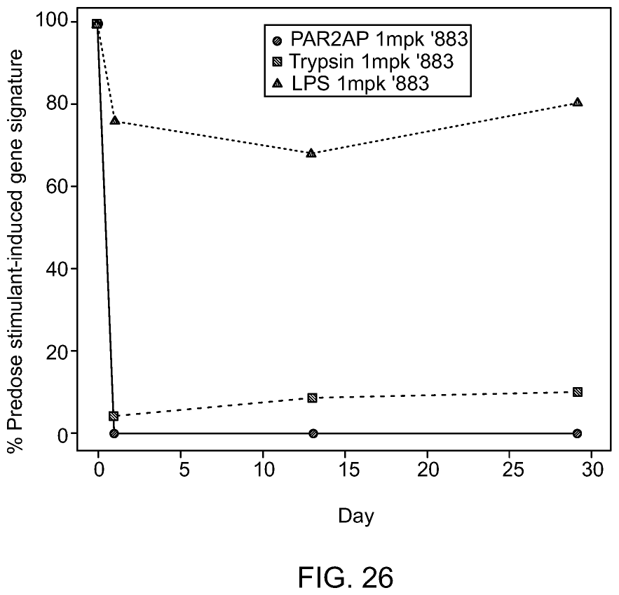

Using this methodology a single IV infusion of 10 mg/kg Y022883 was found to suppress the PAR2AP-induced and the trypsin-induced gene signature at all timepoints whereas the LPS gene signature was suppressed by 36% at 24 hours before recovering to 23% suppression by 29 days (FIG. 24). An analogous set of observations was made following 3 mg/kg Y022883 (FIG. 25) although with 1 mg/kg Y022883 the trypsin gene signature had started to recover by day 29 (96%, 92%, 91% suppression at 24h, day 13, day 29; FIG. 26). These data are consistent with Y022883 specifically antagonising PAR2 (versus at least TLR4) and providing blockade of PAR2 on peripheral blood leukocytes for a month following a single infusion of at least 1 mg/kg. The partial suppression of the LPS-induced gene signature by PAR2 antagonism is consistent with literature reports linking the TLR4 and PAR2 signalling cascades (Yamaguchi et al 2016).

### Example 13 Effects of SH-C and Benchmark II on Trypsin and Par2-AP Induced p38 MAPK and PERK Phosphorylation in T84 Cell Line

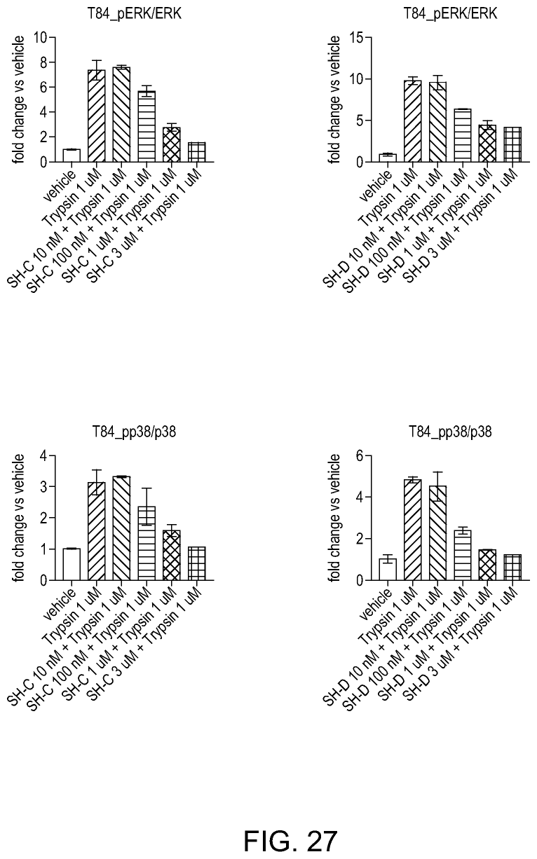

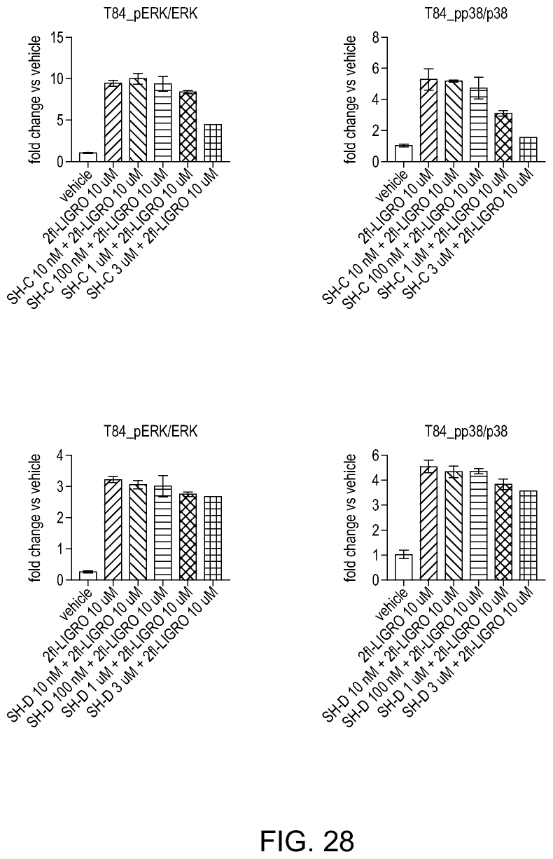

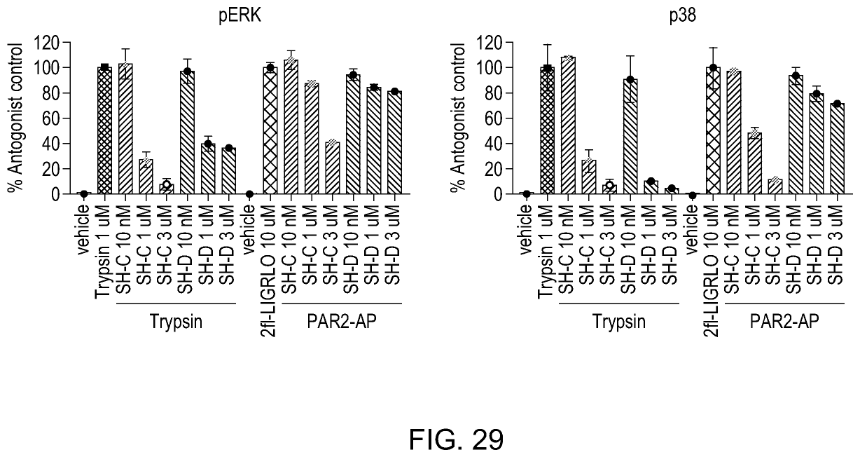

T84 cells were treated with Trypsin (1 μM) or PAR2-AP (2fu-LIGRLO) (10 μM) in growth medium. Antibodies Y022883 (SH-C) and Benchmark 2 (SH-D) were tested at concentrations ranging from 10 nM to 3 μM. Cells were lysed at 5- or 30-min post treatment in lysis buffer (PBS+1% Triton X-100, protease and phosphatase inhibitors). Total protein concentration was evaluated by BCA before phosphoprotein analysis. Protein concentration was adjusted to 0.3 mg/ml and samples were loaded onto a 12-230 kDa Wes Separation Module 8×25 capillary cartridges, Protein Simple. Rabbit Antibodies were used to detect pERK and ERK (PERK, Cell signalling, cat: 9101S; ERK, Cell signalling, cat: 4695S) and phospho p38 MAPK and p38 MAPK (Cell signalling, cat: 9211L; p38, Cell signalling, cat: 9212S). Anti-rabbit detection module (Protein Simple, cat: DM-001) was used as the secondary antibody. Data for phospho-ERK or phospho p38 was normalized to data for total ERK or total p38; biological duplicates were analyzed. Percentage of the maximum ratio was used to normalized between plates (FIGS. 27-29).

Treatment with Trypsin or PAP2-AP produced a >8 fold increase in PERK and a >3 fold increase in phospho p38-MAPK. Both SH-C and Benckmark II demonstrated inhibition of phosphorylation of ERK and p38-MAPK in trypsin treated cells. Only SH-C demonstrated significant inhibition of phosphorylation in PAR2-AP treated cells.

### Sequences

Tables 20-23: Antibody clone sequences for clones Y022065, Y022870, Y022877 and Y022883 with CDRs identified according to Kabat.

Variable Region Pair Name
Y022065

| | |
|---|---|
| Framework Vh | YLAN_VH3-23 |
| Framework Vl | YLAN_VL2-23 |
| HCDR1 (SEQ ID NO: 3) | SYAMS |
| HCDR2 (SEQ ID NO: 4) | AISGSGGSTYYADSVKG |
| HCDR3 (SEQ ID NO: 5) | GFHYTHSGKRYYYPFDI |
| LCDR1 (SEQ ID NO: 7) | TGTSSDVGSYNLVS |
| LCDR2 (SEQ ID NO: 8) | EGSKRPS |
| LCDR3 (SEQ ID NO: 9) | SQYATFIAFAV |
| Protein Sequence (VH) | EVQLLESGGGLVQPGGSLRLSCAASGFTFSSYAMSWVRQAPGKGLEWVSAISGSGGST |
| (SEQ ID NO: 2) | YYADSVKGRFTISRDNSKNTLYLQMNSLRAEDTAVYYCARGFHYTHSGKRYYYPFDIWG |
| | QGTLVTVSS |
| Protein Sequence (VL) | QSALTQPASVSGSPGQSITISCTGTSSDVGSYNLVSWYQQHPGKAPKLMIYEGSKRPSG |
| (SEQ ID NO: 6) | VSNRFSGSKSGNTASLTISGLQAEDEADYYCSQYATFIAFAVFGGGTKLTVLGQ |
| Ppt Format | h_IgG1f_AEASS |
| LC Protein (SEQ ID NO: 70) | QSALTQPASVSGSPGQSITISCTGTSSDVGSYNLVSWYQQHPGKAPKLMIYEGSKRPSG |
| | VSNRFSGSKSGNTASLTISGLQAEDEADYYCSQYATFIAFAVFGGGTKLTVLGQPKAAPS |
| | VTLFPPSSEELQANKATLVCLISDFYPGAVTVAWKADSSPVKAGVETTTPSKQSNNKYA |
| | ASSYLSLTPEQWKSHRSYSCQVTHEGSTVEKTVAPTECS |
| LC DNA (SEQ ID NO: 34) | CAGAGCGCCC TGACCCAGCC AGCCAGCGTT AGCGGTAGCC CAGGCCAGAG CATTACCATT |
| | AGCTGCACCG GCACCAGCAG CGACGTGGGC AGCTATAACC TGGTTAGCTG GTATCAGCAG |
| | CATCCGGGCA AAGCCCCGAA ACTGATGATC TATGAAGGCA GCAAACGCCC GAGCGGCGTT |
| | AGCAACCGCT TTAGTGGCAG CAAAAGCGGC AACACCGCCA GCCTGACCAT TAGCGGCCTG |
| | CAAGCCGAAG ACGAAGCCGA TTATTACTGC TCCCAGTACG CTACTTTCAT CGCTTTCGCT |
| | GTGTTTGGCG GCGGTACCAA GCTGACCGTG CTGGGCCAGC CCAAAGCCGC CCCTAGCGTG |
| | ACCCTGTTCC CCCCAAGCAG CGAGGAACTC CAGGCCAACA AGGCCACCCT CGTGTGCCTG |
| | ATCAGCGACT TCTACCCTGG CGCCGTGACC GTGGCCTGGA AGGCCGATAG CAGCCCTGTG |
| | AAGGCCGGCG TGGAAACCAC CACCCCCAGC AAGCAGAGCA ACAACAAATA CGCCGCCAGC |
| | AGCTACCTGA GCCTGACCCC CGAGCAGTGG AAGTCCCACA GATCCTACAG CTGCCAGGTC |
| | ACACACGAGG GCAGCACCGT GGAAAAGACC GTGGCCCCCA CCGAGTGCAG C |
| HC Protein (SEQ ID NO: 71) | EVQLLESGGGLVQPGGSLRLSCAASGFTFSSYAMSWVRQAPGKGLEWVSAISGSGGST |
| | YYADSVKGRFTISRDNSKNTLYLQMNSLRAEDTAVYYCARGFHYTHSGKRYYYPFDIWG |
| | QGTLVTVSSASTKGPSVFPLAPSSKSTSGGTAALGCLVKDYFPEPVTVSWNSGALTSGV |
| | HTFPAVLQSSGLYSLSSVVTVPSSSLGTQTYICNVNHKPSNTKVDKRVEPKSCDKTHTCP |
| | PCPAPEAEGAPSVFLFPPKPKDTLMISRTPEVTCVVVDVSHEDPEVKFNWYVDGVEVH |
| | NAKTKPREEQYNSTYRVVSVLTVLHQDWLNGKEYKCKVSNKALPSSIEKTISKAKGQPR |
| | EPQVYTLPPSREEMTKNQVSLTCLVKGFYPSDIAVEWESNGQPENNYKTTPPVLDSDG |
| | SFFLYSKLTVDKSRWQQGNVFSCSVMHEALHNHYTQKSLSLSPGK |
| HC DNA (SEQ ID NO: 35) | GAAGTGCAGC TGCTGGAAAG CGGTGGCGGT CTGGTGCAGC CAGGTGGTAG CCTGCGCCTG |
| | AGCTGTGCCG CAAGCGGCTT TACCTTTAGC AGCTATGCCA TGAGCTGGGT GCGCCAAGCA |
| | CCAGGCAAAG GCCTGGAATG GGTGAGCGCC ATTAGCGGCA GCGGTGGCAG CACCTATTAT |
| | GCCGATAGCG TGAAAGGTCG CTTTACCATT AGTCGCGATA ACAGCAAAAA CACCCTGTAT |
| | CTGCAAATGA ACAGCCTGCG GGCAGAAGAT ACCGCAGTTT ATTATTGCGC GCGAGGATTC |
| | CACTACACTC ACTCCGGTAA ACGTTACTAC TATCCTTTTG ACATTTGGGG CCAGGGCACC |
| | CTGGTTACTG TCTCGAGCGC GTCGACCAAA GGCCCCAGCG TGTTCCCTCT GGCCCCCAGC |
| | AGCAAGAGCA CCTCTGGCGG AACAGCCGCC CTGGGCTGCC TGGTCAAGGA CTACTTCCCC |
| | GAGCCCGTGA CCGTGTCCTG GAACTCTGGC GCCCTGACCA GCGGCGTGCA CACCTTTCCA |
| | GCCGTGCTCC AGAGCAGCGG CCTGTACAGC CTGAGCAGCG TCGTGACCGT GCCCAGCAGC |
| | AGCCTGGGCA CCCAGACCTA CATCTGCAAC GTGAACCACA AGCCCAGCAA CACAAAGGTG |
| | GACAAGCGGG TGGAACCCAA GAGCTGCGAC AAGACCCACA CCTGTCCCCC CTGCCCTGCC |
| | CCTGAAGCGG AGGGAGCCCC CTCCGTGTTC CTGTTCCCCC CAAAGCCTAA GGACACCCTG |
| | ATGATCAGCC GGACCCCCGA AGTGACCTGC GTGGTGGTGG ACGTGTCCCA CGAGGACCCT |
| | GAAGTGAAGT TTAATTGGTA CGTGGACGGC GTGGAAGTGC ACAACGCCAA GACCAAGCCC |
| | AGAGAGGAAC AGTACAACAG CACCTACCGG GTGGTGTCCG TGCTGACCGT GCTGCACCAG |
| | GACTGGCTGA ACGGCAAAGA GTACAAGTGC AAGGTGTCCA ACAAGGCCCT GCCTTCCTCC |
| | ATCGAGAAAA CCATCAGCAA GGCCAAAGGC CAGCCCCGCG AGCCCCAGGT GTACACACTG |
| | CCCCCTAGCC GGGAAGAGAT GACCAAGAAC CAGGTGTCCC TGACCTGCCT CGTGAAGGGC |
| | TTCTACCCCA GCGACATTGC CGTGGAATGG GAGAGCAACG GCCAGCCCGA GAACAACTAC |
| | AAGACCACCC CCCCTGTGCT GGACAGCGAC GGCTCATTCT TCCTGTACAG CAAGCTGACC |
| | GTGGACAAGA GCCGGTGGCA GCAGGGCAAC GTGTTCAGCT GCTCCGTGAT GCACGAGGCC |
| | CTGCACAACC ACTACACCCA GAAGTCCCTG AGCCTGAGCC CCGGCAAG |

Variable Region Pair Name
Y022870

| | |
|---|---|
| Framework Vh | YLAN_VH3-23 |
| Framework Vl | YLAN_VL2-23 |
| HCDR1 (SEQ ID NO: 11) | RYAIT |
| HCDR2 (SEQ ID NO: 12) | TISALGLETYYAESVRG |
| HCDR3 (SEQ ID NO: 5) | GFHYTHSGKRYYYPFDI |
| LCDR1 (SEQ ID NO: 7) | TGTSSDVGSYNLVS |
| LCDR2 (SEQ ID NO: 8) | EGSKRPS |
| LCDR3 (SEQ ID NO: 9) | SQYATFIAFAV |
| Protein Sequence (VH) | EVQLLESGGGLVQPGGSLRLSCAASGFSFSRYAITWVRQAPGKGLEWVSTISALGLETY |
| (SEQ ID NO: 10) | YAESVRGRFTISRDNSKNTLYLQMNSLRAEDTAVYYCARGFHYTHSGKRYYYPFDIWG |
| | QGTLVTVSS |
| Protein Sequence (VL) | QSALTQPASVSGSPGQSITISCTGTSSDVGSYNLVSWYQQHPGKAPKLMIYEGSKRPSG |
| (SEQ ID NO: 6) | VSNRFSGSKSGNTASLTISGLQAEDEADYYCSQYATFIAFAVFGGGTKLTVLGQ |
| Ppt Format | h_IgG1f_AEASS |
| LC Protein | QSALTQPASVSGSPGQSITISCTGTSSDVGSYNLVSWYQQHPGKAPKLMIYEGSKRPSG |
| (SEQ ID NO: 70) | VSNRFSGSKSGNTASLTISGLQAEDEADYYCSQYATFIAFAVFGGGTKLTVLGQPKAAPS |
| | VTLFPPSSEELQANKATLVCLISDFYPGAVTVAWKADSSPVKAGVETTTPSKQSNNKYA |
| | ASSYLSLTPEQWKSHRSYSCQVTHEGSTVEKTVAPTECS |
| LC DNA (SEQ ID NO: 34) | CAGAGCGCCC TGACCCAGCC AGCCAGCGTT AGCGGTAGCC CAGGCCAGAG CATTACCATT |
| | AGCTGCACCG GCACCAGCAG CGACGTGGGC AGCTATAACC TGGTTAGCTG GTATCAGCAG |
| | CATCCGGGCA AAGCCCCGAA ACTGATGATC TATGAAGGCA GCAAACGCCC GAGCGGCGTT |
| | AGCAACCGCT TTAGTGGCAG CAAAAGCGGC AACACCGCCA GCCTGACCAT TAGCGGCCTG |
| | CAAGCCGAAG ACGAAGCCGA TTATTACTGC TCCCAGTACG CTACTTTCAT CGCTTTCGCT |
| | GTGTTTGGCG GCGGTACCAA GCTGACCGTG CTGGGCCAGC CCAAAGCCGC CCCTAGCGTG |
| | ACCCTGTTCC CCCCAAGCAG CGAGGAACTC CAGGCCAACA AGGCCACCCT CGTGTGCCTG |
| | ATCAGCGACT TCTACCCTGG CGCCGTGACC GTGGCCTGGA AGGCCGATAG CAGCCCTGTG |
| | AAGGCCGGCG TGGAAACCAC CACCCCCAGC AAGCAGAGCA ACAACAAATA CGCCGCCAGC |
| | AGCTACCTGA GCCTGACCCC CGAGCAGTGG AAGTCCCACA GATCCTACAG CTGCCAGGTC |
| | ACACACGAGG GCAGCACCGT GGAAAAGACC GTGGCCCCCA CCGAGTGCAG C |
| HC Protein (SEQ ID NO: 72) | EVQLLESGGGLVQPGGSLRLSCAASGFSFSRYAITWVRQAPGKGLEWVSTISALGLETY |
| | YAESVRGRFTISRDNSKNTLYLQMNSLRAEDTAVYYCARGFHYTHSGKRYYYPFDIWG |
| | QGTLVTVSSASTKGPSVFPLAPSSKSTSGGTAALGCLVKDYFPEPVTVSWNSGALTSGV |
| | HTFPAVLQSSGLYSLSSVVTVPSSSLGTQTYICNVNHKPSNTKVDKRVEPKSCDKTHTCP |
| | PCPAPEAEGAPSVFLFPPKPKDTLMISRTPEVTCVVVDVSHEDPEVKFNWYVDGVEVH |
| | NAKTKPREEQYNSTYRVVSVLTVLHQDWLNGKEYKCKVSNKALPSSIEKTISKAKGQPR |
| | EPQVYTLPPSREEMTKNQVSLTCLVKGFYPSDIAVEWESNGQPENNYKTTPPVLDSDG |
| | SFFLYSKLTVDKSRWQQGNVFSCSVMHEALHNHYTQKSLSLSPGK |
| HC DNA (SEQ ID NO: 36) | GAAGTGCAGC TGCTGGAAAG CGGTGGCGGT CTGGTGCAGC CAGGTGGTAG CCTGCGCCTG |
| | AGCTGTGCCG CAAGCGGGTT TTCTTTCTCC AGATACGCAA TCACCTGGGT GCGCCAAGCA |
| | CCAGGCAAAG GCCTGGAATG GGTGAGCACC ATTTCCGCAC TGGGTCTGGA GACATATTAC |
| | GCGGAGTCCG TCCGCGGCCG CTTTACCATT AGTCGCGATA ACAGCAAAAA CACCCTGTAT |
| | CTGCAAATGA ACAGCCTGCG GGCAGAAGAT ACCGCAGTTT ATTATTGCGC GCGAGGATTC |
| | CACTACACTC ACTCCGGTAA ACGTTACTAC TATCCTTTTG ACATTTGGGG CCAGGGCACC |
| | CTGGTTACTG TCTCGAGCGC GTCGACCAAA GGCCCCAGCG TGTTCCCTCT GGCCCCCAGC |
| | AGCAAGAGCA CCTCTGGCGG AACAGCCGCC CTGGGCTGCC TGGTCAAGGA CTACTTCCCC |
| | GAGCCCGTGA CCGTGTCCTG GAACTCTGGC GCCCTGACCA GCGGCGTGCA CACCTTTCCA |
| | GCCGTGCTCC AGAGCAGCGG CCTGTACAGC CTGAGCAGCG TCGTGACCGT GCCCAGCAGC |
| | AGCCTGGGCA CCCAGACCTA CATCTGCAAC GTGAACCACA AGCCCAGCAA CACAAAGGTG |
| | GACAAGCGGG TGGAACCCAA GAGCTGCGAC AAGACCCACA CCTGTCCCCC CTGCCCTGCC |
| | CCTGAAGCGG AGGGAGCCCC CTCCGTGTTC CTGTTCCCCC CAAAGCCTAA GGACACCCTG |
| | ATGATCAGCC GGACCCCCGA AGTGACCTGC GTGGTGGTGG ACGTGTCCCA CGAGGACCCT |
| | GAAGTGAAGT TTAATTGGTA CGTGGACGGC GTGGAAGTGC ACAACGCCAA GACCAAGCCC |
| | AGAGAGGAAC AGTACAACAG CACCTACCGG GTGGTGTCCG TGCTGACCGT GCTGCACCAG |
| | GACTGGCTGA ACGGCAAAGA GTACAAGTGC AAGGTGTCCA ACAAGGCCCT GCCTTCCTCC |
| | ATCGAGAAAA CCATCAGCAA GGCCAAAGGC CAGCCCCGCG AGCCCCAGGT GTACACACTG |
| | CCCCCTAGCC GGGAAGAGAT GACCAAGAAC CAGGTGTCCC TGACCTGCCT CGTGAAGGGC |
| | TTCTACCCCA GCGACATTGC CGTGGAATGG GAGAGCAACG GCCAGCCCGA GAACAACTAC |
| | AAGACCACCC CCCCTGTGCT GGACAGCGAC GGCTCATTCT TCCTGTACAG CAAGCTGACC |
| | GTGGACAAGA GCCGGTGGCA GCAGGGCAAC GTGTTCAGCT GCTCCGTGAT GCACGAGGCC |
| | CTGCACAACC ACTACACCCA GAAGTCCCTG AGCCTGAGCC CCGGCAAG |

Variable Region Pair Name
Y022877

| | |
|---|---|
| Framework Vh | YLAN_VH3-23 |
| Framework Vl | YLAN_VL2-23 |
| HCDR1 (SEQ ID NO: 14) | RYAMT |
| HCDR2 (SEQ ID NO: 15) | TISGLGQEAYYAGSVKG |
| HCDR3 (SEQ ID NO: 5) | GFHYTHSGKRYYYPFDI |
| LCDR1 (SEQ ID NO: 7) | TGTSSDVGSYNLVS |
| LCDR2 (SEQ ID NO: 8) | EGSKRPS |
| LCDR3 (SEQ ID NO: 9) | SQYATFIAFAV |
| Protein Sequence (VH) | EVQLLESGGGLVQPGGSLRLSCAASGFTFSRYAMTWVRQAPGKGLEWVSTISGLGQE |
| (SEQ ID NO: 13) | AYYAGSVKGRFTISRDNSKNTLYLQMNSLRAEDTAVYYCARGFHYTHSGKRYYYPFDIW |
| | GQGTLVTVSS |
| Protein Sequence (VL) | QSALTQPASVSGSPGQSITISCTGTSSDVGSYNLVSWYQQHPGKAPKLMIYEGSKRPSG |
| (SEQ ID NO: 6) | VSNRFSGSKSGNTASLTISGLQAEDEADYYCSQYATFIAFAVFGGGTKLTVLGQ |
| Ppt Format | h_IgG1f_AEASS |
| LC Protein | QSALTQPASVSGSPGQSITISCTGTSSDVGSYNLVSWYQQHPGKAPKLMIYEGSKRPSG |
| (SEQ ID NO: 70) | VSNRFSGSKSGNTASLTISGLQAEDEADYYCSQYATFIAFAVFGGGTKLTVLGQPKAAPS |
| | VTLFPPSSEELQANKATLVCLISDFYPGAVTVAWKADSSPVKAGVETTTPSKQSNNKYA |
| | ASSYLSLTPEQWKSHRSYSCQVTHEGSTVEKTVAPTECS |
| LC DNA | CAGAGCGCCC TGACCCAGCC AGCCAGCGTT AGCGGTAGCC CAGGCCAGAG CATTACCATT |
| (SEQ ID NO: 34) | AGCTGCACCG GCACCAGCAG CGACGTGGGC AGCTATAACC TGGTTAGCTG GTATCAGCAG |
| | CATCCGGGCA AAGCCCCGAA ACTGATGATC TATGAAGGCA GCAAACGCCC GAGCGGCGTT |
| | AGCAACCGCT TTAGTGGCAG CAAAAGCGGC AACACCGCCA GCCTGACCAT TAGCGGCCTG |
| | CAAGCCGAAG ACGAAGCCGA TTATTACTGC TCCCAGTACG CTACTTTCAT CGCTTTCGCT |
| | GTGTTTGGCG GCGGTACCAA GCTGACCGTG CTGGGCCAGC CCAAAGCCGC CCCTAGCGTG |
| | ACCCTGTTCC CCCCAAGCAG CGAGGAACTC CAGGCCAACA AGGCCACCCT CGTGTGCCTG |
| | ATCAGCGACT TCTACCCTGG CGCCGTGACC GTGGCCTGGA AGGCCGATAG CAGCCCTGTG |
| | AAGGCCGGCG TGGAAACCAC CACCCCCAGC AAGCAGAGCA ACAACAAATA CGCCGCCAGC |
| | AGCTACCTGA GCCTGACCCC CGAGCAGTGG AAGTCCCACA GATCCTACAG CTGCCAGGTC |
| | ACACACGAGG GCAGCACCGT GGAAAAGACC GTGGCCCCCA CCGAGTGCAG C |
| HC Protein | EVQLLESGGGLVQPGGSLRLSCAASGFTFSRYAMTWVRQAPGKGLEWVSTISGLGQE |
| (SEQ ID NO: 73) | AYYAGSVKGRFTISRDNSKNTLYLQMNSLRAEDTAVYYCARGFHYTHSGKRYYYPFDIW |
| | GQGTLVTVSSASTKGPSVFPLAPSSKSTSGGTAALGCLVKDYFPEPVTVSWNSGALTSG |
| | VHTFPAVLQSSGLYSLSSVVTVPSSSLGTQTYICNVNHKPSNTKVDKRVEPKSCDKTHTC |
| | PPCPAPEAEGAPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPSSIEKTISKAKGQP |
| | REPQVYTLPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |
| HC DNA | GAAGTGCAGC TGCTGGAAAG CGGTGGCGGT CTGGTGCAGC CAGGTGGTAG CCTGCGCCTG |
| (SEQ ID NO: 37) | AGCTGTGCCG CAAGCGGCTT TACCTTCAGC AGATATGCAA TGACTTGGGT GCGCCAAGCA |
| | CCAGGCAAAG GCCTGGAATG GGTGAGTACC ATAAGCGGGC TGGGACAAGA GGCTTACTAC |
| | GCAGGCTCCG TCAAAGGCCG CTTTACCATT AGTCGCGATA ACAGCAAAAA CACCCTGTAT |
| | CTGCAAATGA ACAGCCTGCG GGCAGAAGAT ACCGCAGTTT ATTATTGCGC GCGAGGATTC |
| | CACTACACTC ACTCCGGTAA ACGTTACTAC TATCCTTTTG ACATTTGGGG CCAGGGCACC |
| | CTGGTTACTG TCTCGAGCGC GTCGACCAAA GGCCCCAGCG TGTTCCCTCT GGCCCCCAGC |
| | AGCAAGAGCA CCTCTGGCGG AACAGCCGCC CTGGGCTGCC TGGTCAAGGA CTACTTCCCC |
| | GAGCCCGTGA CCGTGTCCTG GAACTCTGGC GCCCTGACCA GCGGCGTGCA CACCTTTCCA |
| | GCCGTGCTCC AGAGCAGCGG CCTGTACAGC CTGAGCAGCG TCGTGACCGT GCCCAGCAGC |
| | AGCCTGGGCA CCCAGACCTA CATCTGCAAC GTGAACCACA AGCCCAGCAA CACAAAGGTG |
| | GACAAGCGGG TGGAACCCAA GAGCTGCGAC AAGACCCACA CCTGTCCCCC CTGCCCTGCC |
| | CCTGAAGCGG AGGGAGCCCC CTCCGTGTTC CTGTTCCCCC CAAAGCCTAA GGACACCCTG |
| | ATGATCAGCC GGACCCCCGA AGTGACCTGC GTGGTGGTGG ACGTGTCCCA CGAGGACCCT |
| | GAAGTGAAGT TTAATTGGTA CGTGGACGGC GTGGAAGTGC ACAACGCCAA GACCAAGCCC |
| | AGAGAGGAAC AGTACAACAG CACCTACCGG GTGGTGTCCG TGCTGACCGT GCTGCACCAG |
| | GACTGGCTGA ACGGCAAAGA GTACAAGTGC AAGGTGTCCA ACAAGGCCCT GCCTTCCTCC |
| | ATCGAGAAAA CCATCAGCAA GGCCAAAGGC CAGCCCCGCG AGCCCCAGGT GTACACACTG |
| | CCCCCTAGCC GGGAAGAGAT GACCAAGAAC CAGGTGTCCC TGACCTGCCT CGTGAAGGGC |
| | TTCTACCCCA GCGACATTGC CGTGGAATGG GAGAGCAACG GCCAGCCCGA GAACAACTAC |
| | AAGACCACCC CCCCTGTGCT GGACAGCGAC GGCTCATTCT TCCTGTACAG CAAGCTGACC |
| | GTGGACAAGA GCCGGTGGCA GCAGGGCAAC GTGTTCAGCT GCTCCGTGAT GCACGAGGCC |
| | CTGCACAACC ACTACACCCA GAAGTCCCTG AGCCTGAGCC CCGGCAAG |

Variable Region Pair Name
Y022883

| | |
|---|---|
| Framework Vh | YLAN_VH3-23 |
| Framework Vl | YLAN_VL2-23 |
| HCDR1 (SEQ ID NO: 17) | RYAIS |
| HCDR2 (SEQ ID NO: 18) | VISGHGSEANYAESVKG |
| HCDR3 (SEQ ID NO: 5) | GFHYTHSGKRYYYPFDI |
| LCDR1 (SEQ ID NO: 7) | TGTSSDVGSYNLVS |
| LCDR2 (SEQ ID NO: 8) | EGSKRPS |
| LCDR3 (SEQ ID NO: 9) | SQYATFIAFAV |
| Protein Sequence (VH) | EVQLLESGGGLVQPGGSLRLSCAASGFSFTRYAISWVRQAPGKGLEWVSVISGHGSEAN |
| (SEQ ID NO: 16) | YAESVKGRFTISRDNSKNTLYLQMNSLRAEDTAVYYCARGFHYTHSGKRYYYPFDIWGQ |
| | GTLVTVSS |
| Protein Sequence (VL) | QSALTQPASVSGSPGQSITISCTGTSSDVGSYNLVSWYQQHPGKAPKLMIYEGSKRPSG |
| (SEQ ID NO: 6) | VSNRFSGSKSGNTASLTISGLQAEDEADYYCSQYATFIAFAVFGGGTKLTVLGQ |
| Ppt Format | h_IgG1f_AEASS |
| LC Protein | QSALTQPASVSGSPGQSITISCTGTSSDVGSYNLVSWYQQHPGKAPKLMIYEGSKRPSG |
| (SEQ ID NO: 70) | VSNRFSGSKSGNTASLTISGLQAEDEADYYCSQYATFIAFAVFGGGTKLTVLGQPKAAPS |
| | VTLFPPSSEELQANKATLVCLISDFYPGAVTVAWKADSSPVKAGVETTTPSKQSNNKYAA |
| | SSYLSLTPEQWKSHRSYSCQVTHEGSTVEKTVAPTECS |
| LC DNA (SEQ ID NO: 34) | CAGAGCGCCC TGACCCAGCC AGCCAGCGTT AGCGGTAGCC CAGGCCAGAG CATTACCATT |
| | AGCTGCACCG GCACCAGCAG CGACGTGGGC AGCTATAACC TGGTTAGCTG GTATCAGCAG |
| | CATCCGGGCA AAGCCCCGAA ACTGATGATC TATGAAGGCA GCAAACGCCC GAGCGGCGTT |
| | AGCAACCGCT TTAGTGGCAG CAAAAGCGGC AACACCGCCA GCCTGACCAT TAGCGGCCTG |
| | CAAGCCGAAG ACGAAGCCGA TTATTACTGC TCCCAGTACG CTACTTTCAT CGCTTTCGCT |
| | GTGTTTGGCG GCGGTACCAA GCTGACCGTG CTGGGCCAGC CCAAAGCCGC CCCTAGCGTG |
| | ACCCTGTTCC CCCCAAGCAG CGAGGAACTC CAGGCCAACA AGGCCACCCT CGTGTGCCTG |
| | ATCAGCGACT TCTACCCTGG CGCCGTGACC GTGGCCTGGA AGGCCGATAG CAGCCCTGTG |
| | AAGGCCGGCG TGGAAACCAC CACCCCCAGC AAGCAGAGCA ACAACAAATA CGCCGCCAGC |
| | AGCTACCTGA GCCTGACCCC CGAGCAGTGG AAGTCCCACA GATCCTACAG CTGCCAGGTC |
| | ACACACGAGG GCAGCACCGT GGAAAAGACC GTGGCCCCCA CCGAGTGCAG C |
| HC Protein | EVQLLESGGGLVQPGGSLRLSCAASGFSFTRYAISWVRQAPGKGLEWVSVISGHGSEAN |
| (SEQ ID NO: 74) | YAESVKGRFTISRDNSKNTLYLQMNSLRAEDTAVYYCARGFHYTHSGKRYYYPFDIWGQ |
| | GTLVTVSSASTKGPSVFPLAPSSKSTSGGTAALGCLVKDYFPEPVTVSWNSGALTSGVHT |
| | FPAVLQSSGLYSLSSVVTVPSSSLGTQTYICNVNHKPSNTKVDKRVEPKSCDKTHTCPPCP |
| | APEAEGAPSVFLFPPKPKDTLMISRTPEVTCVVVDVSHEDPEVKFNWYVDGVEVHNAK |
| | TKPREEQYNSTYRVVSVLTVLHQDWLNGKEYKCKVSNKALPSSIEKTISKAKGQPREPQV |
| | YTLPPSREEMTKNQVSLTCLVKGFYPSDIAVEWESNGQPENNYKTTPPVLDSDGSFFLYS |
| | KLTVDKSRWQQGNVFSCSVMHEALHNHYTQKSLSLSPGK |
| HC DNA | GAAGTGCAGC TGCTGGAAAG CGGTGGCGGT CTGGTGCAGC CAGGTGGTAG CCTGCGCCTG |
| (SEQ ID NO: 38) | AGCTGTGCCG CAAGCGGGTT CTCCTTCACC AGATATGCAA TTTCCTGGGT GCGCCAAGCA |
| | CCAGGCAAAG GCCTGGAATG GGTGAGTGTG ATAAGCGGGC ATGGTTCCGA GGCTAACTAC |
| | GCCGAGAGCG TCAAGGGTCG CTTTACCATT AGTCGCGATA ACAGCAAAAA CACCCTGTAT |
| | CTGCAAATGA ACAGCCTGCG GGCAGAAGAT ACCGCAGTTT ATTATTGCGC GCGAGGATTC |
| | CACTACACTC ACTCCGGTAA ACGTTACTAC TATCCTTTTG ACATTTGGGG CCAGGGCACC |
| | CTGGTTACTG TCTCGAGCGC GTCGACCAAA GGCCCCAGCG TGTTCCCTCT GGCCCCCAGC |
| | AGCAAGAGCA CCTCTGGCGG AACAGCCGCC CTGGGCTGCC TGGTCAAGGA CTACTTCCCC |
| | GAGCCCGTGA CCGTGTCCTG GAACTCTGGC GCCCTGACCA GCGGCGTGCA CACCTTTCCA |
| | GCCGTGCTCC AGAGCAGCGG CCTGTACAGC CTGAGCAGCG TCGTGACCGT GCCCAGCAGC |
| | AGCCTGGGCA CCCAGACCTA CATCTGCAAC GTGAACCACA AGCCCAGCAA CACAAAGGTG |
| | GACAAGCGGG TGGAACCCAA GAGCTGCGAC AAGACCCACA CCTGTCCCCC CTGCCCTGCC |
| | CCTGAAGCGG AGGGAGCCCC CTCCGTGTTC CTGTTCCCCC CAAAGCCTAA GGACACCCTG |
| | ATGATCAGCC GGACCCCCGA AGTGACCTGC GTGGTGGTGG ACGTGTCCCA CGAGGACCCT |
| | GAAGTGAAGT TTAATTGGTA CGTGGACGGC GTGGAAGTGC ACAACGCCAA GACCAAGCCC |
| | AGAGAGGAAC AGTACAACAG CACCTACCGG GTGGTGTCCG TGCTGACCGT GCTGCACCAG |
| | GACTGGCTGA ACGGCAAAGA GTACAAGTGC AAGGTGTCCA ACAAGGCCCT GCCTTCCTCC |
| | ATCGAGAAAA CCATCAGCAA GGCCAAAGGC CAGCCCCGCG AGCCCCAGGT GTACACACTG |
| | CCCCCTAGCC GGGAAGAGAT GACCAAGAAC CAGGTGTCCC TGACCTGCCT CGTGAAGGGC |
| | TTCTACCCCA GCGACATTGC CGTGGAATGG GAGAGCAACG GCCAGCCCGA GAACAACTAC |
| | AAGACCACCC CCCCTGTGCT GGACAGCGAC GGCTCATTCT TCCTGTACAG CAAGCTGACC |
| | GTGGACAAGA GCCGGTGGCA GCAGGGCAAC GTGTTCAGCT GCTCCGTGAT GCACGAGGCC |
| | CTGCACAACC ACTACACCCA GAAGTCCCTG AGCCTGAGCC CCGGCAAG |

### Table 24: Sequence Listing Key

| SEQ ID | | |
|---|---|---|
| NO: | Descriptor | Sequence |
| 1 | Wildtype human PAR2 | MRSPSAAWLLGAAILLAASLSCSGTIQGTNRSSKGRSLIGKVDGTSHVTGKGVTVETVFSVDEFSASVLTGKLTTVFLPIVYTIVFVVGLPSNGMALW |
| | | VFLFRTKKKHPAVIYMANLALADLLSVIWFPLKIAYHIHGNNWIYGEALCNVLIGFFYGNMYCSILFMTCLSVQRYWVIVNPMGHSRKKANIAIGISL |
| | | AIWLLILLVTIPLYVVKQTIFIPALNITTCHDVLPEQLLVGDMFNYFLSLAIGVFLFPAFLTASAYVLMIRMLRSSAMDENSEKKRKRAIKLIVTVLAMYLI |
| | | CFTPSNLLLVVHYFLIKSQGQSHVYALYIVALCLSTLNSCIDPFVYYFVSHDFRDHAKNALLCRSVRTVKQMQVSLTSKKHSRKSSSYSSSSTTVKTSY |
| 2 | VH region of clone Y022065 | See table 20 |
| 3 | HCDR1 region of clone | See table 20 |
| | Y022065 according to Kabat | |
| 4 | HCDR2 region of clone | See table 20 |
| | Y022065 according to Kabat | |
| 5 | HCDR3 region of clones | See tables 20-23 |
| | Y022065, Y022870, Y022877 | |
| | and Y022883 according to | |
| | Kabat | |
| 6 | VL region of clones Y022065, | See tables 20-23 |
| | Y022870, Y022877 and | |
| | Y022883 | |
| 7 | LCDR1 region of clones | See tables 20-23 |
| | Y022065, Y022870, Y022877 | |
| | and Y022883 according to | |
| | Kabat | |
| 8 | LCDR2 region of clones | See tables 20-23 |
| | Y022065, Y022870, Y022877 | |
| | and Y022883 according to | |
| | Kabat | |
| 9 | LCDR3 region of clones | See tables 20-23 |
| | Y022065, Y022870, Y022877 | |
| | and Y022883 according to | |
| | Kabat | |
| 10 | VH region of clone Y022870 | See table 21 |
| 11 | HCDR1 region of clone | See table 21 |
| | Y022870 according to Kabat | |
| 12 | HCDR2 region of clone | See table 21 |
| | Y022870 according to Kabat | |
| 13 | VH region of clone Y022877 | See table 22 |
| 14 | HCDR1 region of clone | See table 22 |
| | Y022877 according to Kabat | |
| 15 | HCDR2 region of clone | See table 22 |
| | Y022877 according to Kabat | |
| 16 | VH region of clone Y022883 | See table 23 |
| 17 | HCDR1 region of clone | See table 23 |
| | Y022883 according to Kabat | |
| 18 | HCDR2 region of clone | See table 23 |
| | Y022883 according to Kabat | |
| 19 | VH region of clone Y022054 | EVQLLESGGGLVQPGGSLRLSCAASGFTFSSYAISWVRQAPGKGLEWVSAISGSGGSTYYAESVKGRFTISRDNSKNTLYLQMNSLRAEDTAVYYCA |
| | | RGYAEFGVWGQGTLVTVSS |
| 20 | HCDR1 region of clone | SYAIS |
| | Y022054 according to Kabat | |
| 21 | HCDR2 region of clone | AISGSGGSTYYAESVKG |
| | Y022054 according to Kabat | |
| 22 | HCDR3 region of clone | GYAEFGV |
| | Y022054 according to Kabat | |
| 23 | VL region of clone Y022054 | DIQMTQSPSSLSASVGDRVTITCRASQSISSYLNWYQQKPGKAPKLLIYAASSLQSGVPSRFSGSGSGTDFTLTISSLQPEDFATYYCQQTWGGPQTF |
| | | GQGTKVEIKRT |
| 24 | LCDR1 region of clone Y022054 | RASQSISSYLN |
| | according to Kabat | |
| 25 | LCDR2 region of clones | AASSLQS |
| | Y022054 and Y021171 | |
| | according to Kabat | |
| 26 | LCDR3 region of clone Y022054 | QQTWGGPQT |
| | according to Kabat | |
| 27 | VH region of clone Y021171 | EVQLVESGGGLVKPGGSLRLSCAASGFTFSSYSINWVRQAPGKGLEWVSSISSSSSYTYYAESVKGRFTISRDNAKNSLYLQMNSLRAEDTAVYYCA |
| | | RAASGFAIFDYWGQGTLVTVSS |
| 28 | HCDR1 region of clone | SYSIN |
| | Y021171 according to Kabat | |
| 29 | HCDR2 region of clone | SISSSSSYTYYAESVKG |
| | Y021171 according to Kabat | |
| 30 | HCDR3 region of clone | AASGFAIFDY |
| | Y021171 according to Kabat | |
| 31 | VL region of clone Y021171 | DIQMTQSPSSVSASVGDRVTITCRASQGISSWLAWYQQKPGKAPKLLIYAASSLQSGVPSRFSGSGSGTDFTLTISSLQPEDFATYYCQQTHYVPLTF |
| | | GQGTKVEIKRT |
| 32 | LCDR1 region of clone Y021171 | RASQGISSWLA |
| | according to Kabat | |
| 33 | LCDR3 region of clone Y021171 | QQTHYVPLT |
| | according to Kabat | |
| 34 | LC nucleotide sequence of | See tables 20-23 |
| | Y022065, Y022870, Y022877 | |
| | and Y022883 | |
| 35 | HC nucleotide sequence of | See table 20 |
| | Y022065 | |
| 36 | HC nucleotide sequence of | See table 21 |
| | Y022870 | |
| 37 | HC nucleotide sequence of | See table 22 |
| | Y022877 | |
| 38 | HC nucleotide sequence of | See table 23 |
| | Y022883 | |
| 39 | Human PAR2 activated peptide | SLIGKV |
| 40 | Murine PAR2 activated peptide | SLIGRL |
| 41 | PAR2 epitope sequence 1 | VETVE |
| | encompassing N-terminus | |
| 42 | PAR2 epitope sequence 2 | SVDEFSASVLTGKLTTVF |
| | encompassing Helix0/1 | |
| 43 | PAR2 epitope sequence 3 | LLVVHY |
| | encompassing ECL3 | |
| 44 | PAR2 epitope sequence 4 | FLIKSQGQSHVYALY |
| | encompassing ECL3 | |
| 45 | PAR2 N-terminal region | VTVETVFSVDEFSASVLTGKLTTV |
| 46 | HCDR1 region of clone | GFTFSSY |
| | Y022065 according to Chothia | |
| 47 | HCDR2 region of clone | SGSGGS |
| | Y022065 according to Chothia | |
| 48 | HCDR3 region of clones | GFHYTHSGKRYYYPFDI |
| | Y022065, Y022870, Y022877 | |
| | and Y022883 according to | |
| | Chothia | |
| 49 | LCDR1 region of clones | TSSDVGSYNL |
| | Y022065, Y022870, Y022877 | |
| | and Y022883 according to | |
| | Chothia | |
| 50 | LCDR3 region of clones | YATFIAFA |
| | Y022065, Y022870, Y022877 | |
| | and Y022883 according to | |
| | Chothia | |
| 51 | HCDR1 region of clone | GFSFSRY |
| | Y022870 according to Chothia | |
| 52 | HCDR2 region of clone | SALGLE |
| | Y022870 according to Chothia | |
| 53 | HCDR1 region of clone | GFTFSRY |
| | Y022877 according to Chothia | |
| 54 | HCDR2 region of clone | SGLGQE |
| | Y022877 according to Chothia | |
| 55 | HCDR1 region of clone | GFSFTRY |
| | Y022883 according to Chothia | |
| 56 | HCDR2 region of clone | SGHGSE |
| | Y022883 according to Chothia | |
| 57 | HCDR1 region of clone | GFTFSSY |
| | Y022054 according to Chothia | |
| 58 | HCDR2 region of clone | SGSGGS |
| | Y022054 according to Chothia | |
| 59 | HCDR3 region of clone | GYAEFGV |
| | Y022054 according to Chothia | |
| 60 | LCDR1 region of clone Y022054 | SQSISSY |
| | according to Chothia | |
| 61 | LCDR3 region of clone Y022054 | WGGPQ |
| | according to Chothia | |
| 62 | HCDR1 region of clone | GFTFSSY |
| | Y021171 according to Chothia | |
| 63 | HCDR2 region of clone | SSSSSY |
| | Y021171 according to Chothia | |
| 64 | HCDR3 region of clone | AASGFAIFDY |
| | Y021171 according to Chothia | |
| 65 | LCDR1 region of clone Y021171 | SQGISSW |
| | according to Chothia | |
| 66 | LCDR3 region of clone Y021171 | HYVPL |
| | according to Chothia | |
| 67 | Wildtype human PAR 1 | MGPRRLLLVAACFSLCGPLLSARTRARRPESKATNATLDPRSFLLRNPNDKYEPFWEDEEKNESGLTEYRLVSINKSSPLQKQLPAFISEDASGYLTSS |
| | | WLTLFVPSVYTGVFVVSLPLNIMAIVVFILKMKVKKPAVVYMLHLATADVLFVSVLPFKISYYFSGSDWQFGSELCRFVTAAFYCNMYASILLMTVISI |
| | | DRFLAVVYPMQSLSWRTLGRASFTCLAIWALAIAGVVPLLLKEQTIQVPGLNITTCHDVLNETLLEGYYAYYFSAFSAVFFFVPLIISTVCYVSIIRCLSSS |
| | | AVANRSKKSRALFLSAAVFCIFIICFGPTNVLLIAHYSFLSHTSTTEAAYFAYLLCVCVSSISCCIDPLIYYYASSECQRYVYSILCCKESSDPSSYNSSGQLM |
| | | ASKMDTCSSNLNNSIYKKLLT |
| 68 | Wildtype human PAR 3 | MKALIFAAAGLLLLLPTFCQSGMENDTNNLAKPTLPIKTFRGAPPNSFEEFPFSALEGWTGATITVKIKCPEESASHLHVKNATMGYLTSSLSTKLIPAI |
| | | YLLVFVVGVPANAVTLWMLFFRTRSICTTVFYTNLAIADFLFCVTLPFKIAYHLNGNNWVFGEVLCRATTVIFYGNMYCSILLLACISINRYLAIVHPFT |
| | | YRGLPKHTYALVTCGLVWATVFLYMLPFFILKQEYYLVQPDITTCHDVHNTCESSSPFQLYYFISLAFFGFLIPFVLIIYCYAAIIRTLNAYDHRWLWYVK |
| | | ASLLILVIFTICFAPSNIILIIHHANYYYNNTDGLYFIYLIALCLGSLNSCLDPFLYFLMSKTRNHSTAYLTK |
| 69 | Wildtype human PAR 4 | MWGRLLLWPLVLGFSLSGGTQTPSVYDESGSTGGGDDSTPSILPAPRGYPGQVCANDSDTLELPDSSRALLLGWVPTRLVPALYGLVLVVGLPAN |
| | | GLALWVLATQAPRLPSTMLLMNLAAADLLLALALPPRIAYHLRGQRWPFGEAACRLATAALYGHMYGSVLLLAAVSLDRYLALVHPLRARALRGR |
| | | RLALGLCMAAWLMAAALALPLTLQRQTFRLARSDRVLCHDALPLDAQASHWQPAFTCLALLGCFLPLLAMLLCYGATLHTLAASGRRYGHALRLT |
| | | AVVLASAVAFFVPSNLLLLLHYSDPSPSAWGNLYGAYVPSLALSTLNSCVDPFIYYYVSAEFRDKVRAGLFQRSPGDTVASKASAEGGSRGMGTHSS |
| | | LLQ |
| 70 | LC amino acid sequence of | See tables 20-23 |
| | Y022065, Y022870, Y022877 | |
| | and Y022883 | |
| 71 | HC amino acid sequence of | See table 20 |
| | Y022065 | |
| 72 | HC amino acid sequence of | See table 21 |
| | Y022870 | |
| 73 | HC amino acid sequence of | See table 22 |
| | Y022877 | |
| 74 | HC amino acid sequence of | See table 23 |
| | Y022883 | |

### REFERENCES

Abbas, A., Lichtman, A. and Pillai, S., 2000. Cellular and molecular immunology. 4th ed.
Allen L. V. 2008. The Art, Science, and Technology of Pharmaceutical Compounding, Third Edition.
Amadesi, S., Cottrell, G. S., Divino, L., Chapman, K., Grady, E. F., Bautista, F., Karanjia, R., Barajas-Lopez, C., Vanner, S., Vergnolle, N. and Bunnett, N. W. 2006. Protease-activated receptor 2 sensitizes TRPV1 by protein kinase Cε- and A-dependent mechanisms in rats and mice. J. Physiol, 575(2), pp. 555-571.
Amiable, N., Tat, S., Lajeunesse, D., Duval, N., Pelletier, J., Martel-Pelletier, J. and Boileau, C., 2009. Proteinase-activated receptor (PAR)-2 activation impacts bone resorptive properties of human osteoarthritic subchondral bone osteoblasts. Bone, 44(6), pp. 1143-1150.
Ausubel, F., 1987. Current protocols in molecular biology.
Banerjee, S., Huber, T. and Sakmar, T., 2008. Rapid Incorporation of Functional Rhodopsin into Nanoscale Apolipoprotein Bound Bilayer (NABB) Particles. Journal of Molecular Biology, 377(4), pp. 1067-1081.
Bird R E, Hardman K D, Jacobson J W, Johnson S, Kaufman B M, Lee S M, Lee T, Pope S H, Riordan G S, Whitlow M. Science. 1988. Single-chain antigen-binding proteins. Science, 242(4877), pp. 423-6.
Cheng, R., Fiez-Vandal, C., Schlenker, O., Edman, K., Aggeler, B., Brown, D., Brown, G., Cooke, R., Dumelin, C., Doré, A., Geschwindner, S., Grebner, C., Hermansson, N., Jazayeri, A., Johansson, P., Leong, L., Prihandoko, R., Rappas, M., Soutter, H., Snijder, A., Sundström, L., Tehan, B., Thornton, P., Troast, D., Wiggin, G., Zhukov, A., Marshall, F. and Dekker, N., 2017. Structural insight into allosteric modulation of protease-activated receptor 2. Nature, 545(7652), pp. 112-115.
Dai Y, Wang S, Tominaga M, Yamamoto S, Fukuoka T, Higashi T, Kobayashi K, Obata K, Yamanaka H, Noguchi K. 2007. Sensitization of TRPA1 by PAR2 contributes to the sensation of inflammatory pain. J Clin Invest. 117(7), pp 1979-87.
Frese, K., Eisenmann, M.; Ostendorp, R.; Brocks, B.; Pabst, S. 2013. An automated immunoassay for early specificity profiling of antibodies. mAbs 5(2), pp. 279-287
Gawinowicz, M. A., Merlini, G., Birken, S., Osserman, E. F., Kabat, E. A. 1991. Amino acid sequence of the FV region of a human monoclonal IgM (NOV) with specificity for the capsular polysaccharide of the group B meningococcus and of Escherichia coli K1, which cross-reacts with polynucleotides and with denatured DNA. J Immunol 147(3), pp. 915-920.
Giblin P, Boxhammer R, Desai S, Kroe-Barrett R, Hansen G, Ksiazek J, Panzenbeck M, Ralph K, Schwartz R, Zimmitti C, Pracht C, Miller S, Magram J, Litzenburger T. 2011. Fully human antibodies against the Protease-Activated Receptor-2 (PAR-2) with anti-inflammatory activity. Hum Antibodies. 20(3-4):83-94.
Hamilton J R, Trejo J. 2017. Challenges and Opportunities in Protease-Activated Receptor Drug Development. Annu Rev Pharmacol Toxicol. 57, pp. 349-373.
Hawkins, R. E.; Russell, S. J.; Winter, G. 1992: Selection of phage antibodies by binding affinity. Mimicking affinity maturation. J. Mol. Biol. 226(3), pp. 889-896.
Helamo, D., Delil, R. and Dileba, T. 2017. Trends of diabetes mellitus and hypertension at Nigist Eleni Mohammed General Hospital, Hossana, Ethiopia (December 2010-January 2014): a five year longitudinal study. Safety in Health, 3(1).
Heuberger, D. and Schuepbach, R., 2019. Protease-activated receptors (PARs): mechanisms of action and potential therapeutic modulators in PAR-driven inflammatory diseases. Thrombosis Journal, 17(4).
Huston, J., Levinson, D., Mudgett-Hunter, M., Tai, M., Novotny, J., Margolies, M., Ridge, R., Bruccoleri, R., Haber, E. and Crea, R., 1988. Protein engineering of antibody binding sites: recovery of specific activity in an anti-digoxin single-chain Fv analogue produced in Escherichia coli. Proceedings of the National Academy of Sciences, 85(16), pp. 5879-5883.
Hutchison, C., Phillips, S., Edgell, M., Gillam, S., Jahnke, P. and Smith, M., 1978. Mutagenesis at a specific position in a DNA sequence. Journal of Biological Chemistry, 253(18), pp. 6551-6560.
Kagota S, Maruyama K, McGuire J J. 2016. Characterization and Functions of Protease-Activated Receptor 2 in Obesity, Diabetes, and Metabolic Syndrome: A Systematic Review. Biomed Res Int. 2016:3130496
Lindner, J., Kahn, M., Coughlin, S., Sambrano, G., Schauble, E., Bernstein, D., Foy, D., Hafezi-Moghadam, A. and Ley, K., 2000. Delayed Onset of Inflammation in Protease-Activated Receptor-2-Deficient Mice. The Journal of Immunology, 165(11), pp. 6504-6510.
Lohman R J, Cotterell A J, Barry G D, Liu L, Suen J Y, Vesey D A, Fairlie D P. 2012. An antagonist of human protease activated receptor-2 attenuates PAR2 signaling, macrophage activation, mast cell degranulation, and collagen-induced arthritis in rats. FASEB J. 26(7), pp. 2877-87.
Lohning, C. Novel methods for displaying (poly) peptides/proteins on bacteriophage particles via disulfide bonds. Applied for by MorphoSys AG. Patent no. WO 01/05950 A2
Low, N. M.; Hollinger, P.; Winter, G. (1996): Mimicking Somatic Hypermutation: Affinity maturation of Antibodies Displayed on Bacteriophage Using a Bacterial Mutator Strain. J. Mol. Biol. 260 (3), pp. 359-368.
Mrozkova P, Palecek J, Spicarova D. 2016. The role of protease-activated receptor type 2 in nociceptive signaling and pain. Physiol Res. 65(3), pp. 357-67.
Prassler, J., Steidl, S., Urlinger, S. 2009. In vitro affinity maturation of HuCAL antibodies: complementarity determining region exchange and RapMAT technology. Immunotherapy 1 (4), pp. 571-583.
Rice, A. S. C., Smith, B. H. and Blyth, F. M. 2016. Pain and the global burden of disease. PAIN, 157(4), pp. 791-796.
Saiki, R., Scharf, S., Faloona, F., Mullis, K., Horn, G., Erlich, H. and Arnheim, N., 1985. Enzymatic amplification of beta-globin genomic sequences and restriction site analysis for diagnosis of sickle cell anemia. Science, 230(4732), pp. 1350-1354.
Smith, G., Summers, M. and Fraser, M., 1983. Production of human beta interferon in insect cells infected with a baculovirus expression vector. Molecular and Cellular Biology, 3(12), pp. 2156-2165.
Taylor, L., Carmack, C., Schramm, S., Mashayekh, R., Higgins, K., Kuo, C., Woodhouse, C., Kay, R. and Lonberg, N., 1992. A transgenic mouse that expresses a diversity of human sequence heavy and light chain immunoglobulins. Nucleic Acids Research, 20(23), pp. 6287-6295.
Tiller T, Schuster I, Deppe D, Siegers K, Strohner R, Herrmann T, Berenguer M, Poujol D, Stehle J, Stark Y, Heßling M, Daubert D, Felderer K, Kaden S, Kölln J, Enzelberger M, Urlinger S. 2013. A fully synthetic human Fab antibody library based on fixed VH/VL framework pairings with favorable biophysical properties. MAbs. 5(3), pp. 445-70.
Van den Brulle J, Fischer M, Langmann T, Horn G, Waldmann T, Arnold S, Fuhrmann M, Schatz O, O'Connell T, O'Connell D, Auckenthaler A, Schwer H. 2008. A novel solid phase technology for high-throughput gene synthesis. Biotechniques. 45(3), pp. 340-3.
Wagner F, Lange C F, Nowak M, Ignatenko S. 2014. FRI0315 First Human Dose of the Anti-C5a Receptor-Targeting, Human Monoclonal Antibody NNC0215-0384 in Patients with Rheumatoid Arthritis: A Phase 1, Randomised, Double-Blind, Single-Dose, Dose-Escalation Trial. Annals of the Rheumatic Diseases, 73 (Supplement 2), abstract FRI0315. http://dx.doi.org/10.1136/annrheumdis-2014-eular.2156
Yamaguchi R, Yamamoto T, Sakamoto A, Narahara S, Sugiuchi H, Yamaguchi Y. 2016. Neutrophil elastase enhances IL-12p40 production by lipopolysaccharide-stimulated macrophages via transactivation of the PAR-2/EGFR/TLR4 signaling pathway. Blood Cells Mol Dis. 59, pp. 1-7.
Yau M K, Liu L, Fairlie D P. 2013. Toward drugs for protease-activated receptor 2 (PAR2). J Med Chem. 56 (19), pp. 7477-97.
Yau M K, Lim J, Liu L, Fairlie D P. 2016. Protease activated receptor 2 (PAR2) modulators: a patent review (2010-2015). Expert Opin Ther. Pat. 26(4), pp. 471-83.

The invention is not to be limited in scope by the specific aspects described herein. Indeed, various modifications of the invention in addition to those described will become apparent to those skilled in the art from the foregoing description and accompanying figures. Such modifications are intended to fall within the scope of the appended claims. All references (e.g., publications or patents or patent applications) cited herein are incorporated herein by reference in their entirety.

1. (canceled)

2. (canceled)

3. An antibody or antigen-binding fragment thereof comprising a V_{H} domain, the V_{H} domain comprising a HCDR3 selected from:
(a) a HCDR3 comprising or consisting of the amino acid sequence of SEQ ID NOs: 5, 22 or 30; or SEQ ID NO: 5, 22 or 30 with 3, 2 or 1 amino acid substitutions thereto;
(b) a HCDR3 comprising an amino acid sequence that is at least 80%, 85%, 90%, 92%, 93%, 95%, 97%, 98%, 99% or 100% identical to SEQ ID NOs: 5, 22 or 30; or
(c) a HCDR3 amino acid sequence is as defined by Kabat or Chothia and is from a V_{H} domain selected from SEQ ID NOs: 2, 10, 13, 16, 19 or 27.

4. The antibody or antigen binding fragment thereof according to claim 3, wherein the V_{H} domain comprises:
i. a HCDR1 amino acid sequence selected from SEQ ID NOs: 3, 11, 14, 17, 20 or 28, or SEQ ID NO: 3, 11, 14, 17, 20 or 28 with 3, 2 or 1 amino acid substitution(s) thereto; or
ii. a HCDR2 amino acid sequence selected from SEQ ID NOs: 4, 12, 15, 18, 21 or 29, or SEQ ID NOs: 4, 12, 15, 18, 21 or 29 with 3, 2 or 1 amino acid substitution(s) thereto.

5. The antibody or antigen binding fragment thereof according to claim 3, which further comprises a V_{L} domain comprising an amino acid sequence that is at least 80%, 85%, 90%, 92%, 93%, 95%, 97%, 98%, 99% or 100% identical to SEQ ID NOs: 6, 23 or 31.

6. The antibody or fragment thereof according to claim 3, comprising a LCDR3, wherein
a. the LCDR3 amino acid sequence is selected from SEQ ID NO: 9, 26 or 33, or SEQ ID NO: 9, 26 or 33 with 3, 2 or 1 amino acid substitution(s) thereto; or
b. the LCDR3 amino acid sequence as defined by Chothia or Kabat and from a V_{L} domain according to SEQ ID NO: 6, 23 or 31, or SEQ ID NO: 6, 23 or 31 with 3, 2 or 1 amino acid substitution(s) thereto.

7. The antibody or antigen binding fragment thereof according to claim 5, wherein the V_{L} domain comprises:
a) i) a LCDR1 amino acid sequence of SEQ ID NOs: 7, 24 or 32, or SEQ ID NOs: 7, 24 or 32 with 3, 2 or 1 amino acid substitution(s) thereto; or
ii) a LCDR1 amino acid sequence, as defined by Chothia or Kabat, from a V_{L} domain according to SEQ ID Nos: 6, 23 or 31, or SEQ ID Nos: 6, 23 or 31 with 3, 2 or 1 amino acid substitution(s) thereto; or
b) i) a LCDR2 amino acid sequence of SEQ ID NOs: 8 or 25, or SEQ ID NOs: 8 or 25 with 3, 2 or 1 amino acid substitution(s) thereto; or
ii) a LCDR2 amino acid sequence, as defined by Chothia or Kabat, from a V_{L} domain according to SEQ ID Nos: 6, 23 or 31, or SEQ ID Nos: 6, 23 or 31 with 3, 2 or 1 amino acid substitution(s) thereto.

8. The antibody or antigen-binding fragment thereof, according to claim 3, comprising:
a V_{H} region selected from SEQ ID NO: 2, 10, 13, 16, 19 or 27, or an amino acid sequence that is at least 80%, 85%, 90%, 92%, 93%, 95%, 97%, 98%, 99% or 100% identical thereto; and
a V_{L} region according to SEQ ID NO: 6, 23 or 31, or an amino acid sequence that is at least 80%, 85%, 90%, 92%, 93%, 95%, 97%, 98%, 99% or 100% identical thereto.

9.-17. (canceled)

18. The antibody or antigen-binding fragment thereof according to claim 3, wherein
i. the V_{H} region comprises an amino acid sequence which is identical or at least 90% identical to SEQ ID NO: 2 and the V_{L} region comprises an amino acid sequence which is identical or at least 90% identical to SEQ ID NO: 6;
ii. the V_{H} region comprises an amino acid sequence which is identical or at least 90% identical to SEQ ID NO: 10 and the V_{L} region comprises an amino acid sequence which is identical or at least 90% identical to SEQ ID NO: 6;
iii. the V_{H} region comprises an amino acid sequence which is identical or at least 90% identical SEQ ID NO: 13 and the V_{L} region comprises an amino acid sequence which is identical or at least 90% identical to SEQ ID NO: 6; or
iv. the V_{H} region comprises an amino acid sequence which is identical or at least 90% identical to SEQ ID NO: 16 and the V_{L} region comprises an amino acid sequence which is identical or at least 90% identical to SEQ ID NO: 6; or
v. the V_{H} region comprises an amino acid sequence which is identical or at least 90% identical to SEQ ID NO: 19 and the V_{L} region comprises an amino acid sequence which is identical or at least 90% identical to SEQ ID NO: 23; or
vi. the V_{H} region comprises an amino acid sequence which is identical or at least 90% identical to SEQ ID NO: 27 and the V_{L} region comprises an amino acid sequence which is identical or at least 90% identical to SEQ ID NO: 31.

19.-23. (canceled)

24. The antibody or antigen binding fragment thereof according to claim 8, comprising a heavy chain variable domain (V_{H}) and a light chain variable domain (V_{L}),
i. wherein the V_{H} comprises:
(a) the HCDR1 having the amino acid sequence of SEQ ID NO: 3;
(b) the HCDR2 having the amino acid sequence of SEQ ID NO: 4; and
(c) the HCDR3 having the amino acid sequence of SEQ ID NO: 5;
and wherein the V_{L} comprises:
(d) the LCDR1 having the amino acid sequence of SEQ ID NO: 7;
(e) the LCDR2 having the amino acid sequence of SEQ ID NO: 8; and
(f) the LCDR3 having the amino acid sequence of SEQ ID NO: 9; or
ii. wherein the V_{H} comprises:
(a) the HCDR1 having the amino acid sequence of SEQ ID NO: 11;
(b) the HCDR2 having the amino acid sequence of SEQ ID NO: 12;
and
(c) the HCDR3 having the amino acid sequence of SEQ ID NO: 5;
and wherein the V_{L} comprises:
(d) the LCDR1 having the amino acid sequence of SEQ ID NO: 7;
(e) the LCDR2 having the amino acid sequence of SEQ ID NO: 8; and
(f) the LCDR3 having the amino acid sequence of SEQ ID NO: 9; or
iii. wherein the V_{H} comprises:
(a) the HCDR1 having the amino acid sequence of SEQ ID NO: 14;
(b) the HCDR2 having the amino acid sequence of SEQ ID NO: 15; and
(c) the HCDR3 having the amino acid sequence of SEQ ID NO: 5;
and wherein the V_{L} comprises:
(d) the LCDR1 having the amino acid sequence of SEQ ID NO: 7;
(e) the LCDR2 having the amino acid sequence of SEQ ID NO: 8; and
(f) the LCDR3 having the amino acid sequence of SEQ ID NO: 9; or
iv. wherein the V_{H} comprises:
(a) the HCDR1 having the amino acid sequence of SEQ ID NO: 17;
(b) the HCDR2 having the amino acid sequence of SEQ ID NO: 18;
and
(c) the HCDR3 having the amino acid sequence of SEQ ID NO: 5;
and wherein the V_{L} comprises:
(d) the LCDR1 having the amino acid sequence of SEQ ID NO: 7;
(e) the LCDR2 having the amino acid sequence of SEQ ID NO: 8; and
(f) the LCDR3 having the amino acid sequence of SEQ ID NO: 9; or
v. wherein the V_{H} comprises:
(a) the HCDR1 having the amino acid sequence of SEQ ID NO: 20;
(b) the HCDR2 having the amino acid sequence of SEQ ID NO: 21;
and
(c) the HCDR3 having the amino acid sequence of SEQ ID NO: 22;
and wherein the V_{L} comprises:
(d) the LCDR1 having the amino acid sequence of SEQ ID NO: 24;
(e) the LCDR2 having the amino acid sequence of SEQ ID NO: 25; and
(f) the LCDR3 having the amino acid sequence of SEQ ID NO: 26; or
vi. wherein the V_{H} comprises:
(a) the HCDR1 having the amino acid sequence of SEQ ID NO: 28;
(b) the HCDR2 having the amino acid sequence of SEQ ID NO: 29;
and
(c) the HCDR3 having the amino acid sequence of SEQ ID NO: 30;
and wherein the V_{L} comprises:
(d) the LCDR1 having the amino acid sequence of SEQ ID NO: 32;
(e) the LCDR2 having the amino acid sequence of SEQ ID NO: 25; and
(f) the LCDR3 having the amino acid sequence of SEQ ID NO: 33.

25.-45. (canceled)

46. The antibody or antigen-binding fragment thereof, according to claim 3, which specifically binds to PAR2.

47. The antibody or antigen-binding fragment thereof, according to claim 46, wherein the antibody or fragment does not bind to one or more of PAR1, PAR3, or PAR4, wherein binding is determined using flow cytometry or ELISA.

48. (canceled)

49. The method of claim 53, wherein 3 mg/kg of the antibody or antibody-binding fragment thereof suppresses PAR2 stimulant induced response in leucocytes by >95% over a period of 30 days, wherein suppression is measured by determining stimulant induced gene signatures.

50. The method of claim 53, wherein 1 mg/kg of the antibody or antigen-binding fragment thereof suppresses PAR2 peptide induced response in leucocytes by >90% over a period of 30 days, wherein suppression is measured by determining stimulant induced gene signatures.

51. (canceled)

52. (canceled)

53. A method of treating a PAR2-mediated disease or condition in a patient in need thereof, comprising administering to the patient, a therapeutically effective amount of an antibody or antigen-binding fragment thereof, wherein the PAR2-mediated disease or condition comprises one or more of: atopic dermatitis, asthma, cancer (various including breast, melanoma, head and neck), pain (chronic, inflammatory, post-operative, neuropathic, fracture, gout, cancer, gastrointestinal associated with inflammatory bowel disease), rheumatoid arthritis and associated uveitis, scleroderma, systemic lupus erythematosus, osteoarthritis, polymyalgia rheumatica, ankylosing spondylitis, Reiter's disease, psoriatic arthritis, chronic Lyme arthritis, Still's disease, dermatomyositis, inclusion body myositis, polymyositis and lymphangioleiomyomatosis, and wherein antibody or antigen-binding fragment thereof comprising a V_{H} domain, the V_{H} domain comprising a HCDR3 selected from:
(a) a HCDR3 comprising or consisting of the amino acid sequence of SEQ ID NOs: 5, 22 or 30; or SEQ ID NO: 5, 22 or 30 with 3, 2 or 1 amino acid substitutions thereto;
(b) a HCDR3 comprising an amino acid sequence that is at least 80%, 85%, 90%, 92%, 93%, 95%, 97%, 98%, 99% or 100% identical to SEQ ID NOs: 5, 22 or 30; or
(c) a HCDR3 amino acid sequence as defined by Kabat or Chothia and is from a V_{H} domain selected from SEQ ID NOs: 2, 10, 13, 16, 19 or 27.

54. (canceled)

55. The method of claim 53, wherein the pain is independently selected from chronic pain, inflammatory pain, post-operative pain, neuropathic pain, fracture associated pain, gout associated pain, cancer associated pain, and gastrointestinal pain associated with inflammatory bowel disease etc.)

56. The method according to claim 5553, further comprising administering a further therapy, selected from the group consisting of: anti-inflammatory drugs, analgesics, nonsteroidal anti-inflammatory drugs (NSAIDs), corticosteroids, hyaluronic acids, acetaminophens, codeine, lorcet, lortab, vicodin, hydrocodone, morphine, oxycontin, roxicodone, percocet, aspirin, celecoxib, pregabalin, abatacept, adalimumab, anakinra, certolizumab, etanercept, golimumab, infliximab, rituximab, tocilizumab and tofacitinib.

57. A pharmaceutical composition comprising an antibody or fragment as defined in claim 3 and a pharmaceutically acceptable excipient, diluent or carrier.

58.-61. (canceled)

62. The pharmaceutical composition of claim 57, further comprising one or more further therapeutic agents independently selected from the group consisting of analgesics including anti-inflammatory drugs, paracetamol, opioids, amitriptyline, gabapentin; anticancer drugs including alkylating, antimetabolites, antibiotics and enzymes, natural agents, hormones and antagonists, hyroxyurea, immunomodulators, tyrosine kinase inhibitors, biological response modifiers, molecularly targeted therapies, platinum based therapies.


SEQUENCE LISTING (74 sequences)

SEQ ID NO: 1 - protein, 397 aa, Homo sapiens
MRSPSAAWLLGAAILLAASLSCSGTIQGTNRSSKGRSLIGKVDGTSHVTGKGVTVETVFSVDEFSASVLTGKLTTVFLPIVYTIVFVVGLPSNGMALWVFLFRTKKKHPAVIYMANLALADLLSVIWFPLKIAYHIHGNNWIYGEALCNVLIGFFYGNMYCSILFMTCLSVQRYWVIVNPMGHSRKKANIAIGISLAIWLLILLVTIPLYVVKQTIFIPALNITTCHDVLPEQLLVGDMFNYFLSLAIGVFLFPAFLTASAYVLMIRMLRSSAMDENSEKKRKRAIKLIVTVLAMYLICFTPSNLLLVVHYFLIKSQGQSHVYALYIVALCLSTLNSCIDPFVYYFVSHDFRDHAKNALLCRSVRTVKQMQVSLTSKKHSRKSSSYSSSSTTVKTSY

SEQ ID NO: 2 - protein, 126 aa, synthetic construct
  REGION 1..126: note = Derived from the Ylanthia phage antibody library.
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGFHYTHSGKRYYYPFDIWGQGTLVTVSS

SEQ ID NO: 3 - protein, 5 aa, synthetic construct
  REGION 1..5: note = Derived from the Ylanthia phage antibody library.
SYAMS

SEQ ID NO: 4 - protein, 17 aa, synthetic construct
  REGION 1..17: note = Derived from the Ylanthia phage antibody library.
AISGSGGSTYYADSVKG

SEQ ID NO: 5 - protein, 17 aa, synthetic construct
  REGION 1..17: note = Derived from the Ylanthia phage antibody library.
GFHYTHSGKRYYYPFDI

SEQ ID NO: 6 - protein, 113 aa, synthetic construct
  REGION 1..113: note = Derived from the Ylanthia phage antibody library.
QSALTQPASVSGSPGQSITISCTGTSSDVGSYNLVSWYQQHPGKAPKLMIYEGSKRPSGVSNRFSGSKSGNTASLTISGLQAEDEADYYCSQYATFIAFAVFGGGTKLTVLGQ

SEQ ID NO: 7 - protein, 14 aa, synthetic construct
  REGION 1..14: note = Derived from the Ylanthia phage antibody library.
TGTSSDVGSYNLVS

SEQ ID NO: 8 - protein, 7 aa, synthetic construct
  REGION 1..7: note = Derived from the Ylanthia phage antibody library.
EGSKRPS

SEQ ID NO: 9 - protein, 11 aa, synthetic construct
  REGION 1..11: note = Derived from the Ylanthia phage antibody library.
SQYATFIAFAV

SEQ ID NO: 10 - protein, 126 aa, synthetic construct
  REGION 1..126: note = Derived from the Ylanthia phage antibody library.
EVQLLESGGGLVQPGGSLRLSCAASGFSFSRYAITWVRQAPGKGLEWVSTISALGLETYYAESVRGRFTISRDNSKNTLYLQMNSLRAEDTAVYYCARGFHYTHSGKRYYYPFDIWGQGTLVTVSS

SEQ ID NO: 11 - protein, 5 aa, synthetic construct
  REGION 1..5: note = Derived from the Ylanthia phage antibody library.
RYAIT

SEQ ID NO: 12 - protein, 17 aa, synthetic construct
  REGION 1..17: note = Derived from the Ylanthia phage antibody library.
TISALGLETYYAESVRG

SEQ ID NO: 13 - protein, 126 aa, synthetic construct
  REGION 1..126: note = Derived from the Ylanthia phage antibody library.
EVQLLESGGGLVQPGGSLRLSCAASGFTFSRYAMTWVRQAPGKGLEWVSTISGLGQEAYYAGSVKGRFTISRDNSKNTLYLQMNSLRAEDTAVYYCARGFHYTHSGKRYYYPFDIWGQGTLVTVSS

SEQ ID NO: 14 - protein, 5 aa, synthetic construct
  REGION 1..5: note = Derived from the Ylanthia phage antibody library.
RYAMT

SEQ ID NO: 15 - protein, 17 aa, synthetic construct
  REGION 1..17: note = Derived from the Ylanthia phage antibody library.
TISGLGQEAYYAGSVKG

SEQ ID NO: 16 - protein, 126 aa, synthetic construct
  REGION 1..126: note = Derived from the Ylanthia phage antibody library.
EVQLLESGGGLVQPGGSLRLSCAASGFSFTRYAISWVRQAPGKGLEWVSVISGHGSEANYAESVKGRFTISRDNSKNTLYLQMNSLRAEDTAVYYCARGFHYTHSGKRYYYPFDIWGQGTLVTVSS

SEQ ID NO: 17 - protein, 5 aa, synthetic construct
  REGION 1..5: note = Derived from the Ylanthia phage antibody library.
RYAIS

SEQ ID NO: 18 - protein, 17 aa, synthetic construct
  REGION 1..17: note = Derived from the Ylanthia phage antibody library.
VISGHGSEANYAESVKG

SEQ ID NO: 19 - protein, 116 aa, synthetic construct
  REGION 1..116: note = Derived from the Ylanthia phage antibody library.
EVQLLESGGGLVQPGGSLRLSCAASGFTFSSYAISWVRQAPGKGLEWVSAISGSGGSTYYAESVKGRFTISRDNSKNTLYLQMNSLRAEDTAVYYCARGYAEFGVWGQGTLVTVSS

SEQ ID NO: 20 - protein, 5 aa, synthetic construct
  REGION 1..5: note = Derived from the Ylanthia phage antibody library.
SYAIS

SEQ ID NO: 21 - protein, 17 aa, synthetic construct
  REGION 1..17: note = Derived from the Ylanthia phage antibody library.
AISGSGGSTYYAESVKG

SEQ ID NO: 22 - protein, 7 aa, synthetic construct
  REGION 1..7: note = Derived from the Ylanthia phage antibody library.
GYAEFGV

SEQ ID NO: 23 - protein, 109 aa, synthetic construct
  REGION 1..109: note = Derived from the Ylanthia phage antibody library.
DIQMTQSPSSLSASVGDRVTITCRASQSISSYLNWYQQKPGKAPKLLIYAASSLQSGVPSRFSGSGSGTDFTLTISSLQPEDFATYYCQQTWGGPQTFGQGTKVEIKRT

SEQ ID NO: 24 - protein, 11 aa, synthetic construct
  REGION 1..11: note = Derived from the Ylanthia phage antibody library.
RASQSISSYLN

SEQ ID NO: 25 - protein, 7 aa, synthetic construct
  REGION 1..7: note = Derived from the Ylanthia phage antibody library.
AASSLQS

SEQ ID NO: 26 - protein, 9 aa, synthetic construct
  REGION 1..9: note = Derived from the Ylanthia phage antibody library.
QQTWGGPQT

SEQ ID NO: 27 - protein, 119 aa, synthetic construct
  REGION 1..119: note = Derived from the Ylanthia phage antibody library.
EVQLVESGGGLVKPGGSLRLSCAASGFTFSSYSINWVRQAPGKGLEWVSSISSSSSYTYYAESVKGRFTISRDNAKNSLYLQMNSLRAEDTAVYYCARAASGFAIFDYWGQGTLVTVSS

SEQ ID NO: 28 - protein, 5 aa, synthetic construct
  REGION 1..5: note = Derived from the Ylanthia phage antibody library.
SYSIN

SEQ ID NO: 29 - protein, 17 aa, synthetic construct
  REGION 1..17: note = Derived from the Ylanthia phage antibody library.
SISSSSSYTYYAESVKG

SEQ ID NO: 30 - protein, 10 aa, synthetic construct
  REGION 1..10: note = Derived from the Ylanthia phage antibody library.
AASGFAIFDY

SEQ ID NO: 31 - protein, 109 aa, synthetic construct
  REGION 1..109: note = Derived from the Ylanthia phage antibody library.
DIQMTQSPSSVSASVGDRVTITCRASQGISSWLAWYQQKPGKAPKLLIYAASSLQSGVPSRFSGSGSGTDFTLTISSLQPEDFATYYCQQTHYVPLTFGQGTKVEIKRT

SEQ ID NO: 32 - protein, 11 aa, synthetic construct
  REGION 1..11: note = Derived from the Ylanthia phage antibody library.
RASQGISSWLA

SEQ ID NO: 33 - protein, 9 aa, synthetic construct
  REGION 1..9: note = Derived from the Ylanthia phage antibody library.
QQTHYVPLT

SEQ ID NO: 34 - DNA, 651 bp, synthetic construct
  misc_feature 1..651: note = Derived from the Ylanthia phage antibody library.
  source 1..651: mol_type = other DNA
cagagcgccctgacccagccagccagcgttagcggtagcccaggccagagcattaccattagctgcaccggcaccagcagcgacgtgggcagctataacctggttagctggtatcagcagcatccgggcaaagccccgaaactgatgatctatgaaggcagcaaacgcccgagcggcgttagcaaccgctttagtggcagcaaaagcggcaacaccgccagcctgaccattagcggcctgcaagccgaagacgaagccgattattactgctcccagtacgctactttcatcgctttcgctgtgtttggcggcggtaccaagctgaccgtgctgggccagcccaaagccgcccctagcgtgaccctgttccccccaagcagcgaggaactccaggccaacaaggccaccctcgtgtgcctgatcagcgacttctaccctggcgccgtgaccgtggcctggaaggccgatagcagccctgtgaaggccggcgtggaaaccaccacccccagcaagcagagcaacaacaaatacgccgccagcagctacctgagcctgacccccgagcagtggaagtcccacagatcctacagctgccaggtcacacacgagggcagcaccgtggaaaagaccgtggcccccaccgagtgcagc

SEQ ID NO: 35 - DNA, 1368 bp, synthetic construct
  misc_feature 1..1368: note = Derived from the Ylanthia phage antibody library.
  source 1..1368: mol_type = other DNA
gaagtgcagctgctggaaagcggtggcggtctggtgcagccaggtggtagcctgcgcctgagctgtgccgcaagcggctttacctttagcagctatgccatgagctgggtgcgccaagcaccaggcaaaggcctggaatgggtgagcgccattagcggcagcggtggcagcacctattatgccgatagcgtgaaaggtcgctttaccattagtcgcgataacagcaaaaacaccctgtatctgcaaatgaacagcctgcgggcagaagataccgcagtttattattgcgcgcgaggattccactacactcactccggtaaacgttactactatccttttgacatttggggccagggcaccctggttactgtctcgagcgcgtcgaccaaaggccccagcgtgttccctctggcccccagcagcaagagcacctctggcggaacagccgccctgggctgcctggtcaaggactacttccccgagcccgtgaccgtgtcctggaactctggcgccctgaccagcggcgtgcacacctttccagccgtgctccagagcagcggcctgtacagcctgagcagcgtcgtgaccgtgcccagcagcagcctgggcacccagacctacatctgcaacgtgaaccacaagcccagcaacacaaaggtggacaagcgggtggaacccaagagctgcgacaagacccacacctgtcccccctgccctgcccctgaagcggagggagccccctccgtgttcctgttccccccaaagcctaaggacaccctgatgatcagccggacccccgaagtgacctgcgtggtggtggacgtgtcccacgaggaccctgaagtgaagtttaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgccttcctccatcgagaaaaccatcagcaaggccaaaggccagccccgcgagccccaggtgtacacactgccccctagccgggaagagatgaccaagaaccaggtgtccctgacctgcctcgtgaagggcttctaccccagcgacattgccgtggaatgggagagcaacggccagcccgagaacaactacaagaccaccccccctgtgctggacagcgacggctcattcttcctgtacagcaagctgaccgtggacaagagccggtggcagcagggcaacgtgttcagctgctccgtgatgcacgaggccctgcacaaccactacacccagaagtccctgagcctgagccccggcaag

SEQ ID NO: 36 - DNA, 1368 bp, synthetic construct
  misc_feature 1..1368: note = Derived from the Ylanthia phage antibody library.
  source 1..1368: mol_type = other DNA
gaagtgcagctgctggaaagcggtggcggtctggtgcagccaggtggtagcctgcgcctgagctgtgccgcaagcgggttttctttctccagatacgcaatcacctgggtgcgccaagcaccaggcaaaggcctggaatgggtgagcaccatttccgcactgggtctggagacatattacgcggagtccgtccgcggccgctttaccattagtcgcgataacagcaaaaacaccctgtatctgcaaatgaacagcctgcgggcagaagataccgcagtttattattgcgcgcgaggattccactacactcactccggtaaacgttactactatccttttgacatttggggccagggcaccctggttactgtctcgagcgcgtcgaccaaaggccccagcgtgttccctctggcccccagcagcaagagcacctctggcggaacagccgccctgggctgcctggtcaaggactacttccccgagcccgtgaccgtgtcctggaactctggcgccctgaccagcggcgtgcacacctttccagccgtgctccagagcagcggcctgtacagcctgagcagcgtcgtgaccgtgcccagcagcagcctgggcacccagacctacatctgcaacgtgaaccacaagcccagcaacacaaaggtggacaagcgggtggaacccaagagctgcgacaagacccacacctgtcccccctgccctgcccctgaagcggagggagccccctccgtgttcctgttccccccaaagcctaaggacaccctgatgatcagccggacccccgaagtgacctgcgtggtggtggacgtgtcccacgaggaccctgaagtgaagtttaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgccttcctccatcgagaaaaccatcagcaaggccaaaggccagccccgcgagccccaggtgtacacactgccccctagccgggaagagatgaccaagaaccaggtgtccctgacctgcctcgtgaagggcttctaccccagcgacattgccgtggaatgggagagcaacggccagcccgagaacaactacaagaccaccccccctgtgctggacagcgacggctcattcttcctgtacagcaagctgaccgtggacaagagccggtggcagcagggcaacgtgttcagctgctccgtgatgcacgaggccctgcacaaccactacacccagaagtccctgagcctgagccccggcaag

SEQ ID NO: 37 - DNA, 1368 bp, synthetic construct
  misc_feature 1..1368: note = Derived from the Ylanthia phage antibody library.
  source 1..1368: mol_type = other DNA
gaagtgcagctgctggaaagcggtggcggtctggtgcagccaggtggtagcctgcgcctgagctgtgccgcaagcggctttaccttcagcagatatgcaatgacttgggtgcgccaagcaccaggcaaaggcctggaatgggtgagtaccataagcgggctgggacaagaggcttactacgcaggctccgtcaaaggccgctttaccattagtcgcgataacagcaaaaacaccctgtatctgcaaatgaacagcctgcgggcagaagataccgcagtttattattgcgcgcgaggattccactacactcactccggtaaacgttactactatccttttgacatttggggccagggcaccctggttactgtctcgagcgcgtcgaccaaaggccccagcgtgttccctctggcccccagcagcaagagcacctctggcggaacagccgccctgggctgcctggtcaaggactacttccccgagcccgtgaccgtgtcctggaactctggcgccctgaccagcggcgtgcacacctttccagccgtgctccagagcagcggcctgtacagcctgagcagcgtcgtgaccgtgcccagcagcagcctgggcacccagacctacatctgcaacgtgaaccacaagcccagcaacacaaaggtggacaagcgggtggaacccaagagctgcgacaagacccacacctgtcccccctgccctgcccctgaagcggagggagccccctccgtgttcctgttccccccaaagcctaaggacaccctgatgatcagccggacccccgaagtgacctgcgtggtggtggacgtgtcccacgaggaccctgaagtgaagtttaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgccttcctccatcgagaaaaccatcagcaaggccaaaggccagccccgcgagccccaggtgtacacactgccccctagccgggaagagatgaccaagaaccaggtgtccctgacctgcctcgtgaagggcttctaccccagcgacattgccgtggaatgggagagcaacggccagcccgagaacaactacaagaccaccccccctgtgctggacagcgacggctcattcttcctgtacagcaagctgaccgtggacaagagccggtggcagcagggcaacgtgttcagctgctccgtgatgcacgaggccctgcacaaccactacacccagaagtccctgagcctgagccccggcaag

SEQ ID NO: 38 - DNA, 1368 bp, synthetic construct
  misc_feature 1..1368: note = Derived from the Ylanthia phage antibody library.
  source 1..1368: mol_type = other DNA
gaagtgcagctgctggaaagcggtggcggtctggtgcagccaggtggtagcctgcgcctgagctgtgccgcaagcgggttctccttcaccagatatgcaatttcctgggtgcgccaagcaccaggcaaaggcctggaatgggtgagtgtgataagcgggcatggttccgaggctaactacgccgagagcgtcaagggtcgctttaccattagtcgcgataacagcaaaaacaccctgtatctgcaaatgaacagcctgcgggcagaagataccgcagtttattattgcgcgcgaggattccactacactcactccggtaaacgttactactatccttttgacatttggggccagggcaccctggttactgtctcgagcgcgtcgaccaaaggccccagcgtgttccctctggcccccagcagcaagagcacctctggcggaacagccgccctgggctgcctggtcaaggactacttccccgagcccgtgaccgtgtcctggaactctggcgccctgaccagcggcgtgcacacctttccagccgtgctccagagcagcggcctgtacagcctgagcagcgtcgtgaccgtgcccagcagcagcctgggcacccagacctacatctgcaacgtgaaccacaagcccagcaacacaaaggtggacaagcgggtggaacccaagagctgcgacaagacccacacctgtcccccctgccctgcccctgaagcggagggagccccctccgtgttcctgttccccccaaagcctaaggacaccctgatgatcagccggacccccgaagtgacctgcgtggtggtggacgtgtcccacgaggaccctgaagtgaagtttaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgccttcctccatcgagaaaaccatcagcaaggccaaaggccagccccgcgagccccaggtgtacacactgccccctagccgggaagagatgaccaagaaccaggtgtccctgacctgcctcgtgaagggcttctaccccagcgacattgccgtggaatgggagagcaacggccagcccgagaacaactacaagaccaccccccctgtgctggacagcgacggctcattcttcctgtacagcaagctgaccgtggacaagagccggtggcagcagggcaacgtgttcagctgctccgtgatgcacgaggccctgcacaaccactacacccagaagtccctgagcctgagccccggcaag

SEQ ID NO: 39 - protein, 6 aa, synthetic construct
  REGION 1..6: note = Artificial ligand which mimics the exposed tethered ligand.
SLIGKV

SEQ ID NO: 40 - protein, 6 aa, synthetic construct
  REGION 1..6: note = Artificial ligand which mimics the exposed tethered ligand.
SLIGRL

SEQ ID NO: 41 - protein, 5 aa, Homo sapiens
VETVF

SEQ ID NO: 42 - protein, 18 aa, Homo sapiens
SVDEFSASVLTGKLTTVF

SEQ ID NO: 43 - protein, 6 aa, Homo sapiens
LLVVHY

SEQ ID NO: 44 - protein, 15 aa, Homo sapiens
FLIKSQGQSHVYALY

SEQ ID NO: 45 - protein, 24 aa, Homo sapiens
VTVETVFSVDEFSASVLTGKLTTV

SEQ ID NO: 46 - protein, 7 aa, synthetic construct
  REGION 1..7: note = Derived from the Ylanthia phage antibody library.
GFTFSSY

SEQ ID NO: 47 - protein, 6 aa, synthetic construct
  REGION 1..6: note = Derived from the Ylanthia phage antibody library.
SGSGGS

SEQ ID NO: 48 - protein, 17 aa, synthetic construct
  REGION 1..17: note = Derived from the Ylanthia phage antibody library.
GFHYTHSGKRYYYPFDI

SEQ ID NO: 49 - protein, 10 aa, synthetic construct
  REGION 1..10: note = Derived from the Ylanthia phage antibody library.
TSSDVGSYNL

SEQ ID NO: 50 - protein, 8 aa, synthetic construct
  REGION 1..8: note = Derived from the Ylanthia phage antibody library.
YATFIAFA

SEQ ID NO: 51 - protein, 7 aa, synthetic construct
  REGION 1..7: note = Derived from the Ylanthia phage antibody library.
GFSFSRY

SEQ ID NO: 52 - protein, 6 aa, synthetic construct
  REGION 1..6: note = Derived from the Ylanthia phage antibody library.
SALGLE

SEQ ID NO: 53 - protein, 7 aa, synthetic construct
  REGION 1..7: note = Derived from the Ylanthia phage antibody library.
GFTFSRY

SEQ ID NO: 54 - protein, 6 aa, synthetic construct
  REGION 1..6: note = Derived from the Ylanthia phage antibody library.
SGLGQE

SEQ ID NO: 55 - protein, 7 aa, synthetic construct
  REGION 1..7: note = Derived from the Ylanthia phage antibody library.
GFSFTRY

SEQ ID NO: 56 - protein, 6 aa, synthetic construct
  REGION 1..6: note = Derived from the Ylanthia phage antibody library.
SGHGSE

SEQ ID NO: 57 - protein, 7 aa, synthetic construct
  REGION 1..7: note = Derived from the Ylanthia phage antibody library.
GFTFSSY

SEQ ID NO: 58 - protein, 6 aa, synthetic construct
  REGION 1..6: note = Derived from the Ylanthia phage antibody library.
SGSGGS

SEQ ID NO: 59 - protein, 7 aa, synthetic construct
  REGION 1..7: note = Derived from the Ylanthia phage antibody library.
GYAEFGV

SEQ ID NO: 60 - protein, 7 aa, synthetic construct
  REGION 1..7: note = Derived from the Ylanthia phage antibody library.
SQSISSY

SEQ ID NO: 61 - protein, 5 aa, synthetic construct
  REGION 1..5: note = Derived from the Ylanthia phage antibody library.
WGGPQ

SEQ ID NO: 62 - protein, 7 aa, synthetic construct
  REGION 1..7: note = Derived from the Ylanthia phage antibody library.
GFTFSSY

SEQ ID NO: 63 - protein, 6 aa, synthetic construct
  REGION 1..6: note = Derived from the Ylanthia phage antibody library.
SSSSSY

SEQ ID NO: 64 - protein, 10 aa, synthetic construct
  REGION 1..10: note = Derived from the Ylanthia phage antibody library.
AASGFAIFDY

SEQ ID NO: 65 - protein, 7 aa, synthetic construct
  REGION 1..7: note = Derived from the Ylanthia phage antibody library.
SQGISSW

SEQ ID NO: 66 - protein, 5 aa, synthetic construct
  REGION 1..5: note = Derived from the Ylanthia phage antibody library.
HYVPL

SEQ ID NO: 67 - protein, 425 aa, Homo sapiens
MGPRRLLLVAACFSLCGPLLSARTRARRPESKATNATLDPRSFLLRNPNDKYEPFWEDEEKNESGLTEYRLVSINKSSPLQKQLPAFISEDASGYLTSSWLTLFVPSVYTGVFVVSLPLNIMAIVVFILKMKVKKPAVVYMLHLATADVLFVSVLPFKISYYFSGSDWQFGSELCRFVTAAFYCNMYASILLMTVISIDRFLAVVYPMQSLSWRTLGRASFTCLAIWALAIAGVVPLLLKEQTIQVPGLNITTCHDVLNETLLEGYYAYYFSAFSAVFFFVPLIISTVCYVSIIRCLSSSAVANRSKKSRALFLSAAVFCIFIICFGPTNVLLIAHYSFLSHTSTTEAAYFAYLLCVCVSSISCCIDPLIYYYASSECQRYVYSILCCKESSDPSSYNSSGQLMASKMDTCSSNLNNSIYKKLLT

SEQ ID NO: 68 - protein, 374 aa, Homo sapiens
MKALIFAAAGLLLLLPTFCQSGMENDTNNLAKPTLPIKTFRGAPPNSFEEFPFSALEGWTGATITVKIKCPEESASHLHVKNATMGYLTSSLSTKLIPAIYLLVFVVGVPANAVTLWMLFFRTRSICTTVFYTNLAIADFLFCVTLPFKIAYHLNGNNWVFGEVLCRATTVIFYGNMYCSILLLACISINRYLAIVHPFTYRGLPKHTYALVTCGLVWATVFLYMLPFFILKQEYYLVQPDITTCHDVHNTCESSSPFQLYYFISLAFFGFLIPFVLIIYCYAAIIRTLNAYDHRWLWYVKASLLILVIFTICFAPSNIILIIHHANYYYNNTDGLYFIYLIALCLGSLNSCLDPFLYFLMSKTRNHSTAYLTK

SEQ ID NO: 69 - protein, 385 aa, Homo sapiens
MWGRLLLWPLVLGFSLSGGTQTPSVYDESGSTGGGDDSTPSILPAPRGYPGQVCANDSDTLELPDSSRALLLGWVPTRLVPALYGLVLVVGLPANGLALWVLATQAPRLPSTMLLMNLAAADLLLALALPPRIAYHLRGQRWPFGEAACRLATAALYGHMYGSVLLLAAVSLDRYLALVHPLRARALRGRRLALGLCMAAWLMAAALALPLTLQRQTFRLARSDRVLCHDALPLDAQASHWQPAFTCLALLGCFLPLLAMLLCYGATLHTLAASGRRYGHALRLTAVVLASAVAFFVPSNLLLLLHYSDPSPSAWGNLYGAYVPSLALSTLNSCVDPFIYYYVSAEFRDKVRAGLFQRSPGDTVASKASAEGGSRGMGTHSSLLQ

SEQ ID NO: 70 - protein, 217 aa, synthetic construct
QSALTQPASVSGSPGQSITISCTGTSSDVGSYNLVSWYQQHPGKAPKLMIYEGSKRPSGVSNRFSGSKSGNTASLTISGLQAEDEADYYCSQYATFIAFAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 71 - protein, 456 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGFHYTHSGKRYYYPFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 456 aa, synthetic construct
EVQLLESGGGLVQPGGSLRLSCAASGFSFSRYAITWVRQAPGKGLEWVSTISALGLETYYAESVRGRFTISRDNSKNTLYLQMNSLRAEDTAVYYCARGFHYTHSGKRYYYPFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 456 aa, synthetic construct
EVQLLESGGGLVQPGGSLRLSCAASGFTFSRYAMTWVRQAPGKGLEWVSTISGLGQEAYYAGSVKGRFTISRDNSKNTLYLQMNSLRAEDTAVYYCARGFHYTHSGKRYYYPFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 456 aa, synthetic construct
EVQLLESGGGLVQPGGSLRLSCAASGFSFTRYAISWVRQAPGKGLEWVSVISGHGSEANYAESVKGRFTISRDNSKNTLYLQMNSLRAEDTAVYYCARGFHYTHSGKRYYYPFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK